(19) United States
(12) Patent Application Publication
(10) Pub. No.: US 2004/0266871 A1
(43) Pub. Date: Dec. 30, 2004

(54) Methods for treating alzheimer's disease using quinaldoyl-amine derivatives of oxo-and hydroxy-substituted hydrocarbons

(76) Inventor: Heinrich Josef Schostarez, Portage, MI (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/488,019

(22) PCT Filed: Aug. 28, 2002
(86) PCT No.: PCT/US02/27408

Related U.S. Application Data
(60) Provisional application No. 60/315,550, filed on Aug. 28, 2001.

(51) Int. Cl. A61K 31/205; A61K 31/165

(52) U.S. Cl. 514/554; 514/620

(57) ABSTRACT

Disclosed are methods for treating Alzheimer's disease, and other diseases, and/or inhibiting beta-secretase enzyme, inhibiting beta-secretase enzyme, and/or inhibiting deposition of A beta peptide in a mammal, by use of compounds of of compounds of formula (I) wherein R₁, R₂, R₃. and N, are defined herein.

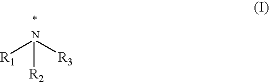

This application claims priority to U.S. Provisional Patent Application No. 60/315,550, filed on Aug. 28, 2001.

### FIELD OF THE INVENTION

The present invention relates to the treatment of Alzheimer's disease and other similar diseases, and more specifically to the use of compounds that inhibit beta-secretase, an enzyme that cleaves amyloid precursor protein to produce A beta peptide, a major component of the amyloid plaques found in the brains of Alzheimer's sufferers, in such methods.

### BACKGROUND OF THE INVENTION

Alzheimer's disease (AD) is a progressive degenerative disease of the brain primarily associated with aging. Clinical presentation of AD is characterized by loss of memory, cognition, reasoning, judgment, and orientation. As the disease progresses, motor, sensory, and linguistic abilities are also affected until there is global impairment of multiple cognitive functions. These cognitive losses occur gradually, but typically lead to severe impairment and eventual death in the range of four to twelve years.

Alzheimer's disease is characterized by two major pathologic observations in the brain: neurofibrillary tangles and beta amyloid (or neuritic) plaques, comprised predominantly of an aggregate of a peptide fragment know as A beta. Individuals with AD exhibit characteristic beta-amyloid deposits in the brain (beta amyloid plaques) and in cerebral blood vessels (beta amyloid angiopathy) as well as neurofibrillary tangles. Neurofibrillary tangles occur not only in Alzheimer's disease but also in other dementia-inducing disorders. On autopsy, large numbers of these lesions are generally found in areas of the human brain important for memory and cognition.

Smaller numbers of these lesions in a more restricted anatomical distribution are found in the brains of most aged humans who do not have clinical AD. Amyloidogenic plaques and vascular amyloid angiopathy also characterize the brains of individuals with Trisomy 21 (Down's Syndrome), Hereditary Cerebral Hemorrhage with Amyloidosis of the Dutch-Type (HCHWA-D), and other neurodegenerative disorders. Beta-amyloid is a defining feature of AD, now believed to be a causative precursor or factor in the development of disease. Deposition of A beta in areas of the brain responsible for cognitive activities is a major factor in the development of AD. Beta-amyloid plaques are predominantly composed of amyloid beta peptide (A beta, also sometimes designated betaA4). A beta peptide is derived by proteolysis of the amyloid precursor protein (APP) and is comprised of 39-42 amino acids. Several proteases called secretases are involved in the processing of APP.

Cleavage of APP at the N-terminus of the A beta peptide by beta-secretase and at the C-terminus by one or more gamma-secretase constitutes the beta-amyloidogenic pathway, i.e. the pathway by which A beta is formed. Cleavage of APP by alpha-secretase produces alpha-sAPP, a secreted form of APP that does not result in beta-amyloid plaque formation. This alternate pathway precludes the formation of A beta peptide. A description of the proteolytic processing fragments of APP is found, for example, in U.S. Pat. Nos. 5,441,870; 5,721,130; and 5,942,400.

An aspartyl protease has been identified as the enzyme responsible for processing of APP at the beta-secretase cleavage site. The beta-secretase enzyme has been disclosed using varied nomenclature, including BACE, Asp, and Memapsin. See, for example, Sindha et al., 1999, Nature 402:537-554 (p501) and published PCT application WO00/17369.

Several lines of evidence indicate that progressive cerebral deposition of beta-amyloid peptide (A beta) plays a seminal role in the pathogenesis of AD and can precede cognitive symptoms by years or decades. See, for example, Selkoe, 1991, Neuron 6:487. Release of A beta from neuronal cells grown in culture and the presence of A beta in cerebrospinal fluid (CSF) of both normal individuals and AD subjects has been demonstrated. See, for example, Seubert et al., 1992, Nature 359:325-327.

It has been proposed that A beta peptide accumulates as a result of APP processing by beta-secretase, thus inhibition of this enzyme's activity is desirable for the treatment of AD. In vivo processing of APP at the beta-secretase cleavage site is thought to be a rate-limiting step in A beta production, and is thus a therapeutic target for the treatment of AD. See For example, Sabbagh, M., et al., 1997, Alz. Dis. Rev. 3, 1-19.

BACE1 knockout mice fail to produce A beta, and present a normal phenotype. When crossed with transgenic mice that over express APP, the progeny show reduced amounts of A beta in brain extracts as compared with control animals (Luo et al., 2001 Nature Neuroscience 4:231-232). This evidence further supports the proposal that inhibition of beta-secretase activity and reduction of A beta in the brain provides a therapeutic method for the treatment of AD and other beta amyloid disorders.

At present there are no effective treatments for halting, preventing, or reversing the progression of Alzheimer's disease. Therefore, there is an urgent need for pharmaceutical agents capable of slowing the progression of Alzheimer's disease and/or preventing it in the first place.

Compounds that are effective inhibitors of beta-secretase, that inhibit beta-secretase-mediated cleavage of APP, that are effective inhibitors of A beta production, and/or are effective to reduce amyloid beta deposits or plaques, are needed for the treatment and prevention of disease characterized by amyloid beta deposits or plaques, such as AD.

U.S. Pat. No. 5,679,688 discloses quinaldoyl-amine derivatives of oxo- and hydroxy-substituted hydrocarbons and suggests that such compounds can be used as HIV protease inhibitors for the treatment of AIDS. The disclosure of U.S. Pat. No. 5,679,688 is incorporated herein by reference in its entirety.

### SUMMARY OF INVENTION

The present invention relates to methods of treating a subject who has, or in preventing a subject from developing, a disease or condition selected from the group consisting of Alzheimer's disease, for helping prevent or delay the onset of Alzheimer's disease, for helping to slow the progression of Alzheimer's disease, for treating subjects with mild cognitive impairment (MCI) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, frontotemporal dementias with parkinsonism (FTDP), dementia associated with progressive supranuclear palsy, dementia associated with cortical basal degeneration, or diffuse Lewy body type of Alzheimer's disease and who is in need of such treatment which comprises administration of a therapeutically effective amount of a compound of formula (I):

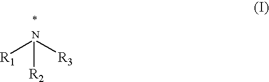

or pharmaceutically acceptable salts thereof, wherein: R₁ is a group R, wherein R is selected from the group consisting of hydrogen, —R′H, —R′C(O)OR″, —R′C(O)NH₂, —R′C(O)NHR″, —R′C(C)NR″R′″, —R′NHC (O)R″, —R′NR′″C(O)R″ or —R′C(O)R″, where R″ and R′″ are independently optionally substituted (C₁-C₁₈)alkyl, typically (C₁-C₁₂)alkyl; (C₃-C₁₈)cycloalkyl, typically (C₃-C₁₂)cycloalkyl; (C₃-C₁₈)cycloalkyl(C₁-C₁₈)alkyl, typically (C₃-C₁₂)cycloalkyl(C₁-C₆)alkyl; (C₆-C₂₄)aryl, typically (C₆-C₁₆)aryl; (C₇-C₂₅)aralkyl, typically (C₇-C₁₆)aralkyl; (C₂-C₁₈)alkenyl, typically (C₂-C₁₂)alkenyl; (C₈-C₂₆)aralkenyl, typically (C₈-C₁₆)aralkenyl; (C₂-C₁₈)alkynyl, typically (C₂-C₁₂)alkynyl; (C₈-C₂₆)aralkynyl, typically (C₈-C₁₆)aralkynyl; or heterocyclic, and where R′ is an optionally substituted divalent radical derived from (C₁-C₁₈)alkyl, typically (C₁-C₁₂)alkyl; (C₃-C₁₈)cycloalkyl, typically (C₃-C₁₂)cycloalkyl; (C₃-C₁₈)cycloalkyl (C₁-C₁₈)alkyl, typically (C₃-C₁₂)cycloalkyl(C₁-C₆)alkyl; (C₆-C₂₄)aryl, typically (C₆-C₁₆)aryl; (C₇-C₂₅)aralkyl, typically (C₇-C₁₆)aralkyl; (C₂-C₁₈)alkenyl, typically (C₂-C₁₂)alkenyl; (C₈-C₂₆)aralkenyl, typically (C₈-C₁₆)aralkenyl; (C₂-C₁₈)alkynyl, typically (C₂-C₁₂)alkynyl; (C₈-C₂₆)aralkynyl, typically (C₈-C₁₆)aralkynyl; or heterocyclic,

or R₁ is

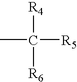

where R₄, R₅ and R₆ are independently a group R as defined above, or R₄ has the meaning of R as defined above and R₅ and R₆ taken together are ═O, ═S, ═NH or ═NR;

and R₂ is

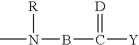

where R is as previously defined; D is O or S; Y is. hydrogen, —R or —OR, where R is as previously defined, or is an amino acid, aza-amino acid or peptide residue in which any functional group present is optionally protected; and B is optionally absent or is (C₁-C₆)alkylidene, wherein any one or more —CH₂— groups may be replaced by —NR—, —NH—, —O— or —S— provided that the compound of Formula (I) does not contain a chain of three or more atoms which are not carbon, and wherein any H atom may be substituted by a group R as previously defined; and optionally N*, N, R₁ and R taken together form a cyclic diazaalkane of the formula:

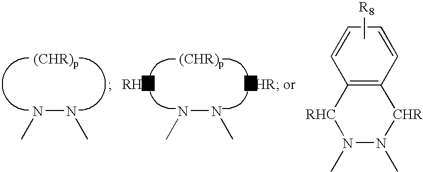

where p is 1 to 3, each R is independently as defined above and R₈ is R, —NH₂, —NHR, —NR₂, —COOH, —COOL, —CHO, —C(O)R, —CN, halo, —CF₃, −OL, —SR, —S(O)R, —S(O)₂R, —CONH₂, —CONHR, —CONR₂, NHOH, —NHOL, —NO₂, ═O, ═S or —NHNH₂, wherein each R is independently as defined above and each L is independently R or a hydroxyl protecting group which is labile in vivo; or R₂, N* and R₄ together form a saturated or unsaturated cyclic, bicyclic or fused ring system as defined hereinafter which may be additionally substituted by —C(O)Y, where Y is as previously defined

and R₃ is X—W-A′-Q-A-, wherein: A′ and A independently are absent or (C₁-C₈)alkylidene, typically (C₁-C₄)alkylidene which may be substituted with one or more substituents R as previously defined;

Q is

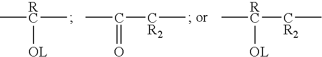

where L and each R, independently of the others, are as previously defined,

and optionally Q and A together, or Q and A′ together, or A′, Q and A together form part of a saturated or unsaturated cyclic, bicyclic or fused ring system as defined hereinafter;

W is absent or is N(R), O or S, wherein R is as previously defined; and X is hydrogen, or X₁, where X₁ is Ra— or RbC(O)— or RbS(O)_{z}—, where z is 1 or 2 and Ra and Rb are independently (C₁-C₁₈)alkyl, typically (C₁-C₁₂)alkyl; (C₃-C₁₈)cycloalkyl, typically (C₃-C₁₂)cycloalkyl; (C₃-C₁₈)cycloalkyl(C₁-C₁₈)alkyl, typically (C₃-C₁₂)cycloalkyl(C₁-C₆)alkyl; heterocyclic; (C₁-C₁₈)alkylheterocyclic, typically (C₁-C₁₂)alkylheterocyclic; heterocyclic(C₆-C₂₄)aryloxy, typically heterocyclic(C₆-C₁₆)aryloxy; (C₁-C₁₈)alkoxy, typically (C₁-C₁₂)alkoxy; (C₁-C₁₈)alkoxy(C₁-C₁₈)alkyl, typically (C₁-C₁₂)alkoxy; (C₁-C₁₂)alkyl; (C₆-C₂₄)aryloxy (C₁-C₁₈)alkyl, typically (C₆-C₁₆)aryloxy (C₁-C₁₂)alkyl; (C₆-C₂₄)aryloxy(C₁-C₁₈)alkoxy, typically (C₆-C₁₆)aryloxy (C₁-C₁₂)alkoxy; (C₆-C₂₄)aryl, typically (C₆-C₁₆)aryl; (C₆-C₂₄)aryl(C₁-C₁₈)alkyl, typically (C₆-C₁₆)aryl(C₁-C₁₂)alkyl; (C₆-C₂₄)aryl (C₁-C₁₈)alkylheterocyclic, typically (C₆-C₁₆)aryl; (C₁-C₁₂)alkylheterocyclic; heterocyclicoxy(C₁-C₁₈)alkyl, typically heterocyclicoxy(C₁-C₁₂)alkyl; (C₁-C₁₈)alkylamino, typically (C₁-C₁₂)alkylamino; di(C₁-C₁₈)alkylamino, typically di(C₁-C₁₂)alkylamino; (C₆-C₂₄)arylamino, typically (C₆-C₁₆)arylamino; di(C₆-C₂₄)arylamino, typically di(C₆-C₁₆)arylamino; (C₇-C₂₅)aralkylamino, typically (C₇-C₁₂)aralkylamino or di(C₇-C₂₅)aralkylamino, typically di(C₇-C₁₂)aralkylamino; any of which may be optionally substituted as hereinbelow defined-or substituted with a group Re, where Re is a group of the formula:

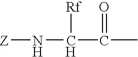

where Z has the meaning of Ra or Rb or is an acylated amino acid, azaamino acid or peptide residue, and Rf is the side-chain of a natural amino acid in Which any functional group present is optionally protected;

or X is Re as previously defined,

or X is an optionally protected amino acid, azaamino acid or peptide residue; or

when W is N(R), then X, N and the substituent R on N together may form a saturated or unsaturated cyclic, bicyclic or fused ring system as defined hereinbelow or N, A′ and the substituent R on N together form a saturated or unsaturated cyclic, bicyclic or fused ring system as defined hereinbelow.

Compounds employed with the methods of the invention can comprise two R substituents, not necessarily vicinal, taken together are optionally substituted (C₂-C₁₈)alkylidene, typically (C₂-C₈)alkylidene.

Compounds also employed with the methods of the invention can comprise compounds wherein the Z-NH bond shown is replaced by a modified isosteric bond, such as CH₃—NRa—, RaCH₂—NRa—, CH₃—CHRa—, HCH═CRa—, RaCH═CRa—, HCOCHRa—, RaCOCHRa—, HCHOHCHRa—, RaCHOHCHRa—, HNRaCO—, HCF═CRa—, RaCF═CRa—, RaS(O)—, RaS(O)₂—, RaP(O)ORa—, RaP(O)(ORa)CH₂—, RaP(O)(ORa)O—, RaP(O)(ORa)S—, wherein each Ra is independently as previously defined.

### DETAILED DESCRIPTION OF THE INVENTION

In one aspect, the present invention relates to methods of treating a subject who has, or in preventing a subject from developing, a disease or condition selected from the group consisting of Alzheimer's disease, for helping prevent or delay the onset of Alzheimer's disease, for helping to slow the progression of Alzheimer's disease, for treating subjects with mild cognitive impairment (MCI) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, frontotemporal dementias with parkinsonism (FTDP), dementia associated with progressive supranuclear palsy, dementia associated with cortical basal degeneration, or diffuse Lewy body type of Alzheimer's disease and who is in need of such treatment which comprises administration of a therapeutically effective amount of a compound of formula (I)

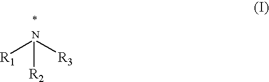

or pharmaceutically acceptable salts thereof, wherein: R₁ is a group R, wherein R is selected from the group consisting of hydrogen, —R′H, —R′C(O)OR″, —R′C(O)NH₂, —R′C(O)NHR″, —R′C(O)NR″R′″, —R′NHC(O)R″, —R′NR′″C(O)R″ or —R′C(O)R″, where R″ and R′″ are independently optionally substituted (C₁-C₁₈)alkyl, typically (C₁-C₁₂)alkyl; (C₃-C₁₈)cycloalkyl, typically (C₃-C₁₂)cycloalkyl; (C₃-C₁₈)cycloalkyl (C₁-C₁₈)alkyl, typically (C₃-C₁₂)cycloalkyl (C₁-C₆)alkyl; (C₁-C₂₄)aryl, typically (C₆-C₆)aryl; (C₇-C₂₅)aralkyl, typically (C₇-C₁₆)aralkyl; (C₂-C₁₈)alkenyl, typically (C₂-C₁₂)alkenyl; (C₈-C₂₆)aralkenyl, typically (C₈-C₁₆)aralkenyl; (C₂-C₁₈)alkynyl, typically (C₂-C₁₂)alkynyl; (C₃-C₂₆)aralkynyl, typically (C₈-C₁₆)aralkynyl; or heterocyclic, and where R′ is an optionally substituted divalent radical derived from (C₁-C₁₈)alkyl, typically (C₁-C₁₂)alkyl; (C₃-C₁₈)cycloalkyl, typically (C₁-C₁₂)cycloalkyl; (C₃-C₁₈)cycloalkyl (C₁-C₁₈)alkyl, typically (C₃-C₁₂)cycloalkyl (C₁-C₆)alkyl; (C₆-C₂₄)aryl, typically (C₆-C₁₆)aryl; (C₇-C₂₅)aralkyl, typically (C₇-C₁₆)aralkyl; (C₂-C₁₈)alkenyl, typically (C₂-C₁₂)alkenyl; (C₈-C₂₆)aralkenyl, typically (C₈-C₁₆)aralkenyl; (C₂-C₁₈)alkynyl, typically (C₂-C₁₂)alkynyl; (C₈-C₂₆)aralkynyl, typically (C₈-C₁₆)aralkynyl; or heterocyclic,

or R₁ is

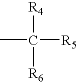

where R₄, R₅ and R₆ are independently a group R as defined above, or R4 has the meaning of R as defined above and R₅ and R₆ taken together are ═O, ═S, ═NH or ═NR;

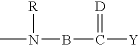

where R is as previously defined; D is O or S; Y is hydrogen, —R or —OR, where R is as previously defined, or is an amino acid, aza-amino acid or peptide residue in which any functional group present is optionally protected; and B is optionally absent or is (C₁-C₆)alkylidene, wherein any one or more —CH₂— groups may be replaced by —NR—, —NH—, —O— or —S— provided that the compound of Formula (I) does not contain a chain of three or more atoms which are not carbon, and wherein any H atom may be substituted by a group R as previously defined; and optionally N*, N, R₁ and R taken together form a cyclic diazaalkane of the formula:

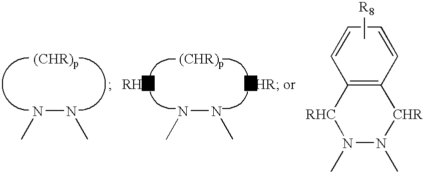

where p is 1 to 3, each R is independently as defined above and R₈ is R, —NH₂, —NHR, —NR₂, —COOH, —COOL, —CHO, —C(O)R, —CN, halo, —CF₃, —OL, —SR, —S(O)R, —S(O)₂R, —CONH₂, —CONHR, -CONR₂, —NHOH, —NHOL, —NO₂, ═O, ═S or —NHNH₂, wherein each R is independently as defined above and each L is independently R or a hydroxyl protecting group which is labile in vivo; or R₂, N* and R₄ together form a saturated or unsaturated cyclic, bicyclic or fused ring system as defined hereinafter which may be additionally substituted by —C(O)Y, where Y is as previously defined

and R₃ is X—W-A′-Q-A-, wherein: A′ and A independently are absent or (C₁-C₈)alkylidene, typically (C₁-C₄)alkylidene which may be substituted with one or more substituents R as previously defined;

Q is

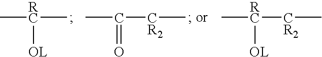

where L and each R, independently of the others, are as previously defined,

and optionally Q and A together, or Q and A′ together, or A′, Q and A together form part of a saturated or unsaturated cyclic, bicyclic or fused ring system as defined hereinafter;

W is absent or is N(R), O or S, wherein R is as previously defined; and X is hydrogen, or X₁, where X₁ is Ra— or RbC(O)— or RbS(O)_{z}—, where z is 1 or 2 and Ra and Rb are independently (C₁-C₁₈)alkyl, typically (C₁-C₁₂)alkyl; (C₃-C₁₈)cycloalkyl, typically (C₃-C₁₂)cycloalkyl; (C₃-C₁₈)cycloalkyl(C₁-C₁₈)alkyl, typically (C₃-C₁₂)cycloalkyl (C₁-C₆)alkyl; heterocyclic; (C₁-C₁₈)alkylheterocyclic, typically (C₁-C₁₂)alkylheterocyclic; heterocyclic(C₆-C₂₄)aryloxy, typically heterocyclic(C₆-C₁₆)aryloxy; (C₁-C₁₈)alkoxy, typically (C₁-C₁₂)alkoxy; (C₁-C₁₈)alkoxy (C₁-C₁₈)alkyl, typically (C₁-C₁₂)alkoxy; (C₁-C₁₂)alkyl; (C₆-C₂₄)aryloxy (C₁-C₁₈)alkyl, typically (C₆-C₁₆)aryloxy(C₁-C₁₂)alkyl; (C₆-C₂₄)aryloxy C₁-C₁₈)alkoxy, typically (C₆-C₁₆)aryloxy(C₁-C₁₂)alkoxy; (C₆-C₂₄)aryl, typically (C₆-C₁₆)aryl; (C₆-C₂₄)aryl(C₁-C₁₈)alkyl, typically (C₆-C₁₆)aryl(C₁-C₁₂)alkyl; (C₆-C₂₄)aryl(C₁-C₁₈)alkylheterocyclic, typically (C₆-C₁₆)aryl; (C₁-C₁₂)alkylheterocyclic; heterocyclicoxy(C₁-C₁₈)alkyl, typically heterocyclicoxy(C₁-C₁₂)alkyl; (C₁-C₁₈)alkylamino, typically (C₁-C₁₂)alkylamino; di(C₁-C₁₈)alkylamino, typically di(C₁-C₁₂)alkylamino; (C₆-C₂₄)arylamino, typically (C₆-C₁₆)arylamino; di(C₆-C₂₄)aryl amino, typically di(C₆-C₁₆)arylamino; (C₇-C₂₅)aralkylamino, typically (C₇-C₁₂)aralkylamino or di(C₇-C₂₅)aralkylamino, typically di(C₇-C₁₂)aralkylamino; any of which may be optionally substituted as hereinbelow defined-or substituted with a group Re, where Re is a group of the formula:

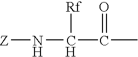

where Z has the meaning of Ra or Rb or is an acylated amino acid, azaamino acid or peptide residue, and Rf is the side-chain of a natural amino acid in which any functional group present is optionally protected;

or X is Re as previously defined,

or X is an optionally protected amino acid, azaamino acid or peptide residue; or

when W is N(R), then X, N and the substituent R on N together may form a saturated or unsaturated cyclic, bicyclic or fused ring system as defined hereinbelow or N, A′ and the substituent R on N together form a saturated or unsaturated cyclic, bicyclic or fused ring system as defined hereinbelow.

In a preferred embodiment the methods comprise administration of a compound of the formula IA:

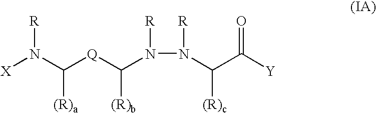

or pharmaceutically acceptable salt thereof;

where X, Q, Y and each R is independently as previously defined, a and b are independently 0 to 4 and c is 0 to 6, or where two R groups, not necessarily vicinal, taken together are —(CHR₁₈)ₘ— where m is 2-8 and R₁₈ has the meaning of R.

In another preferred embodiment, compounds of the general formula (I) have the structure represented by formula (IB):

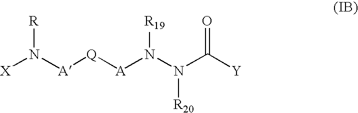

where X, R, A′, Q, A and Y are as previously defined or either or both of A and A′ are absent, and R₁₉ and R₂₀ have the meaning of R or where R₁₉, N*, N and R₂₀ together form a cyclic diazaalkane as previously defined.

In other preferred embodiments, the compounds of general formula (I) have the structure represented by formula (IC) or (ID):

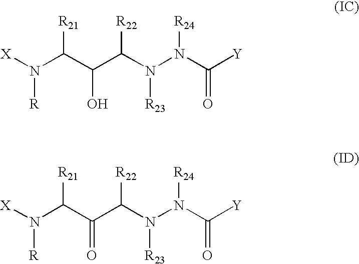

wherein:

R is as defined above;

R₂₁ is hydrogen, optionally substituted (C₁-C₁₂)alkyl; optionally substituted (C₆-C₁₂)aryl; optionally substituted (C₇-C₁₆)aralkyl;

R₂₂ is hydrogen, (C₁-C₈)alkyl; (C₇-C₁₆)aralkyl, or when R₂₁ and R₂₂ taken together are —(CH₂)ₙ—, wherein n is 2 to 8;

R₂₃ is hydrogen; optionally substituted (C₁-C₁₂)alkyl; (C₆-C₁₂)aryl; (C₇-C₁₆)aralkyl; or wherein R₂₂ and R₂₃ taken together are —(CHR₂₅)ₘ—, wherein m is 3-6 and R₂₅ has the meaning of R₁₀;

R₄ is hydrogen; optionally substituted (C₁-C₁₂)alkyl; optionally substituted (C₇-C₁₆)aralkyl; or optionally substituted (C₆ -C₁₂)aryl;

or wherein NR₂₃ and NR₂₄ taken together may be a cyclic diazaalkane as previously defined; and

X and Y are as previously defined.

Preferred compounds for use in the methods of the invention include:

(i) t-butyl 3-isopropyl-3-[(2R or S,3S)-2-hydroxy-3-(phenylmethoxycarbonyl)amino-4-phenylbutyl]carbazate,

(ii) t-butyl 3-isopropyl-3-((2R or S,3S)-2-hydroxy-3-(N-quinaldoyl-L-valyl)amino-4-phenylbutyl]carbazate,

(iii) t-butyl 3-isopropyl-3-[(2R or S,3S)-2-hydroxy-3-(N-quinaldoyl-L-asparaginyl)amino-4-phenylbutyl]carbazate,

(iv) t-butyl 3-isopropyl-3-[(3S)-2-oxo-3-(N-quinaldoyl-L-asparaginyl)amino-4-phenylbuty 1]carbazate,

(v) t-butyl 3-(1-methyl-3-phenylpropen-3-yl)-3-[(2R or S,3S)-2-hydroxy-3-(phenylmethoxycarbonyl)amino-4-phenylbutyl]carbazate,

(vi) t-butyl 3-(1-methyl-3-phenylpropyl)-3-[(2R or S,3S)-2-hydroxy-3-(N-quinaldoyl-L-asparaginyl)amino-4-phenylbutyl]carbazate,

(vii) cis-1,6-3-t-butoxycarbonyl-4-[(2R or S,3S)-2-hydroxy-3-amino-4-phenylbutyl]-3,4-diazabicyclo[4.4.0]decane,

(viii) cis-1,6-3-t-butoxycarbonyl-4-[(2R or S,3S)-2-hydroxy-3-(phenylmethoxycarbonyl)amino-4-phenylbutyl]-diazabicyclo[4.4.0]decane,

(ix) cis-1,6-3-t-butoxycarbonyl-4-[(2R or S,3S)-2-hydroxy-3-(N-quinaldoyl-L-valyl)amino-4-phenylbutyl]-3,4-diazabicyclo[4.4.0]decane

(x) cis-1,6-3-t-butoxycarbonyl-4-[(2R or S,3S)-2-hydroxv-3-(N-(2-pyridyl)methoxycarbonyl)-L-valyl)amino-4-phenylbutyl]-3,4-diazabicyclo[4:4.0]decane

(xi) cis-1,6-3-t-butoxycarbonyl-4-((2R or S,3S)-2-hydroxy-3-(N-quinaldoyl-L-asparaginyl)amino-4-phenylbutyl]-3,4-diazabicyclo[4.4.0]decane,

(xii) cis-1,6-3-t-butoxycarbonyl-4-[(2R or S,3S)-2-hydroxy-3-(N-quinaldoyl-glutaminyl)amino-4-phenylbutyl]-3,4odiazabicyclo[4.4.0]decane,

(xiii) cis-1,6-3-t-butoxycarbonyl-4-[(2R or S,3S)-2-hydroxy-3-(N-quinaldoyl-L-threonyl)amino-4-phenylbutyl]-3,4 -diazabicyclo[4.4.0]decane,

(xiv) 2-t-butoxycarbonyl-3-[(2R or S,3S)-2-hydroxy-3-(phenylmethoxycarbonyl)amino-4-phenylbutyl]-2,3-diazabicyclo[2.2.1]hept-5-ene,

(xv) 2-t-butoxycarbonyl-3-[(2R or S,3S)-2-hydroxy-3-phenylmethoxycarbonyl)amino-4-phenylbutyl]-2,3-diaza-bicyclo[2.2.1]heptane,

(xvi) 2-t-butoxycarbonyl-3-[(2R or S,3S)-2-hydroxy-3-(N-(2-pyridyl)methoxy-L-valyl)amino-4-phenylbutyl]-2,3-diaza-bicyclo[2.2.1 ]heptane,

(xvii) 2-[N-(1S)(2-methyl-1-methoxycarbonylpropyl)carbamoyl]-3-[(2R or S,3S)-2-hydroxy-3-[N-(2-pyridyl)methoxy-L-valyl]amino-4-phenylbutyl]-2,3-diazabicyclo[2.2.1]heptane,

(xviii) 2-t-butoxycarbonyl-3-[(2R or S,3S)-2-hydroxy-3-(N-quinaldoyl-L-asparaginyl)amino-4-phenylbutyl]-2,3-diazabicyclo[2.2.1]heptane,

(ixx) 1-[2-(2-pyridyl)methoxycarbonylamino-]benzoyl-2-[(2R or S,3S)-2-hydroxy-3-(N-quinaldoyl-L-asparaginyl)amino-4-phenylbutyl]-2-isopropylhydrazine,

(xx) 2-t-butoxycarbonyl-3-[(2R or S,3S)-2-hydroxy-3-(N-quinaldoyl-L-asparaginyl)amino-4-phenylbutyl]-1,2,3,4-tetrahydrophthalazine,

(xxi) 1-trimethylacetyl-2-[(2R or S,3S)-2-hydroxy-3-(phenylmethoxycarbonyl)amino-4-phyenylbutyl]-2-isopropyl hydrazine,

(xxii) 1-trimethylacetyl-2-[(2R or S,3S)-2-hydroxy-3-(N-quinaldoyl-L-asparaginyl)amino-4-phenylbutyl]-2-isoprolaylhydrazine,

(xxiii) 1-(t-butylamino)carbonyl-2-[(2R or S,3S)-2-hydroxy-3-(N-quinaldoyl-L-asparaginyl)amino-4-phenylbutyl]-2-isopropylhydrazine,

(xxiv) t-butyl 3-isopropyl-3-((2R or S,38)-2-hydroxy-3-(N-picolinoyl-L-asparaginyl)amino-4-phenylbutyl]carbazate,

(xxv) t-butyl 3-isopropyl-3-[(2R or S,3S)-2-hydroxy-3-(N-(2-pyridyl)methoxycarbonyl-anthraniloyl)amino-4-phenylbutyl]carbazate.

(xxvi) t-butyl 3-benzyl-3-[(2R or S,3S)-2-hydroxy-3-(phenylmethoxycarbonyl)amino-4-phenylbutyl]carbazate,

(xxvii) t-butyl 3-benzyl-3-[(2R or S,3S)-2-hydroxy-3-(N-quinaldoyl-L-asparaginyl)amino-4-phenylbutyl]carbazate,

(xxviii) t-butyl 3-cyclohexyl-3-[(2R or S, 3S)-2-hydroxy-3-(phenyl-methoxycarbonyl)amino-4-phenylbutyl]carbazate,

(xxix) t-butyl 3-cyclohexyl-3-[(2R or S,3S)-2-hydroxy-3-(N-quinaldoyl-L-asparaginyl)amino-4-phenylbutyl]carbazate,

(xxx) t-butyl 3-isopropyl-3-[(2R or S,3S)-2-hydroxy-3-(N-(1-carbamoylmethyl)acryloyl)amino-4-phenylbutyl]carbazate,

(xxxi) t-butyl 3-isopropyl-3-[(2R or S,3S)-2-hydroxy-3-(N-(2(RS)-3-tert-butylthio-2-carbamoyl-methylpropionyl)amino-4-phenylbutyl]carbazate,

(xxxii) t-butyl 3-isopropyl-3-[(2R or S,3S)-2-hydroxy-3-(N-(1-benzoyl-L-asparaginyl)amino-4-phenylbutyl]carbazate,

(xxxiii) 1-t-butyloxycarbonyl-2-[(2R or S,3S)-2-hydroxy-3-(phenylmethoxycarbonyl)amino-4-phenylbutyl]hexahydropyridazine,

(xxxiv) 1-t-butyloxycarbonyl-2-[(2R or S,3S)-2-hydroxy-3-N-quinaldoyl-L-asparaginyl)amino-(4-phenylbutyl]hexahydropyridazine,

(xxxv) cis-1,6-3-t-butoxycarbonyl-4-[(2R or S, 3S)-2-hydroxy-3-(N-quinaldoyl-3-cyano-L-alanyl)amino-4-phenylbutyl]-3,4-diazabicyclo[4,4,0]decane.

The structures of some of the representative compounds for use in the methods of the invention are as follows:

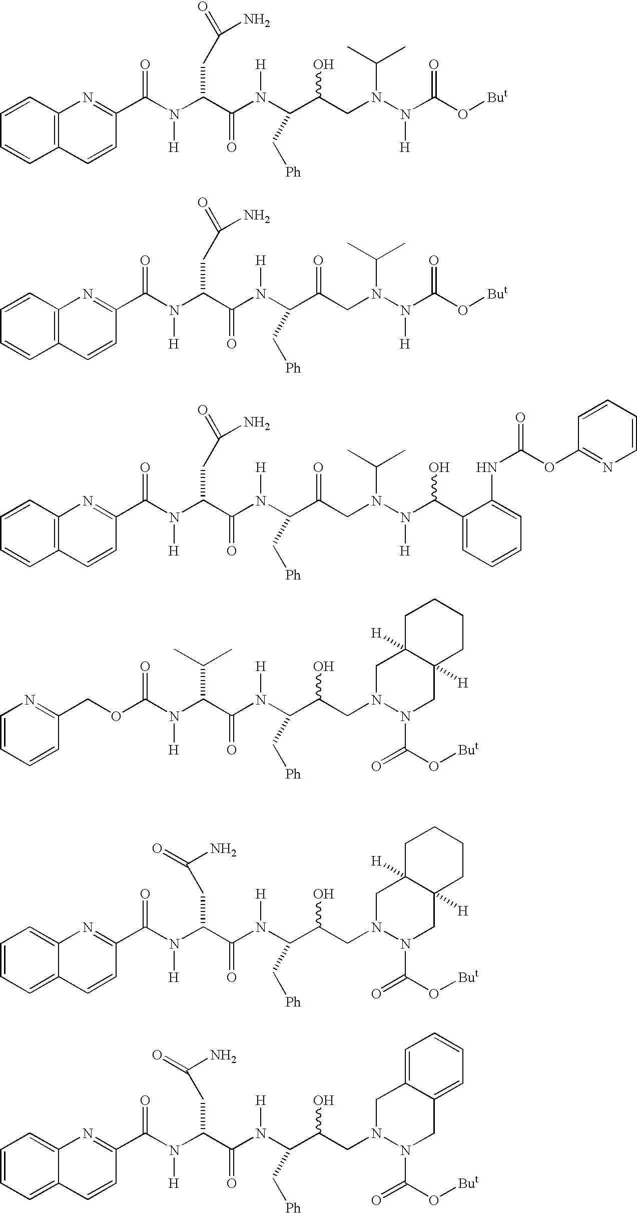

The compounds useful in the methods of the present invention may have asymmetric centers and occur as racemates, racemic mixtures and as individual diastereomers, or enantiomers with all isomeric forms being included in the present invention.

When any variable (e.g., aryl, heterocycle, R¹, R², X, Y, or Z, etc.) occurs more than one time in any constituent or in Formula I, its definition on each occurrence is independent of its definition at every other occurrence. Also, combinations of substituents and/or variables are permissible only if such combinations result in stable compounds.

The compounds of formula (I), (IA), (IB), (IC) or (ID) can exist in optically isomeric forms and the present invention includes within its scope all these forms in all proportions including all diastereoisomers and racemic mixtures.

Compounds employed with the methods of the invention can comprise two R substituents, not necessarily vicinal, taken together that are optionally substituted (C₂-C₁₈)alkylidene, typically (C₂-C₈)alkylidene.

Compounds also employed with the methods of the invention can comprise compounds wherein the Z—NH bond shown is replaced by a modified isosteric bond, such as CH₃—NRa—, RaCH₂—NRa—, CH₃—CHRa—, HCH═CRa—, RaCH═CRa—, HCOCHRa—, RaCOCHRa—, HCHOHCHRa—, RaCHOHCHRa—, HNRaCO—, HCF═CRa—, RaCF═CRa—, RaS(O)—, RaS(O)—, RaP(O)ORa—, RaP(O)(ORa)CH₂—, RaP(O)(ORa)O—, RaP(O)(ORa)S—, wherein each Ra is independently as previously defined.

As used herein, the term “optionally substituted” means that one or more hydrogen atoms may be replaced by a group or groups selected from: —F, —Cl, —Br, —I, —CF₃, —OH, —OR_{IV}, —NH₂, —NHR_{IV}, —NR_{IV}R_{V}, —CN, —NO₂, —SH, —SR_{IV}, —SOR_{IV}, —SO₂R_{IV}, ═O, ═S, ═NOH, ═NOR_{IV}, —NHOH, —NHOR_{IV}, —CHO, where R_{IV} and P_{V} are independently (C₁-C₁₈)alkyl, typically (C₁-C₁₂)alkyl; (C₃-C₁₈)cycloalkyl, typically (C₃-C₁₂)cycloalkyl; (C₃-C₁₈)—cycloalkyl(C₁-C₁₈)alkyl, typically (C₃-C₁₂)cycloalkyl (C₁-C₆)alkyl; (C₆-C₂₄)-aryl, typically (C₆-C₁₆)aryl; (C₇-C₂₅)aralkyl, typically (C₇-C₁₆)aralkyl; (C₂-C₁₈)alkenyl, typically (C₂-C₁₂)alkenyl; (C₈-C₂₆)aralkenyl, typically (C₈C₁₆)aralkenyl; (C₂-C₁₈)alkynyl, typically (C₂-C₁₂)alkynyl; (C₈-C₂₆)-aralkynyl, typically (C₈-C₁₆)aralkynyl; or heterocyclic.

As used herein, the term “alkylidene” refers to optionally unsaturated divalent alkyl radicals. Examples of such radicals are —CH₂—, —CH₂CH₂—, —CH═CH—, —CH₂CH₂CH₂—, —C(═CH₂)CH₂—, —CH₂CH═CH—, —(CH₂)₄—, —CH₂CH₂CH═CH—, —CH₂CH═CHCH₂—, and —(CH₂)ᵣ— where r is 5-8. The term also refers to such radicals in which one or more of the bonds of the radical from part of a cyclic system. Examples of such radicals are groups of the structure

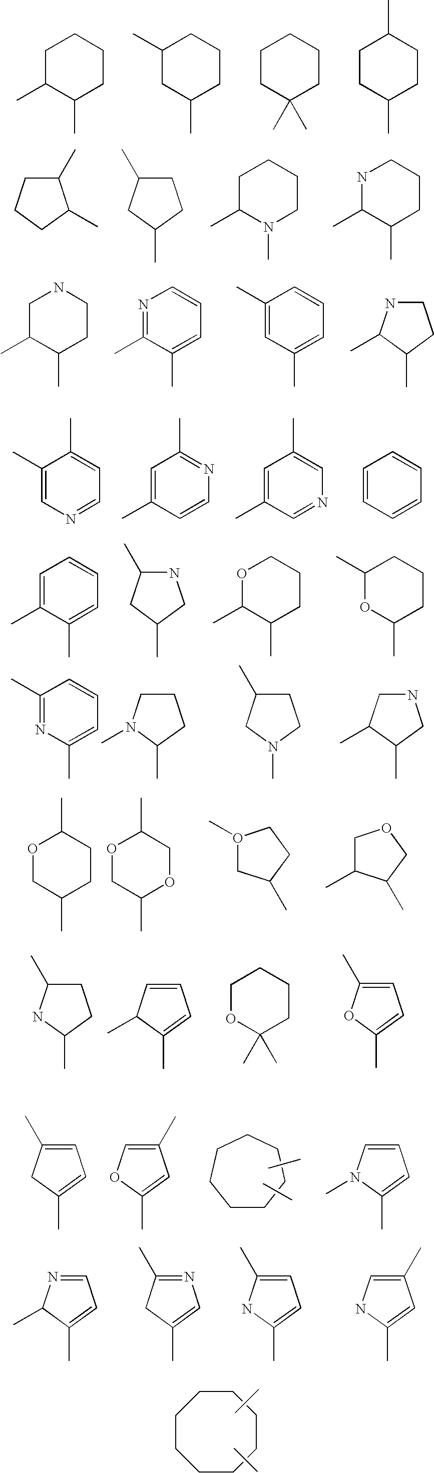

and similar groups wherein any N or O atom is replaced by S.

As used herein, the terms “aralkenyl” and “aralkynyl” refer to alkenyl and alkynyl groups respectively, substituted with one or more aryl groups as previously defined. Examples of such groups are styryl, phenylacetylenyl and 2-phenyl-2-butenyl.

As used herein the term “saturated or unsaturated cyclic, bicyclic or fused ring system” refers to a cyclic system of up to 16 carbon atoms, up to 3 of which may be replaced by O, S or N, which ring system may be substituted with one or more of R, —NH₂, —NHR, —NR₂, —CCOH, —COOL, —CHO, —C(O)R, —CN, halo, —F₃, —OL, —SR, —S(O)R, —S(O)₂R, —CONH₂, —CONHR, —CONR₂, —NHOH, —NHOL, —NO₂, ═O, ═S or —NHNH₂;

wherein each L and R are independently as previously defined. Examples of such ring systems are those cyclic alkylidene groups exemplified above and

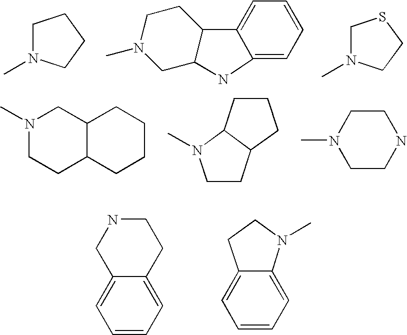

Configurations that result in unstable heterocyclics are not included within the scope of the definition of “heterocyclic” or “saturated or unsaturated cyclic, bicyclic or fused ring system”.

As used herein, the term “alkylheterocyclic” refers to a heterocyclic group as defined above, which is substituted with an alkyl group as defined above.

As used herein, the term “heterocyclic-oxy-alkyl” refers to a group of the formula heterocyclic-O-alkyl, wherein the heterocyclic and alkyl are as defined above.

As used herein, the term “alkoxy” refers to a group of the formula alkyl-O—, wherein the alkyl group is as defined above.

As used herein, the term “aryloxy” refers to a group of the formula aryl-O—, wherein the aryl group is as defined above.

As used herein, the term “alkanoyloxy” refers to a group of the formula alkyl-C(O)O—, wherein the alkyl group is as defined above.

As used herein, the term “amino acid” refers to a synthetic or naturally occurring compound of the formula H₂NCH(R)COOH, wherein R is as defined above.

As used herein, the term “azaamino acid” refers to an amino acid in which the CH(R) group has been replaced by a group —N(R)—, wherein R is as defined above.

Suitable pharmaceutically acceptable salts of the compound of formula (I) include, but are not limited to, salts of pharmaceutically acceptable inorganic acids such as hydrochloric, sulphuric, phosphoric, nitric, carbonic, boric, sulfamic, hydrobromic or hydriodic, or pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic or valeric.

The expression “protected” as used herein is intended to mean that a reactive group such as hydroxyl or amino is substituted by replacing a hydrogen atom of the reactive group in order to protect such groups during synthesis and/or to prevent premature metabolism of the compound of formula (I) after administration to a subject before the compound can reach the desired site of action. Suitable protecting groups for hydroxyl substituents include substituted methyl ethers, for example, methoxymethyl, benzyloxymethyl and the like, vinyl, acyl and carbonate groups. Suitable protecting groups for amino substituents include acyl groups such as acetyl, t-butylacetyl, t-butyloxycarbonyl, benzoyl or carbobenzyloxycarbonyl, benzyloxycarbonyl, pyridinemethoxycarbonyl, quinoline-2-carbonyl or an aminoacyl residue. Protecting groups that can be used with the compounds of formula (I) must be amenable to hydrolytic or metabolic cleavage in vivo.

Preparation of Compounds

The compounds of formula (I) can be prepared by known methods for the synthesis of substituted amines. For example, a compound of the formula

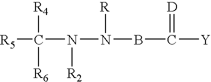

may be prepared by reaction of an amine of the formula

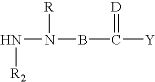

with a substituted alkyl halide of the formula

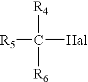

Compounds of formula (IA) may be prepared by reacting an amine of formula

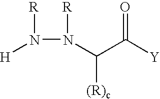

with a halide of formula

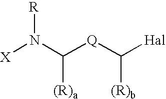

Compounds of formula (IB) may be prepared by reacting an amine of formula

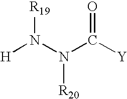

with a halide of formula

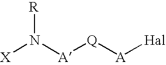

The compounds of formula (IC) can be prepared by reacting a compound of formula (II)

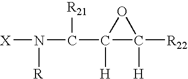

wherein X, R₂₁, R₂₂ and R have the significance given earlier, with a compound of formula (III)

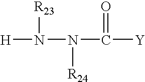

wherein R₂₃, R₂₄ and Y have the significance given earlier.

A compound of formula (ID) may be obtained from a compound of formula (IC) by oxidation in accordance with known methods of oxidative transformations of alcohols to ketones.

A compound of formula (ID) may be also be obtained by reacting a compound of formula (IIa)

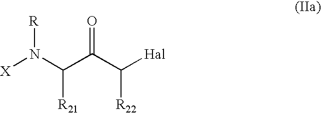

wherein X, R, R₂₁ and R₂₂ are as previously defined and Hal is a group selected from —Cl, —Br, —I or —OS(O)₂R, with a compound of formula (III).

The methods of preparation of compounds of formula (IC) and (ID) may be represented by the following general Schemes 1 to 3. In the Schemes presented herein, the following abbreviations are made:

AA refers to amino acid or amino acid residue; AcCN refers to acetonitrile; BOP refers to benzotriazol-1-yloxytris(dimethylamino)-phosphonium hexafluorophosphate; CBZ refers to carbobenzoxy; CDI refers to N,N′-carbonyldiimidazole; DMF refers to dimethylformamide; DMSO refers to dimethylsulfoxide; HBT refers to 1-hydroxybenzotriazole; Py refers to pyridine; PyxSO₃ refers to the pyridine complex of sulfur trioxide; RT refers to room temperature and L-Val refers to L-valine.

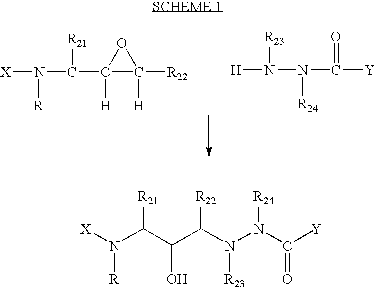

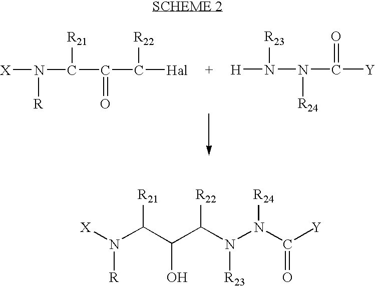

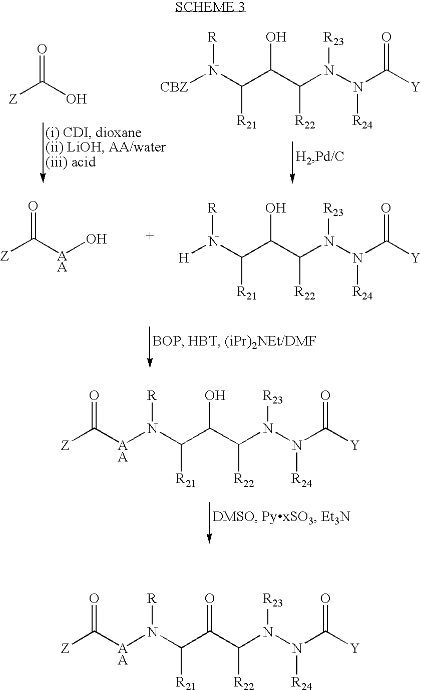

The reaction schemes illustrated can be carried out by generally known methods as exemplified hereinafter. The amino acids or peptide mimics for use in the synthesis of compounds of this invention are generally commercially available or may be prepared by conventional methods of organic chemistry.

Synthetic routes to the intermediates (II), (IIa) and (III) are readily available. The chiral aminoalkylepoxides of formula (II) can be obtained using methods described in the following:

(a) Evans, B. E., et al., J. Org. Chem., 50, 4615-4625 (1985);

(b) Luly, J. R., et al., J. Org. Chem., 52, 1487-1492 (1987);

(c) Handa, B. K., et al., European Patent Application No. 346,847-A2 (1989); and (d) Marshall, G. R., et al., International Patent Application No WO91/08221.

The N-protected aminoalkyl halomethylketones (IIa) are commercially available or can be prepared using methods described in: (e) Rich, et al., J. Med. Chem., 33, 1285-1288 (1990) and reference (d) above.

The hydrazide intermediates (III) can be obtained using known methods such as those described in the following: (g) Dutta, A. S., et al, J. Chem. Soc. Perkin Trans. I, (1975) 1712-1720; (h) Ghali, N. I., et al., J. Org. Chem., 46, 5413-5414 (1981), (i) Gante, J., Synthesis, (1989) 405-413 and (j) Houben-Weyl's Methoden der Organische Chemie, vol. 16a, Part 1, pp 421-855; Georg Thieme Verlag, Stuttgart (1990).

In one aspect, this method of treatment can be used where the disease is Alzheimer's disease.

In another aspect, this method of treatment can help prevent or delay the onset of Alzheimer's disease.

In another aspect, this method of treatment can help slow the progression of Alzheimer's disease.

In another aspect, this method of treatment can be used where the disease is mild cognitive impairment.

In another aspect, this method of treatment can be used where the disease is Down's syndrome.

In another aspect, this method of treatment can be used where the disease is Hereditary Cerebral Hemorrhage with Amyloidosis of the Dutch-Type.

In another aspect, this method of treatment can be used where the disease is cerebral amyloid angiopathy.

In another aspect, this method of treatment can be used where the disease is degenerative dementias.

In another aspect, this method of treatment can be used where the disease is diffuse Lewy body type of Alzheimer's disease.

In another aspect, this method of treatment can treat an existing disease, such as those listed above.

In another aspect, this method of treatment can prevent a disease, such as those listed above, from developing or progressing.

The methods of the invention employ therapeutically effective amounts: for oral administration from about 0.1 mg/day to about 1,000 mg/day; for parenteral, sublingual, intranasal, intrathecal administration from about 0.5 to about 100 mg/day; for depo administration and implants from about 0.5 mg/day to about 50 mg/day; for topical administration from about 0.5 mg/day to about 200 mg/day; for rectal administration from about 0.5 mg to about 500 mg.

In a preferred aspect, the therapeutically effective amounts for oral administration is from about 1 mg/day to about 100 mg/day; and for parenteral administration from about 5 to about 50 mg daily.

In a more preferred aspect, the therapeutically effective amounts for oral administration is from about 5 mg/day to about 50 mg/day.

The present invention also includes the use of a compound of formula (I), or a pharmaceutically acceptable salt thereof for the manufacture of a medicament for use in treating a subject who has, or in preventing a subject from developing, a disease or condition selected from the group consisting of Alzheimer's disease, for helping prevent or delay the onset of Alzheimer's disease, for treating subjects with mild cognitive impairment (MCI) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, frontotemporal dementias with parkinsonism (FTDP), dementia associated with progressive supranuclear palsy, dementia associated with cortical basal degeneration, diffuse Lewy body type of Alzheimer's disease and who is in need of such treatment.

In one aspect, this use of a compound of formula (I) can be employed where the disease is Alzheimer's disease.

In another aspect, this use of a compound of formula (I) can help prevent or delay the onset of Alzheimer's disease.

In another aspect, this use of a compound of formula (I) can help slow the progression of Alzheimer's disease.

In another aspect, this use of a compound of formula (I) can be employed where the disease is mild cognitive impairment.

In another aspect, this use of a compound of formula (I) can be employed where the disease is Down's syndrome.

In another aspect, this use of a compound of formula (I) can be employed where the disease is Hereditary Cerebral Hemorrhage with Amyloidosis of the Dutch-Type.

In another aspect, this use of a compound of formula (I) can be employed where the disease is cerebral amyloid angiopathy.

In another aspect, this use of a compound of formula (I) can be employed where the disease is degenerative dementias.

In another aspect, this use of a compound of formula (I) can be employed where the disease is diffuse Lewy body type of Alzheimer's disease.

In a preferred aspect, this use of a compound of formula (I) is a pharmaceutically acceptable salt of an acid selected from the group consisting of acids hydrochloric, hydrobromic, hydroiodic, nitric, sulfuric, phosphoric, citric, methanesulfonic, CH₃—(CH₂)ₙ—COOH where n is 0 thru 4, HOOC—(CH₂)ₙ—COOH where n is as defined above, HOOC—CH═CH—COOH, and phenyl-COOH.

In another preferred aspect of the invention, the subject or patient is preferably a human subject or patient.

The present invention also includes methods for inhibiting beta-secretase activity, for inhibiting cleavage of amyloid precursor protein (APP), in a reaction mixture, at a site between Met596 and Asp597, numbered for the APP-695 amino acid isotype, or at a corresponding site of an isotype or mutant thereof; for inhibiting production of amyloid beta peptide (A beta) in a cell; for inhibiting the production of beta-amyloid plaque in an animal; and for treating or preventing a disease characterized by beta-amyloid deposits in the brain. These methods each include administration of a therapeutically effective amount of a compound of formula (I), or a pharmaceutically acceptable salt thereof.

The present invention also includes a method for inhibiting beta-secretase activity, including exposing said beta-secretase to an effective inhibitory amount of a compound of formula (I), or a pharmaceutically acceptable salt thereof.

In one aspect, this method includes exposing said beta-secretase to said compound in vitro.

In another aspect, this method includes exposing said beta-secretase to said compound in a cell.

In another aspect, this method includes exposing said beta-secretase to said compound in a cell in an animal.

In another aspect, this method includes exposing said beta-secretase to said compound in a human.

The present invention also includes a method for inhibiting cleavage of amyloid precursor protein (APP), in a reaction mixture, at a site between Met596 and Asp597, numbered for the APP-695 amino acid isotype; or at a corresponding site of an isotype or mutant thereof, including exposing said reaction mixture to an effective inhibitory amount of a compound of formula (I), or a pharmaceutically acceptable salt thereof.

In one aspect, this method employs a cleavage site: between Met652 and Asp653, numbered for the APP-751 isotype; between Met 671 and Asp 672, numbered for the APP-770 isotype; between Leu596 and Asp597 of the APP-695 Swedish Mutation; between Leu652 and Asp653 of the APP-751 Swedish Mutation; or between Leu671-and Asp672 of the APP-770 Swedish Mutation.

In another aspect, this method exposes said reaction mixture in vitro.

In another aspect, this method exposes said reaction mixture in a cell.

In another aspect, this method exposes said reaction mixture in an animal cell.

In another aspect, this method exposes said reaction mixture in a human cell.

The present invention also includes a method for inhibiting production of amyloid beta peptide (A beta) in a cell, including administering to said cell an effective inhibitory amount of a compound of formula (I), or a pharmaceutically acceptable salt thereof.

In an embodiment, this method includes administering to an animal.

In an embodiment, this method includes administering to a human.

The present invention also includes a method for inhibiting the production of beta-amyloid plaque in an animal, including administering to said animal an effective inhibitory amount of a compound of formula (I), or a pharmaceutically acceptable salt thereof.

In one embodiment of this aspect, this method includes administering to a human.

The present invention also includes a method for treating or preventing a disease characterized by beta-amyloid, deposits in the brain including administering to a subject an effective therapeutic amount of a compound of formula (I), or a pharmaceutically acceptable salt thereof.

In one aspect, this method employs a compound at a therapeutic amount in the range of from about 0.1 to about 1000 mg/day.

In another aspect, this method employs a compound at a therapeutic amount in the range of from about 15 to about 1500 mg/day.

In another aspect, this method employs a compound at a therapeutic amount in the range of from about 1 to about 100 mg/day.

In another aspect, this method employs a compound at a therapeutic amount in the range of from about 5 to about 50 mg/day.

In another aspect, this method can be used where said disease is Alzheimer's disease.

In another aspect, this method can be used where said disease is Mild Cognitive Impairment, Down's Syndrome, or Hereditary Cerebral Hemorrhage with Amyloidosis of the Dutch Type.

The present invention also includes a composition including beta-secretase complexed with a compound of formula (I), or a pharmaceutically acceptable salt thereof.

The present invention also includes a method for producing a beta-secretase complex including exposing beta-secretase to a compound of formula (I), or a pharmaceutically acceptable salt thereof, in a reaction mixture under conditions suitable for the production of said complex.

In an embodiment, this method employs exposing in vitro.

In an embodiment, this method employs a reaction mixture that is a cell.

The present invention also includes a component kit including component parts capable of being assembled, in which at least one component part includes a compound of formula (I) enclosed in a container.

In an embodiment, this component kit includes lyophilized compound, and at least one further component part includes a diluent.

The present invention also includes a container kit including a plurality of containers, each container including one or more unit dose of a compound of formula (I), or a pharmaceutically acceptable salt thereof.

In an embodiment, this container kit includes each container adapted for oral delivery and includes a tablet, gel, or capsule.

In an embodiment, this container kit includes each container adapted for parenteral delivery and includes a depot product, syringe, ampoule, or vial.

In an embodiment, this container kit includes each container adapted for topical delivery and includes a patch, medipad, ointment, or cream.

The present invention also includes an agent kit including a compound of formula (I), or a pharmaceutically acceptable salt thereof; and one or more therapeutic agents selected from the group consisting of an antioxidant, an anti-inflammatory, a gamma secretase inhibitor, a neurotrophic agent, an acetyl cholinesterase inhibitor, a statin, an A beta peptide, and an anti-A beta antibody.

The present invention provides compounds, compositions, kits, and methods for inhibiting beta-secretase-mediated cleavage of amyloid precursor protein (APP). More particularly, the compounds, compositions, and methods of the invention are effective to inhibit the production of A beta peptide and to treat or prevent any human or veterinary disease or condition associated with a pathological form of A beta peptide.

The compounds, compositions, and methods of the invention are useful for treating humans who have Alzheimer's Disease (AD), for helping prevent or delay the onset of AD, for treating subjects with mild cognitive impairment (MCI), and preventing or delaying the onset of AD in those subjects who would otherwise be expected to progress from MCI to AD, for treating Down's syndrome, for treating Hereditary Cerebral Hemorrhage with Amyloidosis of the Dutch Type, for treating cerebral beta-amyloid angiopathy and preventing its potential consequences such as single and recurrent lobar hemorrhages, for treating other degenerative dementias, including dementias of mixed vascular and degenerative origin, for treating dementia associated with Parkinson's disease, frontotemporal dementias with parkinsonism (FTDP), dementia associated with progressive supranuclear palsy, dementia associated with cortical basal degeneration, and diffuse Lewy body type AD.

The compounds of the invention possess beta-secretase inhibitory activity. The inhibitory activities of the compounds of the invention are readily demonstrated, for example, using one or more of the assays described herein or known in the art.

The compounds of formula (I) can form salts when reacted with acids. Pharmaceutically acceptable salts are generally preferred over the corresponding compounds of formula (I) since they frequently produce compounds which are usually more water soluble, stable and/or more crystalline. Pharmaceutically acceptable salts are any salt which retains the activity of the parent compound and does not impart any deleterious or undesirable effect on the subject to whom it is administered and in the context in which it is administered. Pharmaceutically acceptable salts include acid addition salts of both inorganic and organic acids. The preferred pharmaceutically acceptable salts include salts of the following acids acetic, aspartic, benzenesulfonic, benzoic, bicarbonic, bisulfuric, bitartaric, butyric, calcium edetate, camsylic, carbonic, chlorobenzoic, citric, edetic, edisylic, estolic, esyl, esylic, formic, fumaric, gluceptic, gluconic, glutamic, glycollylarsanilic, hexamic, hexylresorcinoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, succinic, sulfamic, sulfanilic, sulfonic, sulfuric, tannic, tartaric, teoclic and toluenesulfonic. For other acceptable salts, see Int. J. Pharm., 33, 201-217 (1986) and J. Pharm. Sci., 66(1), 1, (1977).

The present invention provides kits, and methods _or inhibiting beta-secretase enzyme activity and A beta peptide production. Inhibition of beta-secretase enzyme activity halts or reduces the production of A beta from APP and reduces or eliminates the formation of beta-amyloid deposits in the brain.

Methods of the Invention

The compounds of the invention, and pharmaceutically acceptable salts thereof, are useful for treating humans or animals suffering from a condition characterized by a pathological form of beta-amyloid peptide, such as beta-amyloid plaques, and for helping to prevent or delay the onset of such a condition. For example, the compounds are useful for treating Alzheimer's disease, for helping prevent or delay the onset of Alzheimer's disease, for treating subjects with MCI (mild cognitive impairment) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobal hemorrhages, for treating other degenerative dementias, including dementias of mixed vascular and degenerative origin, dementia associated with Parkinson's disease, frontotemporal dementias with parkinsonism (FTDP), dementia associated with progressive supranuclear palsy, dementia associated with cortical basal degeneration, and diffuse Lewy body type Alzheimer's disease. The compounds and compositions of the invention are particularly useful for treating, preventing, or slowing the progression of Alzheimer's disease. When treating or preventing these diseases, the compounds of the invention can either be used individually or in combination, as is best for the subject or subject.

With regard to these diseases, the term “treating” means that compounds of the invention can be used in humans with existing disease. The compounds of the invention will not necessarily cure the subject who has the disease but will delay or slow the progression or prevent further progression of the disease thereby giving the individual a more useful life span.

The term “preventing” means that that if the compounds of the invention are administered to those who do not now have the disease but who would normally develop the disease or be at increased risk for the disease, they will not develop the disease. In addition, “preventing” also includes delaying the development of the disease in an individual who will ultimately develop the disease or would be at risk for the disease due to age, familial history, genetic or chromosomal abnormalities, and/or due to the presence of one or more biological markers for the disease, such as a known genetic mutation of APP or APP cleavage products in brain tissues or fluids. By delaying the onset of the disease, compounds of the invention have prevented the individual from getting the disease during the period in which the individual would normally have gotten the disease or reduce the rate of development of the disease or some of its effects but for the administration of compounds of the invention up to the time the individual ultimately gets the disease. Preventing also includes administration of the compounds of the invention to those individuals thought to be predisposed to the disease.

In a preferred aspect, the compounds of the invention are useful for slowing the progression of disease symptoms.

Compositions are provided that contain therapeutically effective amounts of the compounds of the invention. The compounds are preferably formulated into suitable pharmaceutical preparations such as tablets, capsules, or elixirs for oral administration or in sterile solutions or suspensions for parenteral administration. Typically the compounds described above are formulated into pharmaceutical compositions using techniques and procedures well known in the art.

About 1 to 500 mg of a compound or mixture of compounds of the invention or a physiologically acceptable salt or ester is compounded with a physiologically acceptable vehicle, carrier, excipient, binder, preservative, stabilizer, flavor, etc., in a unit dosage form as called for by accepted pharmaceutical practice. The amount of active substance in those compositions or preparations is such that a suitable dosage in the range indicated is obtained. The compositions are preferably formulated in a unit dosage form, each dosage containing from about 2 to about 100 mg, more preferably about 10 to about 30 mg of the active ingredient. The term “unit dosage from” refers to physically discrete units suitable as unitary dosages for human subjects and other mammals, each unit containing a predetermined quantity of active material calculated to produce the desired therapeutic effect, in association with a suitable pharmaceutical excipient.

To prepare compositions, one or more compounds of the invention are mixed with a suitable pharmaceutically acceptable carrier. Upon mixing or addition of the compound(s), the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. The effective concentration is sufficient for lessening or ameliorating at least one symptom of the disease, disorder, or condition treated and may be empirically determined.

Pharmaceutical carriers or vehicles suitable for administration of the compounds provided herein include any such carriers known to those skilled in the art to be suitable for the particular mode of administration. In addition, the active materials can also be mixed with other active materials that do not impair the desired action, or with materials that supplement the desired action, or have another action. The compounds may be formulated as the sole pharmaceutically active ingredient in the composition or may be combined with other active ingredients.

Where the compounds exhibit insufficient solubility, methods for solubilizing may be used. Such methods are known and include, but are not limited to, using cosolvents such as dimethylsulfoxide (DMSO), using surfactants such as Tween®, and dissolution in aqueous sodium bicarbonate. Derivatives of the compounds, such as salts or prodrugs may also be used in formulating effective pharmaceutical compositions.

The concentration of the compound is effective for delivery of an amount upon administration that lessens or ameliorates at least one symptom of the disorder for which the compound is administered. Typically, the compositions are formulated for single dosage administration.

The compounds of the invention may be prepared with carriers that protect them against rapid elimination from the body, such as time-release formulations or coatings. Such carriers include controlled release formulations, such as, but not limited to, microencapsulated delivery systems. The active compound is included in the pharmaceutically acceptable carrier in an amount sufficient to exert a therapeutically useful effect in the absence of undesirable side effects on the subject treated. The therapeutically effective concentration may be determined empirically by testing the compounds in known in vitro and in vivo model systems for the treated disorder.

The compounds and compositions of the invention can be enclosed in multiple or single dose containers. The enclosed compounds and compositions can be provided in kits, for example, including component parts that can be assembled for use. For example, a compound inhibitor in lyophilized form and a suitable diluent may be provided as separated components for combination prior to use. A kit may include a compound inhibitor and a second therapeutic agent for co-administration. The inhibitor and second therapeutic agent may be provided as separate component parts. A kit may include a plurality of containers, each container holding one or more unit dose of the compound of the invention. The containers are preferably adapted for the desired mode of administration, including, but not limited to tablets, gel capsules, sustained-release capsules, and the like for oral administration; depot products, pre-filled syringes, ampoules, vials, and the like for parenteral administration; and patches, medipads, creams, and the like for topical administration.

The concentration of active compound in the drug composition will depend on absorption, inactivation, and excretion rates of the active compound, the dosage schedule, and amount administered as well as other factors known to those of skill in the art.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

If oral administration is desired, the compound should be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition.

The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

The active materials can also be mixed with other active materials that do not impair the desired action, or with materials that supplement the desired action.

Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers. These may be prepared according to methods known for example, as described in U.S. Pat. No. 4,522,811.

The active compounds may be prepared with carriers that protect the compound against rapid elimination from the body, such as time-release formulations or coatings. Such carriers include controlled release formulations, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, and the like. Methods for preparation of such formulations are known to those skilled in the art.

The compounds of the invention can be administered orally, parenterally (IV, IM, depo-IM, SQ, and depo-SQ), sublingually, intranasally (inhalation), intrathecally, topically, or rectally. Dosage forms known to those skilled in the art are suitable for delivery of the compounds of the invention.

Compounds of the invention may be administered enterally or parenterally. When administered orally, compounds of the invention can be administered in usual dosage forms for oral administration as is well known to those skilled in the art. These dosage forms include the usual solid unit dosage forms of tablets and capsules as well as liquid dosage forms such as solutions, suspensions, and elixirs. When the solid dosage forms are used, it is preferred that they be of the sustained release type so that the compounds of the invention need to be administered only once or twice daily.

The oral dosage forms are administered to the subject 1, 2, 3, or 4 times daily. It is preferred that the compounds of the invention be administered either three or fewer times, more preferably once or twice daily. Hence, it is preferred that the compounds of the invention be administered in oral dosage form. It is preferred that whatever oral dosage form is used, that it be designed so as to protect the compounds of the invention from the acidic environment of the stomach. Enteric coated tablets are well known to those skilled in the art. In addition, capsules filled with small spheres each coated to protect from the acidic stomach, are also well known to those skilled in the art.

When administered orally, an administered amount therapeutically effective to inhibit beta-secretase activity, to inhibit A beta production, to inhibit A beta deposition, or to treat or prevent AD is from about 0.1 mg/day to about 1,000 mg/day. It is preferred that the oral dosage is from about 1 mg/day to about 100 mg/day. It is more preferred that the oral dosage is from about 5 mg/day to about 50 mg/day. It is understood that while a subject may be started at one dose, that dose may be varied over time as the subject's condition changes.

Compounds of the invention may also be advantageously delivered in a nano crystal dispersion formulation. Preparation of such formulations is described, for example, in U.S. Pat. No. 5,145,684. Nano crystalline dispersions of HIV protease inhibitors and their method of use are described in U.S. Pat. No. 6,045,829. The nano crystalline formulations typically afford greater bioavailability of drug compounds.

The compounds of the invention can be administered parenterally, for example, by IV, IM, depo-IM, SC, or depo-SC. When administered parenterally, a therapeutically effective amount of about 0.5 to about 100 mg/day, preferably from about 5 to about 50 mg daily should be delivered. When a depot formulation is used for injection once a month or once every two weeks, the dose should be about 0.5 mg/day to about 50 mg/day, or a monthly dose of from about 15 mg to about 1,500 mg. In part because of the forgetfulness of the subjects with Alzheimer's disease, it is preferred that the parenteral dosage form be a depo formulation.

The compounds of the invention can be administered sublingually. When given sublingually, the compounds of the invention should be given one to four times daily in the amounts described above for IM administration.

The compounds of the invention can be administered intranasally. When given by this route, the appropriate dosage forms are a nasal spray or dry powder, as is known to those skilled in the art. The dosage of the compounds of the invention for intranasal administration is the amount described above for IM administration.

The compounds of the invention can be administered intrathecally. When given by this route the appropriate dosage form can be a parenteral dosage form as is known to those skilled in the art. The dosage of the compounds of the invention for intrathecal administration is the amount described above for IM administration.

The compounds of the invention can be administered topically. When given by this route, the appropriate dosage form is a cream, ointment, or patch. Because of the amount of the compounds of the invention to be administered, the patch is preferred. When administered topically, the dosage is from about 0.5 mg/day to about 200 mg/day. Because the amount that can be delivered by a patch is limited, two or more patches may be used. The number and size of the patch is not important, what is important is that a therapeutically effective amount of the compounds of the invention be delivered as is known to those skilled in the art. The compounds of the invention can be administered rectally by suppository as is known to those skilled in the art. When administered by suppository, the therapeutically effective amount is from about 0.5 mg to about 500 mg. The compounds of the invention can be administered by implants as is known to those skilled in the art. When administering a compound of the invention by implant, the therapeutically effective amount is the amount described above for depot administration.

The invention here is the new compounds of the invention and new methods of using the compounds of the invention. Given a particular compound of the invention and a desired dosage form, one skilled in the art would know how to prepare and administer the appropriate dosage form.

The compounds of the invention are used in the same manner, by the same routes of administration, using the same pharmaceutical dosage forms, and at the same dosing schedule as described above, for preventing disease or treating subjects with MCI (mild cognitive impairment) and preventing or delaying the onset of Alzheimer's disease in those who would progress from MCI to AD, for treating or preventing Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, frontotemporal dementias with parkinsonism (FTDP), dementia associated with progressive supranuclear palsy, dementia associated with cortical basal degeneration, and diffuse Lewy body type of Alzheimer's disease.

The compounds of the invention can be used with each other or with other agents used to treat or prevent the conditions listed above. Such agents include gamma-secretase inhibitors, anti-amyloid vaccines and pharmaceutical agents such as donepezil hydrochloride (ARICEPT Tablets), tacrine hydrochloride (COGNEX Capsules) or other acetylcholine esterase inhibitors and with direct or indirectneurotropic agents of the future.

In addition, the compounds of the invention can also be used with inhibitors of P-glycoproten (P-gp). The use of P-gp inhibitors is known to those skilled in the art. See for example, Cancer Research, 53, 4595-4602 (1993), Clin. Cancer Res., 2, 7-12 (1996), Cancer Research, 56, 4171-4179 (1996), International Publications WO99/64001 and WO01/10387. The important thing is that the blood level of the P-gp inhibitor be such that it exerts its effect in inhibiting P-gp from decreasing brain blood levels of the compounds of the invention. To that end the P-gp inhibitor and the compounds of the invention can be administered at the same time, by the same or different route of administration, or at different times. The important thing is not the time of administration but having an effective blood level of the P-gp inhibitor.

Suitable P-gp inhibitors include cyclosporin A, verapamil, tamoxifen, quinidine, Vitamin E-TGPS, ritonavir, megestrol acetate, progesterone, rapamycin, 10,11-methanodibenzosuberane, phenothiazines, acridine derivatives such as GF120918, FK506, VX-710, LY335979, PSC-833, GF-102,918 and other steroids. It Is to be understood that additional agents will be found that do the same function and are also considered to be useful.

The P-gp inhibitors can be administered orally, parenterally, (IV, IM, IM-depo, SQ, SQ-depo), topically, sublingually, rectally, intranasally, intrathecally and by implant.

The therapeutically effective amount of the P-gp inhibitors is from about 0.1 to about 300 mg/kg/day, preferably about 0.1 to about 150 mg/kg daily. It is understood that while a subject may be started on one dose, that dose may have to be varied over time as the subject's condition changes.

When administered orally, the P-gp inhibitors can be administered in usual dosage forms for oral administration as is known to those skilled in the art. These dosage forms include the usual solid unit dosage forms of tablets and capsules as well as liquid dosage forms such as solutions, suspensions and elixirs. When the solid dosage forms are used, it is preferred that they be of the sustained release type so that the P-gp inhibitors need to be administered only once or twice daily. The oral dosage forms are administered to the subject one through four times daily. It is preferred that the P-gp inhibitors be administered either three or fewer times a day, more preferably once or twice daily. Hence, it is preferred that the P-gp inhibitors be administered in solid dosage form and further it is preferred that the solid dosage form be a sustained release form which permits once or twice daily dosing. It is preferred that what ever dosage form is used, that it be designed so as to protect the P-gp inhibitors from the acidic environment of the stomach. Enteric coated tablets are well known to those skilled in the art. In addition, capsules filled with small spheres each coated to protect from the acidic stomach, are also well known to those skilled in the art.

In addition, the P-gp inhibitors can be administered parenterally. When administered parenterally they can be administered IV, IM, depo-IM, SQ or depo-SQ. The P-gp inhibitors can be given sublingually. When given sublingually, the P-gp inhibitors should be given one thru four times daily in the same amount as for IM administration.

The P-gp inhibitors can be given intranasally. When given by this route of administration, the appropriate dosage forms are a nasal spray or dry powder as is known to those skilled in the art. The dosage of the P-gp inhibitors for intranasal administration is the same as for IM administration.

The P-gp inhibitors can be given intrathecally. When given by this route of administration the appropriate dosage form can be a parenteral dosage form as is known to those skilled in the art.

The P-gp inhibitors can be given topically. When given by this route of administration, the appropriate dosage form is a cream, ointment or patch. Because of the amount of the P-gp inhibitors needed to be administered the path is preferred. However, the amount that can be delivered by a patch is limited. Therefore, two or more patches may be required. The number and size of the patch is not important, what is important is that a therapeutically effective amount of the P-gp inhibitors be delivered as is known to those skilled in the art. The P-gp inhibitors can be administered rectally by suppository as is known to those skilled in the art.

The P-gp inhibitors can be administered by implants as is known to those skilled in the art.

There is nothing novel about the route of administration nor the dosage forms for administering the P-gp inhibitors. Given a particular P-gp inhibitor, and a desired dosage form, one skilled in the art would know how to prepare the appropriate dosage form for the P-gp inhibitor.

The compounds employed in the methods of the invention can be used in combination, with each other or with other therapeutic agents or approaches used to treat or prevent the conditions listed above. Such agents or approaches include: acetylcholine esterase inhibitors such as tacrine (tetrahydroaminoacridine, marketed as COGNEX®), donepezil hydrochloride, (marketed as Aricept® and rivastigmine (marketed as Exelon®); gamma-secretase inhibitors; anti-inflammatory agents such as cyclooxygenase II inhibitors; anti-oxidants such as Vitamin E and ginkolides; immunological approaches, such as, for example, immunization with A beta peptide or administration of anti-A beta peptide antibodies; statins; and direct or indirect neurotropic agents such as Cerebrolysin®, AIT-082 (Emilieu, 2000, Arch. Neurol. 57:454), and other neurotrop_agents of the future.

It should be apparent to one skilled in the art that the exact dosage and frequency of administration will depend on the particular compounds employed in the methods of the invention administered, the particular condition being treated, the severity of the condition being treated, the age, weight, general physical condition of the particular subject, and other medication the individual may be taking as is well known to administering physicians who are skilled in this art.

Inhibition of APP Cleavage

The compounds of the invention inhibit cleavage of APP between Met595 and Asp596 numbered for the APP695 isoform, or a mutant thereof, or at a corresponding site of a different isoform, such as APP751 or APP770, or a mutant thereof (sometimes referred to as the “beta secretase site”). While not wishing to be bound by a particular theory, inhibition of beta-secretase activity is thought to inhibit production of beta amyloid peptide (A beta). Inhibitory activity is demonstrated in one of a variety of inhibition assays, whereby cleavage of an APP substrate in the presence of a beta-secretase enzyme is analyzed in the presence of the inhibitory compound, under conditions normally sufficient to result in cleavage at the beta-secretase cleavage site. Reduction of APP cleavage at the beta-secretase cleavage site compared with an untreated or inactive control is correlated with inhibitory activity. Assay systems that can be used to demonstrate efficacy of the compound inhibitors of the invention are known. Representative assay systems are described, for example, in U.S. Pat. Nos. 5,942,400, 5,744,346, as well as in the Examples below.

The enzymatic activity of beta-secretase and the production of A beta can be analyzed in vitro or in vivo, using natural, mutated, and/or synthetic APP substrates, natural, mutates and/or synthetic enzyme, and the test compound. The analysis may involve primary or secondary cells expressing native, mutant, and/or synthetic APP and enzyme, animal models expressing native APP and enzyme, or may utilize transgenic animal models expressing the substrate and enzyme. Detection of enzymatic activity can be by analysis of one or more of the cleavage products, for example, by immunoassay, fluorometric or chromogenic assay, HPLC, or other means of detection. Inhibitory compounds are determined as those having the ability to decrease the amount of beta-secretase cleavage product produced in comparison to a control, where beta-secretase mediated cleavage in the reaction system is observed and measured in the absence of inhibitory compounds.

Beta-Secretase

Various forms of beta-secretase enzyme are known, and are available and useful for assay of enzyme activity and inhibition of enzyme activity. These include native, recombinant, and synthetic forms of the enzyme. Human beta-secretase is known as Beta Site APP Cleaving Enzyme (BACE), Asp2, and memapsin 2, and has been characterized, for example, in U.S. Pat. No. 5,744,346 and published PCT patent applications WO98/22597, WO00/03819, WO01/23533, and WO00/17369, as well as in literature publications (Hussain et al., 1999, Mol. Cell. Neurosci. 14:419-427; Vassar et al., 1999, Science 286:735-741; Yan et al., 1999, Nature 402:533-537; Sinha et al., 1999, Nature 40:537-540; and Lin et al., 2000, PNAS USA 97:1456-1460) Synthetic forms of the enzyme have also been described (WO98/22597 and WO00/17369). Beta-secretase can be extracted and purified from human brain tissue and can be produced in cells, for example mammalian cells expressing recombinant enzyme.

Preferred methods employ compounds that are effective to inhibit 50% of beta-secretase enzymatic activity at a concentration of less than about 50 micromolar, preferably at a concentration of less than about 10 micromolar, more preferably less than about 1 micromolar, and most preferably less than about 10 nanomolar.

APP Substrate

Assays that demonstrate inhibition of beta-secretase-mediated cleavage of APP can utilize any of the known forms of APP, including the 695 amino acid “normal” isotype described by Kang et al., 1987, Nature 325:733-6, the 770 amino acid isotype described by Kitaguchi et. al., 1981, Nature 331:530-532, and variants such as the Swedish Mutation (KM670-1NL) (APP-SW), the London Mutation (V7176F), and others. See, for example, U.S. Pat. No. 5,766,846 and also Hardy, 1992, Nature Genet. 1:233-234, for a review of known variant mutations. Additional useful substrates include the dibasic amino acid modification, APP-KK disclosed, for example, in WO 00/17369, fragments of APP, and synthetic peptides containing the beta-secretase cleavage site, wild type (WT) or mutated form, e.g., SW, as described, for example, in U.S. Pat. No 5,942,400 and WO00/03819.

The APP substrate contains the beta-secretase cleavage site of APP (KM-DA or NL-DA) for example, a complete APP peptide or variant, an APP fragment, a recombinant or synthetic APP, or a fusion peptide. Preferably, the fusion peptide includes the beta-secretase cleavage site fused to a peptide having a moiety useful for enzymatic assay, for example, having isolation and/or detection properties. A useful moiety may be an antigenic epitope for antibody binding, a label or other detection moiety, a binding substrate, and the like.

Antibodies

Products characteristic of APP cleavage can be measured by immunoassay using various antibodies, as described, for example, in Pirttila et al., 1999, Neuro. Lett. 249:21-4, and in U.S. Pat. No. 5,612,486. Useful antibodies to detect A beta include, for example, the monoclonal antibody 6E10 (Senetek, St. Louis, Mo.) that specifically recognizes an epitope on amino acids 1-16 of the A beta peptide; antibodies 162 and 164 (New York State Institute for Basic Research, Staten Island, N.Y.) that are specific for human A beta 1-40 and 1-42, respectively; and antibodies that recognize the junction region of beta-amyloid peptide, the site between residues 16 and 17, as described in U.S. Pat. No. 5,593,846. Antibodies raised against a synthetic peptide of residues 591 to 596 of APP and SW192 antibody raised against 590-596 of the Swedish mutation are also useful in immunoassay of APP and its cleavage products, as described in U.S. Pat. Nos. 5,604,102 and 5,721,130.

Assay Systems

Assays for determining APP cleavage at the beta-secretase cleavage site are well known in the art. Exemplary assays, are described, for example, in U.S. Pat. Nos. 5,744,346 and 5,942,400, and described in the Examples below.

Cell Free Assays

Exemplary assays that can be used to demonstrate the inhibitory activity of the compounds of the invention are described, for example, in WO00/17369, WO 00/03819, and U.S. Pat. Nos. 5,942,400 and 5,744,346. Such assays can be performed in cell-free incubations or in cellular incubations using cells expressing a beta-secretase and an APP substrate having a beta-secretase cleavage site.

An APP substrate containing the beta-secretase cleavage site of APP, for example, a complete APP or variant, an APP fragment, or a recombinant or synthetic APP substrate containing the amino acid sequence: KM-DA or NL-DA, is incubated in the presence of beta-secretase enzyme, a fragment thereof, or a synthetic or recombinant polypeptide variant having beta-secretase activity and effective to cleave the beta-secretase cleavage site of APP, under incubation conditions suitable for the cleavage activity of the enzyme. Suitable substrates optionally include derivatives that may be fusion proteins or peptides that contain the substrate peptide and a modification useful to facilitate the purification or detection of the peptide or its beta-secretase cleavage products. Useful modifications include the insertion of a known antigenic epitope for antibody binding; the linking of a label or detectable moiety, the linking of a binding substrate, and the like.

Suitable incubation conditions for a cell-free in vitro assay include, for example: approximately 200 nanomolar to 10 micromolar substrate, approximately 10 to 200 picomolar enzyme, and approximately 0.1 nanomolar to 10 micromolar inhibitor compound, in aqueous solution, at an approximate pH of 4-7, at approximately 37 degrees C., for a time period of approximately 10 minutes to 3 hours. These incubation conditions are exemplary only, and can be varied as required for the particular assay components and/or desired measurement system. Optimization of the incubation conditions for the particular assay components should account for the specific beta-secretase enzyme used and its pH optimum, any additional enzymes and/or markers that might be used in the assay, and the like. Such optimization is routine and will not require undue experimentation.

One useful assay utilizes a fusion peptide having maltose binding protein (MBP) fused to the C-terminal 125 amino acids of APP-SW. The MBP portion is captured on an assay substrate by anti-MBP capture antibody. Incubation of the captured fusion protein in the presence of beta-secretase results in cleavage or the substrate at the beta-secretase cleavage site. Analysis of the cleavage activity can be, for example, by immunoassay of cleavage products. One such immunoassay detects a unique epitope exposed at the carboxy terminus of the cleaved fusion protein, for example, using the antibody SW192. This assay is described, for example, in U.S. Pat. No 5,942,400.

Cellular Assay

Numerous cell-based assays can be used to analyze beta-secretase activity and/or processing of APP to release A beta. Contact of an APP substrate with a beta-secretase enzyme within the cell and in the presence or absence of a compound inhibitor of the invention can be used to demonstrate beta-secretase inhibitory activity of the compound. Preferably, assay in the presence of a useful inhibitory compound provides at least about 30%, most preferably at least about 50% inhibition of the enzymatic activity, as compared with a non-inhibited control.

In one embodiment, cells that naturally express beta-secretase are used. Alternatively, cells are modified to express a recombinant beta-secretase or synthetic variant enzyme as discussed above. The APP substrate may be added to the culture medium and is preferably expressed in the cells. Cells that naturally express APP, variant or mutant forms of APP, or cells transformed to express an isoform of APP, mutant or variant APP, recombinant or synthetic APP, APP fragment, or synthetic APP peptide or fusion protein containing the beta-secretase APP cleavage site can be used, provided that the expressed APP is permitted to contact the enzyme and enzymatic cleavage activity can be analyzed.

Human cell lines that normally process A beta from APP provide a useful means to assay inhibitory activities of the compounds of the invention. Production and release of A beta and/or other cleavage products into the culture medium can be measured, for example by immunoassay, such as Western blot or enzyme-linked immunoassay (EIA) such as by ELISA.

Cells expressing an APP substrate and an active beta-secretase can be incubated in the presence of a compound inhibitor to demonstrate inhibition of enzymatic activity as compared with a control. Activity of beta-secretase can be measured by analysis of one or more cleavage products of the APP substrate. For example, inhibition of beta-secretase activity against the substrate APP would be expected to decrease release of specific beta-secretase induced APP cleavage products such as A beta.

Although both neural and non-neural cells process and release A beta, levels of endogenous beta-secretase activity are low and often difficult to detect by EIA. The use of cell types known to have enhanced beta-secretase activity, enhanced processing of APP to A beta, and/or enhanced production of A beta are therefore preferred. For example, transfection of cells with the Swedish Mutant form of APP (APP-SW); with APP-KK; or with APP-SW-KK provides cells having enhanced beta-secretase activity and producing amounts of A beta that can be readily measured.

In such assays, for example, the cells expressing APP and beta-secretase are incubated in a culture medium under conditions suitable for beta-secretase enzymatic activity at its cleavage site on the APP substrate. On exposure of the cells to the compound inhibitor, the amount of A beta released into t medium and/or the amount of CTF99 fragments of APP in the cell lysates is reduced as compared with the control. The cleavage products of APP can be analyzed, for example, by immune reactions with specific antibodies, as discussed above.

Preferred cells for analysis of beta-secretase activity include primary human neuronal cells, primary transgenic animal neuronal cells where the transgene is APP, and other cells such as those of a stable 293 cell line expressing APP, for example, APP-SW.

In vivo Assays: Animal Models

Various animal models can be used to analyze beta-secretase activity and/or processing of APP to release A beta, as described above. For example, transgenic animals expressing APP substrate and beta-secretase enzyme can be used to demonstrate inhibitory activity of the compounds of the invention. Certain transgenic animal models have been described, for example, in U.S. Pat. Nos.: 5,877,399; 5,612,486; 5,387,742; 5,720,936; 5,850,003; 5,877,015,, and 5,811,633, and in Ganes et al., 1995, Nature 373:523. Preferred are animals that exhibit characteristics associated with the pathophysiology of AD. Administration of the compound inhibitors of the invention to the transgenic mice described herein provides an alternative method for demonstrating the inhibitory activity of the compounds. Administration of the compounds in a pharmaceutically effective carrier and via an administrative route that reaches the target tissue in an appropriate therapeutic amount is also preferred.

Inhibition of beta-secretase mediated cleavage of APP at the beta-secretase cleavage site and of A beta release can be analyzed in these animals by measure of cleavage fragments in the animal's body fluids such as cerebral fluid or tissues. Analysis of brain tissues for A beta deposits or plagues is preferred.

On contacting an APP substrate with a beta-secretase enzyme in the presence of an inhibitory compound of the invention and under conditions sufficient to permit enzymatic mediated cleavage of APP and/or release of A beta from the substrate, the compounds of the invention are effective to reduce beta-secretase-mediated cleavage of APP at the beta-secretase cleavage site and/or effective to reduce released amounts of A beta. Where such contacting is the administration of the inhibitory compounds of the invention to an animal model, for example, as described above, the compounds are effective to reduce A beta deposition in brain tissues of the animal, and to reduce the number and/or size of beta amyloid plaques. Where such administration is to a human subject, the compounds are effective to inhibit or slow the progression of disease characterized by enhanced amounts of A beta, to slow the progression of AD in the, and/or to prevent onset or development of AD in a subject at risk for the disease.

Unless defined otherwise, all scientific and technical terms used herein have the same meaning as commonly understood by one of skill in the art to which this invention belongs. All patents and publications referred to herein are hereby incorporated by reference for all purposes.

Definitions

Unless defined otherwise, all scientific and technical terms used herein have the same meaning as commonly understood by one of skill in the art to which this invention belongs.

All patents and publications referred to herein are hereby incorporated by reference for all purposes.

APP, amyloid precursor protein, is defined as any APP polypeptide, including APP variants, mutations, and isoforms, for example, as disclosed in U.S. Pat. No. 5,766,846.

A beta, amyloid beta peptide, is defined as any peptide resulting from beta-secretase mediated cleavage of APP, including peptides of 39, 40, 41, 42, and 43 amino acids, and extending from the beta-secretase cleavage site to amino acids 39, 40, 41, 42, or 43.

Beta-secretase (BACE1, Asp2, Memapsin 2) is an aspartyl protease that mediates cleavage of APP at the amino-terminal edge of A beta. Human beta-secretase is described, for example, in WO00/17369.

Pharmaceutically acceptable refers to those properties and/or substances that are acceptable to the subject from a pharmacological/toxicological point of view and to the manufacturing pharmaceutical chemist from a physical/chemical point of view regarding composition, formulation, stability, subject's acceptance and bioavailability.

A therapeutically effective amount is defined as an amount effective to reduce or lessen at least one symptom of the disease being treated or to reduce or delay onset of one or more clinical markers or symptoms of the disease.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

As noted above, depending on whether asymmetric carbon atoms are present, the compounds of the invention can be present as mixtures of isomers, especially as racemates, or in the form of pure isomers, especially optical antipodes.

Salts of compounds having salt-forming groups are especially acid addition salts, salts with bases or, where several salt-forming groups are present, can also be mixed salts or internal salts.

Salts are especially the pharmaceutically acceptable or non-toxic salts of compounds of formula I.

Such salts are formed, for example, by compounds of formula I having an acid group, for example a carboxy group or a sulfa group, and are, for example, salts thereof with suitable bases, such as non-toxic metal salts derived from metals of groups Ia, Ib, IIa and IIb of the Periodic Table of the Elements, for example alkali metal salts, especially lithium, sodium or potassium salts, or alkaline earth metal salts, for example magnesium or calcium salts, also zinc salts or ammonium salts, as well as salts formed with organic amines, such as unsubstituted or hydroxy-substituted mono-, di- or tri-alkylamines, especially mono-, di- or tri-lower alkylamines, or with quaternary ammonium bases, for example with methyl-, ethyl-, diethyl- or triethyl-amine, mono-, bis- or tris-(2-hydroxy-lower alkyl)-amines, such as ethanol-, diethanol- or triethanol-amine, tris(hydroxymethyl)methylamine or 2-hydroxy-tertbutylamine, N,N-di-lower alkyl-N-(hydroxy-lower alkyl)-amines, such as N,N-dimethyl-N-(2-hydroxyethyl)-amine, or N-methyl-D-glucamine, or quaternary ammonium hydroxides, such as tetrabutylammonium hydroxide. The compounds of formula I having a basic group, for example an amino group, can form acid addition salts, for example with suitable inorganic acids, for example hydrohalic acids, such as hydrochloric acid or hydrobromic acid, or sulfuric acid with replacement of one or both protons, phosphoric acid with replacement of one or more protons, e.g. orthophosphoric acid or metaphosphoric acid, or pyrophosphoric acid with replacement of one or more protons, or with organic carboxylic, sulfonic, sulfo or phosphonic acids or N-substituted sulfamic acids, for example acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid, as well as with amino acids, such as the .alpha.-amino acids mentioned hereinbefore, and with methanesulfonic acid, ethanesulfonic acid, 2-hydroxyethanesulfonic acid, ethane-1,2-disulfonic acid, benzenesulfonic acid, 4-methylbenzenenesulfonic acid, naphthalene-2-sulfonic acid, 2- or 3-phosphoglycerate, glucose-6-phosphate, or N-cyclohexylsulfamic acid (forming cyclamates) or with other acidic organic compounds, such as ascorbic acid. Compounds of formula I having acid and basic groups can also form internal salts.

For isolation and purification purposes it is also possible to use pharmaceutically unacceptable salts.

Synthesis of Compounds

The compounds of formula (I) can be prepared by known methods for the synthesis of substituted amines. For example, a compound of the formula

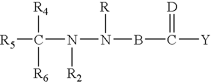

may be prepared by reaction of an amine of the formula

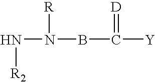

with a substituted alkyl halide of the formula

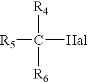

Compounds of formula (IA) may be prepared by reacting an amine of formula

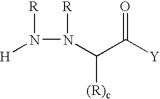

with a halide of formula

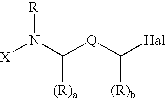

Compounds of formula (IB) may be prepared by reacting an amine of formula

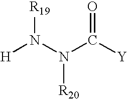

with a halide of formula

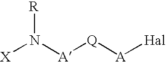

The compounds of formula (IC) can be prepared by reacting a compound of formula (II)

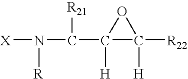

wherein X, R₂₁, R₂₂ and R have the significance given earlier, with a compound of formula (III)

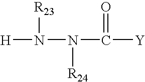

wherein R₂₃, R₂₄ and Y have the significance given earlier.

A compound of formula (ID) may be obtained from a compound of formula (IC) by oxidation in accordance with known methods of oxidative transformations of alcohols to ketones.

A compound of formula (ID) may be also be obtained by reacting a compound of formula (IIa)

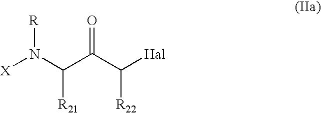

wherein X, R, R₂₁ and R₂₂ are as previously defined and Hal is a group selected from —Cl, —Br, —I or —OS(O)₂R, with a compound of formula (III).

The methods of preparation of compounds of formula (IC) and (ID) may be represented by the following general Schemes 1 to 3.

In the Schemes presented herein, the following abbreviations are made:

AA refers to amino acid or amino acid residue; AcCN refers to acetonitrile; BOP refers to benzotriazol-1-yloxytris(dimethylamino)-phosphonium hexafluorophosphate; CBZ refers to carbobenzoxy; CDI refers to N,N′-carbonyldiimidazole; DMF refers to dimethylformamide; DMSO refers to dimethylsulfoxide; HBT refers to 1-hydroxybenzotriazole; Py refers to pyridine; PyxSO₃ refers to the pyridine complex of sulfur trioxide; RT refers to room temperature and L-Val refers to L-valine.

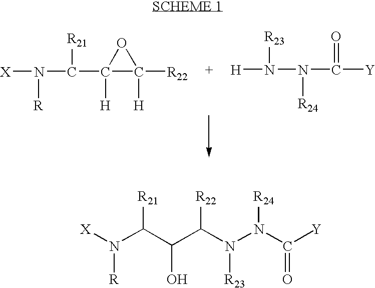

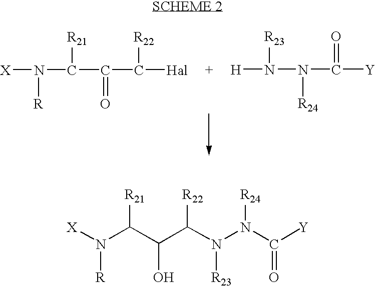

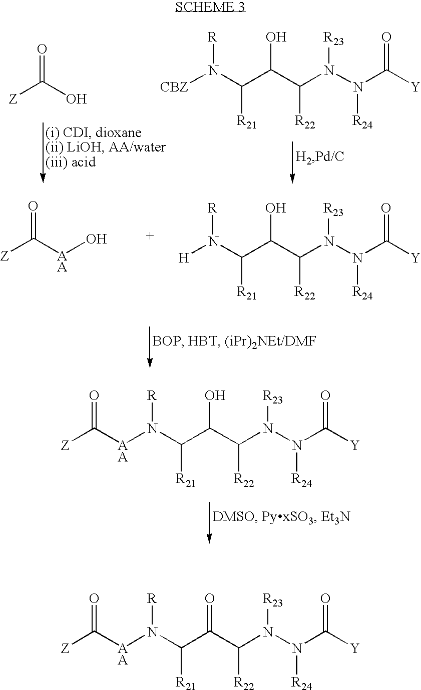

The reaction schemes illustrated can be carried out by generally known methods as exemplified hereinafter. The amino acids or peptide mimics for use in the synthesis of compounds of this invention are generally commercially available or may De prepared by conventional methods of organic chemistry.

Synthetic routes to the intermediates (II), (IIa) and (III) are readily available. The chiral aminoalkylepoxides of formula (II) can be obtained using methods described in the following:

(a) Evans, B. E., et al., J. Org. Chem., 50, 4615-4625 (1985)

(b) Luly, J. R., et al., J. Org. Chem., 52, 1487-1492 (1987);

(c) Handa, B. K., et al., European Patent Application No. 346,847-A2 (1989); and (d) Marshall, G. R., et al., International Patent Application No WO91/08221.

The N-protected aminoalkyl halomethylketones (IIa) are commercially available or can be prepared using methods described in: (e) Rich, et al., J. Med. Chem., 33, 1285-1288 (1990) and reference (d) above.

The hydrazide intermediates (III) can be obtained using known methods such as those described in the following: (g) Dutta, A. S., et al, J. Chem. Soc. Perkin Trans. I, (1975) 1712-1720; (h) Ghali, N. I., et al., J. Org. Chem., 46, 5413-5414 (1981), (i) Gante, J., Synthesis, (1989) 405-413 and (j) Houben-Weyl's Methoden der Organische Chemie, vol. 16a, Part 1, pp 421-855; Georg Thieme Verlag, Stuttgart (1990).

The present invention may be better understood with reference to the following examples. These examples are intended to be representative of specific embodiments of the invention, and are not intended as limiting the scope of the invention.

### EXAMPLES

### Example A

Enzyme Inhibition Assay

The compounds of the invention are analyzed for inhibitory activity by use of the MBP-C125 assay. This assay determines the relative inhibition of beta-secretase cleavage of a model APP substrate, MBP-C125SW, by the compounds assayed as compared with an untreated control. A detailed description of the assay parameters can be found, for example, in U.S. Pat. No. 5,942,400. Briefly, the substrate is a fusion peptide formed of maltose binding protein (MBP) and the carboxy terminal 125 amino acids of APP-SW, the Swedish mutation. The beta-secretase enzyme is derived from human brain tissue as described in Sinha et al, 1999, Nature 40:537-540) or recombinantly produced as the full-length enzyme (amino acids 1-501), and can be prepared, for example, from 293 cells expressing the recombinant cDNA, as described in WO00/47618.

Inhibition of the enzyme is analyzed, for example, by immunoassay of the enzyme's cleavage products. One exemplary ELISA uses an anti-MBP capture antibody that is deposited on precoated and blocked 96-well high binding plates, followed by incubation with diluted enzyme reaction supernatant, incubation with a specific reporter antibody, for example, biotinylated anti—SW192 reporter antibody, and further incubation with streptavidin/alkaline phosphatase. In the assay, cleavage of the intact MBP—C125SW fusion protein results in the generation of a truncated amino-terminal fragment, exposing a new SW-192 antibody-positive epitope at the carboxy terminus. Detection is effected by a fluorescent substrate signal on cleavage by the phosphatase. ELISA only detects cleavage following Leu 596 at the substrate's APP-SW 751 mutation site.

Specific Assay Procedure:

Compounds are diluted in a 1:1 dilution series to a six-point concentration curve (two wells per concentration) in one 96-plate row per compound tested. Each of the test compounds is prepared in DMSO to make up a 10 millimolar stock solution. The stock solution is serially diluted in DMSO to obtain a final compound concentration of 200 micromolar at the high point of a 6-point dilution curve. Ten (10) microliters of each dilution is added to each of two wells on row C of a corresponding V-bottom plate to which 190 microliters of 52 millimolar NaOAc, 7.9% DMSO, pH 4.5 are pre-added. The NaOAc diluted compound plate is spun down to pellet precipitant and 20 microliters/well is transferred to a corresponding flat-bottom plate to which 30 microliters of ice-cold enzyme-substrate mixture (2.5 microliters MBP—C125SW substrate, 0.03 microliters enzyme and 24.5 microliters ice cold 0.09% TX100 per 30 microliters) is added. The final reaction mixture of 200 micromolar compound at he highest curve point is in 5% DMSO, 20 millimolar NaCAc, 0.06% TX100, at pH 4.5.

Warming the plates to 37 degrees C. starts the enzyme reaction. After 90 minutes at 37 degrees C, 200 microliters/well cold specimen diluent is added to stop the reaction and 20 microliters/well was transferred to a corresponding anti-MBP antibody coated ELISA plate for capture, containing 80 microliters/well specimen diluent. This reaction is incubated overnight at 4 degrees C. and the ELISA is developed the next day after a 2 hour incubation with anti-192SW antibody, followed by Streptavidin-AP conjugate and fluorescent substrate. The signal is read on a fluorescent plate reader.

Relative compound inhibition potency is determined by calculating the concentration of compound that showed a fifty percent reduction in detected signal (IC50) compared to the enzyme reaction signal in the control wells with no added compound.

### Example B

Cell Free Inhibition Assay Utilizing a Synthetic APP Substrate

A synthetic APP substrate that can be cleaved by beta-secretase and having N-terminal biotin and made fluorescent bay the covalent attachment of Oregon green at the Cys residue is used to assay beta-secretase activity in the presence or absence of the inhibitory compounds of the invention. Useful substrates include the following:

| | |
|---|---|
| Biotin-SEVNLDAEFRC [Oregon green] KK | [SEQ ID NO:1] |
| Biotin-SEVKMDAEFRC [Oregon green] KK | [SEQ ID NO:2] |
| Biotin-GLNIKTEEISEISYEVEFRC [Oregon green] KK | [SEQ ID NO:3] |
| Biotin-ADRGLTTRPGSGLTNIKTEEISEVNLDAEFC [Oregon green] KK | [SEQ ID NO:4] |
| Biotin-FVNQHLCₒₓGSHLVEALY-LVCₒₓGERGFFYTPKAC [Oregon green] KK | [SEQ ID NO:5] |

The enzyme (0.1 nanomolar) and test compounds (0.001-100 micromolar) are incubated in pre-blocked, low affinity, black plates (384 well) at 37 degrees for 30 minutes. The reaction is initiated by addition of 150 millimolar substrate to a final volume of 30 microliter per well. The final assay conditions are: 0.001-100 micromolar compound inhibitor; 0.1 molar sodium acetate (pH 4.5); 150 nanomolar substrate; 0.1 nanomolar soluble beta-secretase; 0.001% Tween 20, and 2% DMSO. The assay mixture is incubated for 3 hours at 37 degrees C., and the reaction is terminated by the addition of a saturating concentration of immunopure streptavidin. After incubation with streptavidin at room temperature for 15 minutes, fluorescence polarization is measured, for example, using a LJL Acqurest (Ex485 nm/Em530 nm). The activity of the beta-secretase enzyme is detected by changes in the fluorescence polarization that occur when the substrate is cleaved by the enzyme. Incubation in the presence or absence of compound inhibitor demonstrates specific inhibition of beta-secretase enzymatic cleavage of its synthetic APP substrate.

### Example C

Beta-Secretase Inhibition: P26-P4′SW Assay

Synthetic substrates containing the beta-secretase cleavage site of APP are used to assay beta-secretase activity, using the methods described, for example, in published PCT application WO00/47618.

| | |
|---|---|
| The P26-P4′SW substrate is a pep- | |
| tide of the sequence: | |
| (biotin) | |
| CGGADRGLTTRPGSGLTNIKTEEISEVNLDAIEF | [SEQ ID NO:6] |
| The P26-P1 standard has the sequence: | |
| (biotin) | |
| CGGADRGLTTRPGSGLTNIKTEEISEVNL. | [SEQ ID NO:7] |

Briefly, the biotin-coupled synthetic substrates are incubated at a concentration of from about 0 to about 200 micromolar in this assay. When testing inhibitory compounds, a substrate concentration of about 1.0 micromolar is preferred. Test compounds diluted in DMSO are added to the reaction mixture, with a final DMSO concentration of 5%. Controls also contain a final DMSO concentration of 5%. The concentration of beta secretase enzyme in the reaction is varied, to give product concentrations with the linear range of the ELISA assay, about 125 to 2000 picomolar, after dilution.

The reaction mixture also includes 20 millimolar sodium acetate, pH 4.5, 0.06% Triton X00, and is incubated at 37 degrees C. for about 1 to 3 hours. Samples are then diluted in assay buffer (for example, 145.4 nanomolar sodium chloride, 9.51 millimolar sodium phosphate, 7.7 millimolar sodium azide, 0.05% Triton X405, 6 g/liter bovine serum albumin, pH 7.4) to quench the reaction, then diluted further for immunoassay of the cleavage products.

Cleavage products can be assayed by ELISA. Diluted samples and standards are incubated in assay plates coated with capture antibody, for example, SW192, for about 24 hours at 4 degrees C. After washing in TTBS buffer (150 millimolar sodium chloride, 25 millimolar Tris, 0.05% Tween 20, pH 7.5), the samples Mre incubated with streptavidin-AP according to the manufacturer's instructions. After a one hour incubation at room temperature, the samples are washed in TTBS and incubated with fluorescent substrate solution A (31.2 g/liter 2-amino-2-methyl-1-propanol, 30 mg/liter, pH 9.5). Reaction with streptavidin-alkaline phosphate permits detection by fluorescence. Compounds that are effective inhibitors of beta-secretase activity demonstrate reduced cleavage of the substrate as compared to a control.

### Example D

Assays using Synthetic Oligopeptide-Substrates

Synthetic oligopeptides are prepared that incorporate the known cleavage site of beta-secretase, and optionally detectable tags, such as fluorescent or chromogenic moieties. Examples of such peptides, as well as their production and detection methods are described in U.S. Pat. No: 5,942,400, herein incorporated by reference. Cleavage products can be detected using high performance liquid chromatography, or fluorescent or chromogenic detection methods appropriate to the peptide to be detected, according to methods well known in the art.

By way of example, one such peptide has the sequence (biotin)-SEVNLDAEF [SEQ ID NO: 81, and the cleavage site is between residues 5 and 6. Another preferred substrate has the sequence ADRGLTTRPGSGLTNIKTEEISEVNLDAEF [SEQ ID NO: 9], and the cleavage site is between residues 26 and 27.

These synthetic APP substrates are incubated in the presence of beta-secretase under conditions sufficient to result in beta-secretase mediated cleavage of the substrate. Comparison of the cleavage results in the presence of the compound inhibitor to control results provides a measure of the compound's inhibitory activity.

### Example E

Inhibition of Beta-Secretase Activity—Cellular Assay

An exemplary assay for the analysis of inhibition of beta-secretase activity utilizes the human embryonic kidney cell line HEKp293 (ATCC Accession No. CRL-1573) transfected with APP751 containing the naturally occurring double mutation Lys651Met52 to Asn651Leu652 (numbered for APP751), commonly called the Swedish mutation and shown to overproduce A beta (Citron et al., 1992, Nature 360:672-674), as described in U.S. Pat. No. 5,604,102.

The cells are incubated in the presence/absence of the inhibitory compound (diluted in DMSO) at the desired concentration, generally up to 10 micrograms/ml. At the end of the treatment period, conditioned media is analyzed for beta-secretase activity, for example, by analysis of cleavage fragments. A beta can be analyzed by immunoassay, using specific detection antibodies. The enzymatic activity is measured in the presence and absence of the compound inhibitors to demonstrate specific inhibition of beta-secretase mediated cleavage of APP substrate.

### Example F

Inhibition of Beta-Secretase in Animal Models of AD

Various animal models can be used to screen for inhibition of beta-secretase activity. Examples of animal models useful in the invention include, but are not limited to, mouse, guinea pig, dog, and the like. The animals used can be wild type, transgenic, or knockout models. In addition, mammalian models can express mutations in APP, such as APP695-SW and the like described herein. Examples of transgenic non-human mammalian models are described in U.S. Pat. Nos. 5,604,102, 5,912,410 and 5,811,633.

PDAPP mice, prepared as described in Games et al., 1995, Nature 373:523-527 are useful to analyze in vivo suppression of A beta release in the presence of putative inhibitory compounds. As described in U.S. Pat. No. 6,191,166, 4 month old PDAPP mice are administered compound formulated in vehicle, such as corn oil. The mice are dosed with compound (1-30 mg/ml; preferably 1-10 mg/ml). After time, e.g., 3-10 hours, the animals are sacrificed, and brains removed for analysis.

Transgenic animals are administered an amount of the compound inhibitor formulated in a carrier suitable for the chosen mode of administration. Control animals are untreated, treated with vehicle, or treated with an inactive compound. Administration can be acute, i.e., single dose or multiple doses in one day, or can be chronic, i.e., dosing is repeated daily for a period of days. Beginning at time 0, brain tissue or cerebral fluid is obtained from selected animals and analyzed for the presence of APP cleavage peptides, including A beta, for example, by immunoassay using specific antibodies for A beta detection. At the end of the test period, animals are sacrificed and brain tissue or cerebral fluid is analyzed for the presence of A beta and/or beta-amyloid plaques. The tissue is also analyzed for necrosis.

Animals administered the compound inhibitors of the invention are expected to demonstrate reduced A beta in brain tissues or cerebral fluids and reduced beta amyloid plaques in brain tissue, as compared with-non-treated controls.

### Example G

Inhibition of A Beta Production in Human Subjects

Subjects suffering from Alzheimer's Disease (AD) demonstrate an increased amount of A beta in the brain. AD subjects and patients are administered an amount of the compound inhibitor formulated in a carrier suitable for the chosen mode of administration. Administration is repeated daily or the duration of the test period. Beginning on day 0, cognitive and memory tests are performed, for example, once per month.

Subjects administered the compound inhibitors are expected to demonstrate slowing or stabilization of disease progression as analyzed by changes in one or more of the following disease parameters: A beta present in CSF or plasma; brain or hippocampal volume; A beta deposits in the brain; amyloid plaque in the brain; and scores for cognitive and memory function, as compared with control, non-treated subjects.

### Example H

Prevention of A Beta Production in Subjects at Risk for AD

Subjects predisposed or at risk for developing AD are identified either by recognition of a familial inheritance pattern, for example, presence of the Swedish Mutation, and/or by monitoring diagnostic parameters. Subjects identified as predisposed or at risk for developing AD are administered an amount of the compound inhibitor formulated in a carrier suitable for the chosen mode of administration. Administration is repeated daily for the duration of the test period. Beginning on day 0, cognitive and memory tests are performed, for example, once per month.

Subjects administered the compound inhibitors are expected to demonstrate slowing or stabilization of disease progression as analyzed by changes in one or more of the following disease parameters: A beta present in CSF or plasma; brain or hippocampal volume; amyloid plaque in the brain; and scores for cognitive and memory function, as compared with control, non-treated subjects.

All temperatures are in degrees Celsius.

Examples of compounds of formula (I) include those pounds of formula (IV) presented in Table 1:

TABLE 1

| | |
|---|---|
| | (IV) |
| 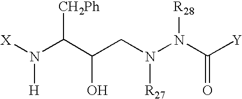 | |

| | Ex. | | | | |
|---|---|---|---|---|---|
| No. | No | X | R₂₇ | R₂₈ | Y |
| 1 | (8) | CBZ- | 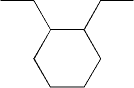 | | t-BuO- |
| 2a | (10) | QC-Asn- | 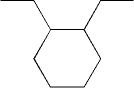 | | t-BuO- |
| 2b | (23) | QC-Asn-- | 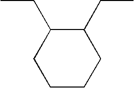 | | t-BuO- |
| 2b.A. | (23A) | QC-Asn- | 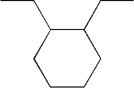 | | t-BuO- |
| 3 | (9) | QC-Val- | 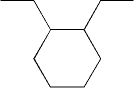 | | t-BuO- |
| 4 | (12) | QC-Gln- | 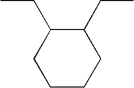 | | t-BuO- |
| 5 | (13) | QC-Thr- | 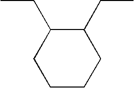 | | t-BuO- |
| 6 | (11) | QC-Val- | 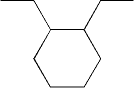 | | t-BuO- |
| 7A | (3) | QC-Asn- | i-Pr- | H | t-BuO- |
| 7B | (20) | QC-Asn- | i-Pr- | H | t-BuO- |
| 8 | (4) | QC-Asn- | i-Pr- | H | (2-PCNH)Ph- |
| 9 | (2) | QC-Val- | i-Pr- | H | t-BuO- |
| 10 | (16) | PC-Val- | 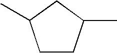 | | t-BuO- |
| 11 | (18) | QC-Asn- | 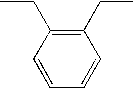 | | t-BuO- |
| 12 | (7) | QC-Asn- | 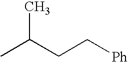 | H | t-BuO- |
| 13 | (25) | QC-Asn- | i-Pr- | H | t-Bu- |
| 14 | (26) | QC-Asn- | i-Pr- | H | t-BuNH- |
| 15 | (27) | PIC-Asn- | i-Pr- | H | t-BuO- |
| 16 | (30) | QC-Asn- | Bzl- | H | t-BuO- |
| 17 | (32) | QC-Asn- | cyclohexyl | H | t-BuO- |
| 18 | (35) | BZ-Asn- | i-Pr- | H | t-BuO- |

| | | | | |
|---|---|---|---|---|
| 19 | (37) | QC-Asn- | -(CH₂)₄- | t-BuO- |
| 20 | (38) | QC-CNAla- | 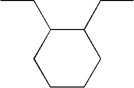 | t-BuO- |

In the above Table, CBZ refers to benzyloxycarbonyl; QC refers to quinoline-2-carbonyl; PC refers to 2-pyridinemethoxycarbonyl; Asn refers to asparagine; Val refers to valine; Gln refers to glutamine and Thr refers to threonine, BZ refers to benzoyl, PIC refers to picolinyl and CNAla refers to 3cyano-L-alanine.

In the following examples, melting points were taken on a hot stage apparatus and are uncorrected. Proton NMR spectra were recorded at 100 MHz or 300 MHz on Perkin Elmer R32 or Bruker EM 300 spectrometers, respectively. Chemical shifts are ppm downfield from tetramethylsilane. Molecular weights of the compounds presented in Examples 1 to 23 were confirmed by electrospray mass spectrometry analysis, performed in the Department of Chemistry at La Trobe University, Melbourne. Thin layer chromotography (TLC) was performed on silica gel 60-F254 plates (Merck). Compounds were visualized by ultraviolet light and/or 2% aqueous potassium permanganate solution. The compositions (by volume) of the TLC solvent system were as follows: (A)=hexane/ethyl acetate 4:1; (B)=hexane/ethyl acetate 3:2; (C)=ethyl acetate; (D)=chloroform/methanol 23:2.

### Example 1

### t-Butyl 3-isopropyl-[(2R,3S)-2-hydroxy-3-(phenylmethoxycarbonyl)amino-4-phenylbutyl)carbazate

Step A: t-Butyl 3-isopropyl carbazate: The title compound can be prepared by method of Dutta et al., J.C.S. Perkin I, 1975, 1712-1720 or by the following procedure: A mixture of 13.2 g (0.1 mol) of t-butyl carbazate and 6 g (0.103 mol) of acetone and 12.5 g (0.1 mol) of anhydrous magnesium sulfate in 100 mL of methylene chloride was stirred for 12 hr. at room temperature. After removal Of the drying agent by filtration the filtrate was evaporated to dryness under reduced pressure to give 16.9 g (98% yield) of corresponding hydrazone melting 104°-105° C. after crystallization from cyclohexane. To a suspension of 2.04 g (0.094 mol) of lithium borohydride in 100 mL of dry THF, 12 mL (0.094 mol) of chlorotrimethylsilane was added under nitrogen at room temperature. After 30 min. of stirring, 13.45 g (0.078 mol) of hydrazone was slowly added at room temperature and stirring was continued for 2 hr. Then 50 mL of methanol was carefully added and the mixture was evaporated to dryness under reduced pressure. The residue was partitioned between ether (150 mL) and water (50 mL). The organic phase was dried over anhydrous magnesium sulfate and filtered off. Dry hydrogen chloride was passed through the filtrate and the white solid formed was removed by filtration, washed with a fresh portion of ether and dried to give 10.5 g of hydrochloride salt of the title compound. This was transformed into a free base by partition between hexane (150 mL) and 20% aqueous potassium hydroxide. Yield 8.3 g (61%).

Step B: t-Butyl 3-1sopropyl-[(2R,3S)-2-hydroxy-3-(phenylmethoxycarbonyl)amino-4-phenylbutyl]carbazate: A mixture of 0.15 g (0.45 mmol) of N-CBZ-L-phenylalanine chloromethyl ketone and 1 mL of a saturated solution of sodium iodide in dry DMF was stirred for 15 min. at room temperature. To this, 0.074 g (0.47 mmol) of t-butyl 3-isopropyl carbazate was added followed by 0.095 g (1.13 mmol) of sodium bicarbonate. After 6 hours of stirring at room temperature, 0.051 g (1.3 mmol) of sodium borohydride was added and stirring was continued for an additional 30 min. The solution was diluted to 30 mL with ethyl acetate and washed with 2% aqueous potassium bisulfate solution, water and saturated aqueous sodium chloride solution, and then dried over anhydrous magnesium sulfate. Evaporation of the solvent under reduced pressure and purification of the residue by flash chromatography (silica gel; hexane/ethyl acetate 20:5) gave the title compound, melting at 118°-119.5° C., in 49% yield; R(A)=0.11; R (B)=0.47; NMR (CDCl₃) 1.0 (m, 6H, isopropyl CH₃); 1.4.4 (s, 9H, t-butyl CH₃) ; 2.62 (m, 2H, butyl CH₂-1); 2.75-3.2 (m, 3H, butyl CH-3, CH2-4; 3.47 (m, 1 H, isopropyl CH); 3.89 (m, 1 H, butyl CH-2); 4.44 (broad s, 1 H, OH); 4.6 (broad m, 1H, NH); 5.03 (s, 2H, methoxy CH₂); 5.3 (broad s, 1H, carbazate NH); 7.23 (m, 10H, aromatic).

### Example 2

### t-Butyl 3-isopropyl-3-[(2R,3S)-2-hydroxy-3-(N-quinaldoyl-L-valyl)amino-4-phenylbutyl]carbazate

Step A: N-Quinaldoyl-L-Valine: A mixture of 0.62 g (3.6 mmol) of quinaldic acid and 0.61 g (3.76 mmol) of 1,1′-carbonyldiimidazole in 1 mL of dry 1,4-dioxane was stirred for 30 rain at room temperature. To this, a solution of 0.43 g (3.7 mmol) of L-valine and 0.155 g (3.7 mmol) of lithium hydroxide in 1 mL of water was added and the resulting mixture was stirred vigorously at room temperature for about 4 hours; The mixture was diluted to 10 mL with water, cooled (ice-water bath), then acidified with 1N hydrochloric acid to pH about 3 and allowed to stand for 2 hours at 4° C. The crystals that formed were removed by filtration, washed three times with 5 mL of cold water and dried under high vacuum over phosphorus pentoxide to give 0.75 g of the product. Yield=76%, melting point 1340-1360C., NMR (DMSO-d₆) 1.03 (d, 6H, val CH₃); 2.3 (m, 1H, val CH-J3); 3.35 (broad s, 1H, OH); 4.49 (q, 1 H, val CH—); 7.5-8.3 (m, 5H, aromatic); 3.5-8.76 (m, 2H, aromatic, NH).

Step B: t-Butyl 3-isopropyl-3-[(2R,3S)-3-amino-2-hydroxy-4-phenylbutyl]carbazate: To a chilled solution of 0.113 g (0.24 mmol) of the product of Example 1 in 2 mL of methanol was added 0.1 g of 10% palladium on activated carbon under nitrogen, followed by 0.1 g of sodium borohydride. The reaction was allowed to warm to room temperature and stir for 1 hour, then catalyst was removed by filtration and washed with fresh portion of methanol. The combined filtrates were treated with 1 mL of 0.1N aqueous solution of hydrochloric acid and evaporated to dryness under reduced pressure. The residue was treated with 5 mL of 0.1N potassium hydroxide and the product was taken up with 30 mL of diethyl ether. The organic phase was washed with saturated aqueous sodium chloride solution, dried over anhydrous magnesium sulfate and evaporated under reduced pressure to give 0.0797 g (99% yield) of the Step B product, which was used in the next step without further purification.

Step C: t-Butyl 3-isopropyl-3-[(2R,3S)-2-hydroxy-3-(N-quinaldoyl-L-valyl)amino-4-phenylbutyl]carbazate: To a mixture of 0.0643 g (0.24 mmol) of the acid from Step A, 0.0797 g (0.236 mmol) of the amine from Step B, 0.032 g (0.24 mmol) of 1-hydroxybenzotriazole in 0.5 mL of anhydrous DMF was added 0.071 g (0.24 mmol) of 1-(3-dimethylaminopropyl)-3-ethylcarbodiimide methyliodide. After stirring overnight at room temperature the mixture was diluted to 30 mL with ethyl acetate and washed successively with water, 5% aqueous sodium bicarbonate, 2% aqueous potassium bisulfate solution, and saturated sodium chloride solution and dried over anhydrous magnesium sulfate. Evaporation of the solvent under reduced pressure and purification of the residue by column chromatography (silica gel, hexane/ethyl acetate 3:2) gave 0.091 g (65% yield) of the title compound, melting at 186°-189° C.: Rf (B)=0.19; Rf (C)=0.83; NMR (CDCl₃) 1.0 (m, 12H, val and isopropyl CH₃) ; 1.71 (s, 9H, t-butyl CH₃); 2.3 (m, 1 H, val CH—); 2.5-3.27 (m, 3H, butyl CH-3, CH₂); 3.5 (m, 1 H, isopropyl CH); 4.31 (m, 2H, val CH—, OH); 5.43 (broad s, 1 H, carbazate NH); 6.22 (broad d, 1H, butyl NH); 6.7-8.73 (m, 12H, aromatic, NH).

### Example 3

### t-Butyl 3-isopropyl-3-[(2R,3S)-2-hydroxy-3-(N-quinaldoyl-L-asparaginyl)amino-4-phenylbutyl]carbazate

Step A: N-Quinaldoyl-L-asparagine: When L-asparagine was substituted for L-valine in Step A of Example 2, the identical process afforded the title compound, melting at 200°-203° C., in 85% yield, NMR (DMSO-d.sub.6) 3.0 (m, 2H, asn CH₂); 5.0 (m, 1H, asn CH—); 6.3 (broad s, 1H, OH); 6.55 (broad s, 1H, NH₂); 7.3 (broad s, 1 H, NH₂); 7.55-8.6 (m, 6H, aromatic); 9.22 (d, 1 H, NH).

Step B: t-Butyl 3-isopropyl-3-[(2R,3S)-2-hydroxy-3-(N-quinaldoyl-L-asparaginyl)amino-4-phenylbutyl]carbazate: To a stirred solution of the product of Step A (0.111 g; 0.386 mmol), the product of Example 2, Step B (0.13022 g; 0.386 mmol), benzotriazol-1-yloxytris dimethylamino)phosphonium hexafluorophosphate (0.205 g; 0.46 mmol) and 1-hydroxybenzotriazole (0.052 g; 0.384 mmol) in 1 mL of anhydrous DMF was added, N,N-diisopropylethylamine (0.24 ml; 1.38 mmol). After stirring for 12 hours at room temperature the reaction was diluted to 30 mL with ethyl acetate and washed with water, 2% potassium bisulfate, 5% sodium bicarbonate and saturated aqueous sodium chloride solution and dried over anhydrous magnesium sulfate. Evaporation of the solvent under reduced pressure and purification of the residue by column chromatography (silica gel, ethyl acetate) gave 0.152 g (65% yield) of the title product melting at 109°-114° C.; Rf (C)=0.36; Rf (D)=0.37; NMR (CDCl₃) 1.0 (m, 6H, val, isopropyl CH₃); 1.42 (s, 9H, t-butyl CH₃); 2.5-3.1 (m, 7H, asn CH₂, butyl CH₂-1,-4, CH-3); 3.44 (m, 1 H, isopropyl CH); 4.21 (m, 1 H, butyl CH-2); 4.55 (s, 1 H, OH); 4.94 (m, 1H, asn CH—); 5.4-6.2 (m, 3H, amide); 6.7-8.4 (m, 11H, aromatic); 9.25 (m, 1H, NH).

### Example 4

### 1-(2-pyridyl)methoxycarbonylanthraniloyl-2-[(2R,3S)-2-hydroxy-3-(N-quinaldo yl-L-asparaginyl)amino-4-phenylbutyl]-2-isopropyl-hydrazine

Step A: (2-Pyridyl)methoxycarbonylanthranilic acid: Phosgene was bubbled through a solution of 10 g (66 mmol) of methylanthranilate in 15 mL of anhydrous toluene for 2 hours at reflux. Then the solvent was distilled off under reduced pressure to give 11.7 g (100%) of 2-methoxycarbonylphenylisocyanate; NMR (CDCl₃) 3.89 (s, 3H, CH₃); 7.0-7.63 (m, 3H, phenyl H-3,-4,-5); 8.0 (dd, 1 H, phenyl H-6). This was converted to the title compound, in 34% overall yield, by condensation with an equimolar amount of 2-pyridylcarbinol followed by saponification of the resulting ester with 1N sodium hydroxide and acidification of the reaction mixture to pH 4. The crude product was purified by crystallization from ethyl acetate; melting point=172°-175° C.; NMR (DMSO-d₆) 5.2 (s, 2H, methoxy CH₂); 6.8-8.8 (m, 9H, aromatic, NH); 10.8 (broad s, 1H, OH).

Step B: 2-((2R,3S)-2-hydroxy-3-(N-quinaldoyl-L-asparaginyl)amino-4-phenylbutyl]-2-isopropyl-hydrazine: Hydrogen chloride gas was bubbled through the solution of 0.1 g (0.165 mmol) of product of Example 3 in 10 mL of 1% solution of methanol in methylene chloride for 30 min at room temperature. After washing the excess of HCl with nitrogen the solvent was removed under reduced pressure to give 0.089g (100%) of the title compound as a white solid.

Step C: 1-(2-pyridyl)methoxycarbonylanthraniloyl-2-[(2R,3S)-2-hydroxy-3-N-quinaldoyl-L-asparaginyl)amino-4-phenylbutyl]-2-isopropyl-hydrazine: Coupling the products of Step A and B, using the general procedure outlined in Example 3; Step B, gave the title compound in 24% yield, after purification by column chromatography (silica gel, ethyl acetate); melting point=96°-112° C.; Rf (C)=0.13; Rf (D)=0.36; NMR (CDCl₃) 1.18 (m, 6H, isopropyl CH₃); 1.8-3.4 (m, 8H, asn CH₂, butyl CH₂−1, −4, CH-3; OH); 3.6 (m, 1H, isopropyl CH); 4.2 (m, 1H, butyl CH-3); 4.5-5.18 (m, 2H, asn CH—, hydrazide NH); 5.35 (s, 2H, methoxy CH₂); 5.3-6.5 (broad m, 2H, asn NH₂); 6.8-8.8 (m, 20H, aromatic, butyl NH); 9.14 (m, 1H, asn NH); 10.36 (s, 1H, anthr. NH).

### Example 5

### t-Butyl 3-isopropyl-3-[(2-oxo-3(S)-(N-quinaldoyl-L-asparaginyl)amino-4-phenylbutyl]carbazate

To a mixture of 0.0533 g (0.088 mmol) of the product of Example 3 and 0.049 g (0.31 mmol) of sulfur trioxide pyridine complex in 1 mL of anhydrous DMSO 0.043 mL (0.31 mmol) of triethylamine was added. After stirring for 45 rain at room temperature the reaction mixture was poured on ice and allowed to warm to room temperature. The precipitate which formed was removed by filtration, washed with water and dried overnight in vacuo to give 0.044 g (83% yield) of the title compound which was further purified by crystallization from the aqueous methanol; melting point=146°-150° C.; Rf (D)=0.32; NMR (CDCl₃) 1.0 (d, 6H, isopropyl CH₃); 1.38 (s, 9H, t-butyl CH₃);-2.5-3.3 (m, 5H, asn CH₂, butyl CH₂, isopropyl CH); 3.7 (s, 2H, butyl. CH₂); 4.6-5.3 (m, 2H, asn CH, butyl CH-3); 5.6 (broad s, 1 H, NH); 6.09 (broad m, 2H, 2x NH); 6.9-8.4 (m, 12 H, aromatic, N); 9.2 (broad d, 1H, asn NH).

### Example 6

### t-Butyl 3-(1-methyl-3-phenylpropen-3-yl)-3-[(2R and S, 3S)-2-hydroxy-3-(phenylmethoxycarbonyl)amino-4-phenylbutyl]carbazate

Step A: 2R, S)-3(S)-1,2-Epoxy-3-phenylmethoxycarbonylamino-4-phenylbutane: To the solution of 6 g (18 mmol) of N-CBZ-L-phenylalanine chloromethyl ketone in 30 mL of 50% methanolic tetrahydrofuran was added 0.68 g of sodium borohydride. After stirring for 30 min at room temperature the mixture was carefully acidified with 1N hydrochloric acid and evaporated to dryness under reduced pressure. The residue was diluted to 50 mL with methylene chloride, washed with water and saturated aqueous sodium chloride and dried over anhydrous magnesium sulfate. Evaporation gave 6.02 g (100%) of 2(R,S)-3(S)-1-chloro-2-hydroxy-3-phenylmethoxycarbonylamino-4-phenylbutane, as a white solid. This was dissolved in 50 mL of isopropanol and 9 mL of 2N methanolic potassium hydroxide was added at room temperature. After stirring for 1 hour at room temperature the solvent was removed under reduced pressure and the residue was partitioned between 50 mL of ethyl acetate and 20 mL of water. The organic phase was washed with saturated aqueous sodium chloride, dried over anhydrous magnesium sulfate and evaporated to dryness to give 5.3 g (99% yield) of the title compound as the predominantly 2(S) stereoisomer as determined from relative integration of erythro-NCH (3.74 ppm; 72%) and threo-NCH (4.2; 28%); NMR (CDCl₃) 2.42-3.17 (m, 5H, butane CH₂ −1, −4, CH-2); 3.74 (m, 0.72H, butane CH-3); 4.2 (m, 0.28H, butane CH-3); 4.73 (broad m, 1H, NH); 5.08 (s, 2H, methoxy CH₂); 7.3 (m, 10H, aromatic).

Step B: t-Butyl 3-(1-methyl-3-phenylpropen-2-yl)carbazate: This compound was prepared by the method of Ghali et al. (J. Org. Chem., 1981, 46, 5413-5414) in about 65% overall yield, from trans-4-phenyl-3-buten-2-one and t-butyl carbazate, after crystallization of the crude product from hexane; melting point=76°-79° C.; NMR (CDCl₃) 1.24 (d, 3H, CH₃); 1.45 (s, 9H, t-butyl CH₃); 3.78 (m, 2H, propenyl CH-1, carbazate NH-3); 5.8-6.29 (m, 2H, carbazate NH-2, propenyl CH-2); 6.53 (d, 1 H, propenyl CH-3); 7.3 (m, 5H, aromatic).

Step C: t-Butyl 3-(1-methyl-3-phenylpropen-3-yl)-3-[(2R and S, 3S)-2-hydroxy-3-(phenylmethoxycarbonyl)amino-4-phenylbutyl]carbazates: 0.57 g of epoxide from Step A in about 15 mL of anhydrous ether was added at room temperature to a vigorously stirred suspension of 8 g of alumina (E. Merck I) impregnated with 1 g (3.81 mmol) of the product of Step B. The stirring was continued for 16 hours and the catalyst was removed by filtration and washed with ethyl acetate (3×25 ml). The combined filtrates were evaporated to dryness under reduced pressure and the residue was separated and purified by column chromatography (silica gel, hexane/ethyl acetate 4:1). The product fractions were evaporated in vacuo to give the 2R, 3S isomer (0.298 g; 28%) and the 2S, 3S isomer (0.1 g; 9%) of the title compound as a white solid.

Isomer 2R, 3S: melting point=101°-104° C.; R_{f} (A)=0.19; NMR (CDCl₃) 1.27 (dd, 3H, CH₃); 1.42 (s, 9H, t-butyl CH₃); 2.67 (m, 2H, butyl CH₂ −1); 3.0 (m, 2H, butyl CH₂ −4); 3.5 (m, 2H, propenyl CH-1, butyl CH-3); 3.91 (m, 1H, butyl CH-2); 4.4, 4.82, 5.38 (broad multiplets, 3×H, amide NH, OH); 5.0 (s, 2H, methoxy CH₂) 6.09 (dd, 1H, propenyl CH-2); 6.5 (d, 1H, propenyl CH-3); 7.22 (m, 15H, aromatic).

Isomer 2S, 3S: melting point=128°-130° C.; R_{f} (A)=0.26; NMR (CDCl₃) 1.22(m, 3H, CH₃); 1.4 (s, 9H, t-butyl CH₃); 2.55 (broad m, 2H, butyl CH₂ −1); 2.95 (d, 2H, butyl CH₂ −4); 3.5 (m, 3H, propenyl CH-2, butyl CH-2, −3); 4.44 (m, 1 H, OH); 5.05 (m, 2H, methoxy CH₂); 5.34 (m, 2H, NH); 6.08 (dd, 1H, propenyl CH-2); 6.5 (d, 1 H, propenyl CH-3); 7.3 (m, 15H, aromatic).

### Example 7

### t-Butyl 3(1-methyl-3-phenylpropyl)-3-[(2R,3S)-2-hydroxy-3-(N-quinaldoyl-L-asparaginyl)amino-4-phenylbutyl]carbazate

Step A: t-Butyl 3-(1-methyl-3-phenylpropyl)-3-[(2R,3S)-2-hydroxy-3-amino-4-phenylbutyl]carbazate: This was prepared in 98% yield by hydrogenolysis of the isomer 2R,3S of the product of Example 6, Step C, performed as described in Example 2, Step B, as white solid.

Step B: t-Butyl 3-(1-methyl-3-phenylpropyl)-3-[(2R,3S)-2-hydroxy-3-(N-quinaldoyl-L-asparaginyl)amino-4-phenylbutyl]-carbazate: The condensation of the amine from Step A (0.0835 g; 0.195 mmol) with N-quinaldoyl-L-asparagine (Example 3, Step A)-(0.0563 g; 0.196 mmol), under condition given in Step B of Example 3, gave 0.11 g (81% yield) of the title compound after purification by column chromatography (silica gel, chloroform/methanol 23:2); melting point=141°-143° C.; R_{f} (C)=0.53, R_{f} (D)=0.38; NMR (CDCl₃) 0.7-2.1 (m, 15H, CH₃, t-butyl CH₃, propyl CH₂ −2, OH); 2.4-3.26 (m, 8H, butyl CH₂ −1, −4, asn CH₂, propyl CH₂ −3); 3.5 (m, 1H, propyl CH-1); 4.22 (m, 1H, butyl CH-3); 4.7 (m, 1H, carbazate NH); 4.95 (m, 1 H, asn CH—); 5.24-6.4 (m, 3H, NH₂, NH); 6.5-8.5 (m, 16H, aromatic); 9.14 (d, 1H, asn NH).

### Example 8

### cis-1,6-3-t-Butoxycarbonyl-4-[(2RS,3S)-2-hydroxy-3-(phenylmethoxycarbonyl)amino-4-phenylbutyl]-3,4-diaza-bicyclo-[4,4,0]decane

Step A: cis-1,6-3-t-Butoxycarbonyl-3,4-diaza-bicyclo[4.4.0]-decane: Cis-1,2-cyclohexanedimethanol was conveyed quantitatively to cis-1,2-cyclohexanedimethyliodide by the general method (Vogel's Textbook of Practical Organic Chemistry, 4th Ed. p. 393, Longman Group Limited, London 1978). An alkylation of 1-benzyloxycarbonyl-2-t-butoxycarbonylhydrazine (Dutta et al., J.C.S. Perkin I, 1975, 1712-1720) with cis-1,2-cyclohexanedimethyliodide, in the presence of two equivalents of Sodium hydride by the method of Dutta et al (J.C.S. Perkin I, 1975, 1712-1720) gave cis-1,6-4-benzyloxycarbonyl-3-t-butoxycarbonyl-3,4-diazabicyclo[4.4.0]-decane in 24% yield, after purification on column chromatography (silica gel, hexane); melting point=68°-69.5° C.; NMR (CDCl₃) 1.0-2.2 (m, 19H, CH₂ −7,8,9,10, CH-1,6); 3.15 (m, 2H, CH₂ −5); 3.82 (m, 2H, CH₂ −2); 5.11 (m, 2H, benzyl CH₂); 7.3 (s, 5H, aromatic). This was converted to the title compound in 95% yield by hydrogenolysis, performed as described in Example 2, Step B; melting point=55°-63° C.; NMR (CDCl₃) 1.0-2.05 (m, 19H, CH₂ −7,8,9,10, CH-1,6); 2.82 (m, 2H, CH₂ −5); 3.33 (m, 2H, CH₂ −2), 4.0 (broad s, 1H, NH).

Step B: cis-1,6-3-t-Butoxycarbonyl-4-[(2RS,3S)-2-hydroxy-3-(phenylmethoxycarbonyl)amino-4-phenylbutyl]-3,4-diaza-bicyclo-[4.4.0]decane: When the product of Step A was substituted for t-butyl 3-(1-methyl-3-phenylpropen-2-yl)carbazate in Example 6, Step C, the identical process afforded the title compound, melting at 98°-103° C., in 42% yield, after purification on column chromatography (silica gel, hexane/ethyl acetate 4:1); R_{f} (A)=0.2, 0.3; R_{f} (B)=0.55, 0.63; NMR (CDCl₃) 1.0-2.18 (m; 19H, decane CH₂ −7,8,9,10, CH-1,6, t-butoxy CH₃); 2.42 (m, 2H, decane CH₂ −5); 2.78-4.5 (m, 9H, butyl CH₂ −1, 4, CH-2,3, decane CH₂ −2, OH); 4.8 (broad m, 1H, NH); 5.0 (s, 2H, methoxy CH₂); 7.22 (m, 10H, aromatic).

### Example 9

### cis-1,6-3-t-Butoxycarbonyl-4-[(2RS,3S)-2-hydroxy-3-(N-quinaldoyl-L-valyl)amino-4-phenylbutyl]-3,4-diaza-bicyclo[4.4.0]decane

When the product of Example 8 is substituted for t-butyl-3-isopropyl-3-[(2R,3S)-2-hydroxy-3-(phenylmethoxycarbonyl)amino-4-phenylbutyl]carbazate in Example 2, the identical process afforded the title compound in 52% yield, after purification by column chromatography (silica gel, hexane/ethyl acetate 3:2); melting point=95°-101° C.; R_{f} (B)=0.32; R_{f} (C)=0.85; NMR (CDCl₃) 0.64-1.93 (m, 25H, val CH₃, decane CH₂ −7,8,9,10, CH-1,6, t-butoxy CH₃); 2.38 (m, 3H, decane CH₂ −5, val CH—); 2.73-3.82 (m, 7H, decane CH₂ −2, butyl CH₂ −1, 4, CH-3); 3.82-5.35 (m, 3H, val CH—, butyl CH-2, OH); 6.0-9.0 (m, 13H, aromatic, NH).

### Example 10

### cis-1,6-3-t-Butoxycarbonyl-4-[(2RS,3S)-2-hydroxy-3-(N-quinaldoyl-L-asparaginyl)amino-4-phenylbutyl]-3,4-diaza-bicyclo[4.4.0]decane

According to Example 2, Step B, the product of Example 8 was converted quantitatively to cis-1,6-3-t-butoxycarbonyl-4-[(2RS,3S)-2-hydroxy-3-amino-4-phenylbutyl]-3,4-diaza-bicyclo[4.4.0]decane. This material was coupled with N-quinaldoyl-L-asparagine (Example 3, Step A) by process identical to Example 3, Step B to give the title compound in 52% yield; melting point=111°-114° C.; R_{f} (C)=0.44; R_{f} (D)=0.46; NMR (CDCl₃) 1.0-2.2 (m, 19H, decane CH₂ −7,8,9,10, CH-1,6, t-butoxy CH₃); 2.2-3.83 (m, 11H, decane CH₂ −2, 5, butyl CH₂ −1, 4, CH-3); 4.13 (m, 2H, butyl CH-2, OH); 4.95 (m, 1H, asn CH); 5.73, 6.24 (s, s, 2H, NH₂); 6.7-7.33 (m, 6H, aromatic, NH); 7.4-8.42 (m, 6H, aromatic); 9.2 (broad m, 1 H, NH).

### Example 11

### cis-1,6-3-t-Butoxycarbonyl-4-[(2RS,3S)-2-hydroxy-3-[N-(2-pyridyl)-methoxycarbonyl-L-valyl]amino-4-phenylbutyl]-3,4-diaza-bicyclo[4.4.0]decane

Step A: N-(2-Pyridyl)methoxycarbonyl-L-valine: An equimolar mixture of (2-pyridyl)carbinol (3 g) and methyl L-2-isocyanato-3-methylbdtanoate (4.32 g) (Fankhauser P. et al., Helv. Chim. Acta, 1970, 2298-2313) was stirred for 12 hours at 80°-90° C. under nitrogen to give 7.32 g (100%) of N-(2-pyridyl)methoxycarbonyl-L-valine methyl ester as a colorless syrup; NMR (CDCl₃) 0.94 (m, 3H, val CH₃); 2.17 (m, 1H, val CH—); 3.71 (s, 3H, OCH₃); 4.27. (m, 1H, val CH—); 5.18 (s, 2H, CH₂); 5.43 (m, 1H, NH); 6.85-7.82 (m, 3H, aromatic); 8.45 (m, 1 H, aromatic). This was diluted to 25 mL with methanol and 6.04 mL of 5M aqueous potassium hydroxide was added. The resulting mixture was stirred for 1 hour at reflux, then cooled to room temperature and evaporated to dryness in vacuo. The residue was diluted to 25 mL with water and washed with ether. The aqueous phase was cooled in an ice bath and acidified to pH=5 and allowed to stay overnight at 40° C. The resultant precipitate was filtered off; washed with small portions of cold water (3×15 ml) and dried in vacuo over phosphorous pentoxide to give 4.92 g (71% yield) of the title compound melting at 116°-118° C.; NMR (DMSO-d.sub.6) 0.93 (d, 6H, val CH₃); 2.1 (m, 1H, val CH—); 3.4 (broad s, 1H, OH); 3.93 (m, 1H, val CH—); 5.13 (s, 2H, CH₂); 7.17-8.0 (m, 4H, aromatic, NH); 8.5 (m, 1H, aromatic).

Step B: cis-1,6-3-t-Butoxycarbonyl-4-[(2RS,3S)-2-hydroxy-3-[N- (2-pyridyl)methoxycarbonyl-L-valyl]amino-4-phenylbutyl]-3,4-diaza-bicyclo-[4.4.0]decane:

When the product of Step A is substituted for N-quinaldoyl-L-asparagine in Example 10, the identical process afforded the title compound, melting at 82°-87° C., in 38% yield after purification under the conditions given in Example 9; R_{f} (B)=0.08; R_{f} (C)=0.64; R_{f} (D)=0.66,; NMR (CDCl₃) 0.82 (m, 6H, val CH₃); 1.05-2.73 (m, 22H; decane CH₂ −5,7,8,9,10, CH-1,6, t-butoxy CH₃, val CH—); 2.73-4.6 (m, 9H, butyl CH₂ −1,4, CH-2,3, decane CH₂ −2, val CH—); 5.05-5.5 (m, 3H, CH₂, OH); 5.5-6.78 (m, 2H, NH); 7.0-7.9 (m, 8H, aromatic); 8.57 (m, 1H, aromatic).

### Example 12

### cis-1,6-3-t-Butoxycarbonyl-4-[(2RS,3S)-2-hydroxy-3-(N-quinaldoyl-L-glutaminyl)amino-4-phenylbutyl]-3.4-diaza-bicyclo[4.4.0]decane

Step A: N-Quinaldoyl-L-Glutamine: When L-glutamine was substituted for L-valine in Step A of Example 2, the identical process afforded the title compound, melting at 188°-190° C., in 72% yield; NMR (CDCl₃/DMSO-d₆ 1:1) 2.34 (m, 4H, gln CH₂); 4.7 (m, 1H, gln CH—); 6.3, 7.15 (broad ss, 2H, NH₂); 7.4-8.51 (m, 7H, aromatic OH); 8.82(d, 1H, NH).

Step B: cis-1,6-3-t-Butoxycarbonyl-4-[(2RS,3S)-2-hydroxy-3-[N-quinaldoyl-L-glutaminyl]amino-4-phenylbutyl]-3,4-diaza-bicyclo[4.4.o]decane: When the product of Step A is substituted for N-quinaldoyl-L-asparagine in Example 10, the identical process afforded the title compound, melting at 106°-115° C., in 18% yield; R_{f} (C)=0.27; R_{f} (D)=0.30; NMR (CDCl₃) 0.8-2.7 (m, 26H, decane CH₂ −7,8,9,10, CH-1,6, gln CH₂, t-butoxy CH₃, butyl CH-3); 2.7-3.8 (m, 6H, decane CH₂ −2,5, butyl CH₂ −4); 4.36 (m, 1H, butyl CH-2); 4.6 (m, 1H, gln CH); 5.1 (broad s, 1H, OH); 5.4 (m, 1H, NH); 6.07, 6.6 (d,d, 2H, NH₂); 6.8-8.5 (m, 11H, aromatic); 8.8 (m, 1H, gln NH).

### Example 13

### cis-1,6-3-t-Butoxycarbonyl-4-[(2RS,3S)-2-hydroxy-3-(N-quinaldoyl-L-threonyl)-amino-4-phenylbutyl[-3,4-diaza-bicyclo [4.4.0] decane

Step A: N-Quinaldoyl-L-threonine: When L-threonine was substituted for L-valine in Step A of Example 2, the identical process afforded the title compound, melting at 184°-185° C., in 74% yield; NMR (CDCl₃/DMSO-d₆ 1:1) 1.29 (m, 3H, CH₃); 4.5 (m, 1H, thr CH ); 4.68 (dd, 1H, thr CH—); 7.4 -9.27 (m, 9H, aromatic, acid OH, 2-OH, NH).

Step B: cis-1,6-3-t-Butoxycarbonyl-4-[2RS,3S)-2-hydroxy-3-(N-quinaldoyl-L-threonyl)amino-4-phenylbutyl]-3,4-diaza-bicyclo[4.4.0]decane: When the product of Step A is substituted for N-quinaldoyl-L-asparagine in Example 10, the identical process afforded the title compound, melting at 102°-112° C., in 36% yield, R_{f} (C)=0.72; R_{f} (D)=0.61, 0.7; NMR (CDCl₃) 1.0-2.75 (m, 25H, t-butoxy CH₃, decane CH₂ −7,8,9,10, CH-1,6, butyl CH₂ −4, OH); 2.75-4.0 (m, 8H, decane CH₂ −2,5, butyl CH₂ −4, OH); 4.0-4.7 (m, 3H, thr CH—, butyl CH-3); 6.5-7.4 (m, 6H, aromatic, NH); 7.4-8.5 (m, 6H, aromatic); 8.8 (m, 1H, thr NH).

### Example 14

### 2-t-Butoxycarbonyl-3-[(2RS,3S)-2-hydroxy-3-phenylmethoxycarbonyl)amino-4-phenylbutyl]-2,3-diaza-bicyclo[2.2.1 ]hept-5-ene

Step A: 2-t-Butoxycarbonyl-3-phenylmethoxycarbonyl-2,3-diaza-bicyclo-[2.2.1]hept-5-ene: To a stirred mixture of 1 g (4.34 mmol) of 1-benzyloxycarbonyl-2-t-butoxycarbonylhydrazine (Dutta et al., J.C.S. Perkin I, 1975, 1712-1720) in 30 mL of anhydrous methylene chloride 1.55 g (8.7 mmol) of N-bromosuccinimide was added at 0° C. and the stirring was continued for 1 hour with external cooling in an ice bath. The reaction mixture was washed with 10% aqueous sodium thiosulfate solution and saturated aqueous sodium chloride solution, dried over anhydrous magnesium sulfate and evaporated to dryness in vacuo. The residue was redissolved in 15 mL of anhydrous ether, 0.57 g (8.7 mmol) of freshly distilled cyclopentadiene was added and the mixture was allowed to stay for I hour at room temperature. Evaporation to dryness under reduced pressure gave 0.77 g (54% yield) of the title product as a colorless syrup; NMR (CDCl₃) 1.44 (s, 9H, t-butoxy CH₃); 1.7 (m, 2H, CH₂ −7); 5.06 (m, 2H, CH-1,4); 5.15 (s, 2H; methoxy CH₂); 6A (m, 2H, CH-5,6); 7.24 (m, 5H, aromatic).

Step B: 2-t-Butoxycarbonyl-3-[(2RS,3S)-2-hydroxy-3-(phenylmethoxycarbonyl)amino-4-phenylbutyl]-2,3-diaza-bicyclo[2.2.1]-hept-5-ene: A mixture of 0.2 g (0.6 mmol) of the product of Step A and 0.8 mL of 1N aqueous solution of potassium hydroxide in 5 mL of methanol was refluxed under nitrogen for 4 hours. The resulting mixture was partially evaporated, diluted to 10 mL with water and extracted with diethyl ether (3×10 ml). The combined organic phase was washed with saturated aqueous sodium chloride solution, dried over anhydrous magnesium sulfate and evaporated to dryness. The residue was purified by column chromatography (silica gel; hexane/ethyl acetate 3:2) to give 0.05 g (42% yield) of 2-t-butoxycarbonyl-2,3.-diazabicyclo[2.2.1]hept-5-ene. This material (0.049 g, 0.25 mmol) was dissolved in 2 mL of isopropanol containing 0.0744 g (0.25 mmol) of 2(R,S)-3(S)-1,2-epoxy-3-phenylmethoxycarbonylamino-4-phenylbutane (Step A of Example 6) and the resulting mixture was stirred for 15 hours at 80°±50° C. under nitrogen. The mixture was cooled to room temperature, evaporated to dryness in vacuo and purified by column chromatography (silica gel hexane/ethyl acetate 4:1) to give 0.054 g (44% yield) of title product; melting point=111°-113° C.; R_{f} (A)=0.07; R_{f} (B)=0.31; NMR (CDCl₃) 1.43 (s, 9H, t-butoxy CH₃); 1.8 (m, 2H, CH₂ −7); 2.4-3.15 (m, 4H, butyl CH₂ −1,4); 3.2-4.2 (m, 3H, butyl, CH-2,3, OH); 4.5-5.33 (m, 5H, CH-1,4, methoxy CH₂, NH) ; 6.2-6.6 (m, 2H, CH-5,6) ; 7.2 (m, 1OH, aromatic).

### Example 15

### 2-t-Butoxycarbonyl-3-[{2RS,3S)-2-hydroxy-3-(phenylmethoxycarbonyl)amino-4-phenylbutyl]-2,3-diaza-bicyclo[2.2.1 ]heptane

When the product of Step A of Example 14 is substituted for cis-1,6-4-benzyloxy-carbonyl-3-t-butoxycarbonyl-3-4-diaza-bicyclo[4.4.0]decane in Example 8, a similar process afforded the title compound in 31% yield; melting point=119°-126° C.; R_{f} (A)=0.12; R_{f} (B)=0.34, 0.39; NMR (CDCl₃) 1.2-2.1 (m, 15H, t-butoxy CH₃, CH₂ −5,6,7); 2.5-3.2 (m, 4H, butyl CH₂ −1,4); 3.2-4.4 (m, 4H, butyl CH-2,3, CH-1,6); 4.7-5.5 {m, 4H, methoxy CH₂, NH, OH); 7.26 (m, 10H, aromatic).

### Example 16

### 2-t-Butoxycarbonyl-3-[(2RS, 3S)-2-hydroxy-3-[N-(2-pyridyl)-methoxycarbonyl-L-valyl]amino-4-phenylbutyl]-2.3-diaza-bicyclo[2.2.11-heptane

According to Example 2, Step B the product of Example 15 was converted quantitatively to 2-t-butoxycarbonyl-3-[(2RS,3S)-3-amino-2-hydroxy-4-phenylbutyl]-2,3-diaza-bicyclo[2.2.l]heptane. This material was coupled to N-(2-pyridyl)methoxycarbonyl-L-valine (Example 11, Step A) by process identical to Example 3, Step B to give the title compound in 51% yield: melting point=73°-77° C.; R_{f} (C)=0.45; R_{f} (D)=0.49; NMR (CDCl₃) 0.7-1.0 (m, 6H, val CH₃); 1.25-2.15 (m, 16H, t-butoxy CH₃, val CH—, CH₂ −5,6,7); 2.55-3.1 (m, 4H, butyl CH₂ −1,4); 3.3-3.7 (butyl CH-2,3); 3.91 (m, 1H, val CH—); 4.1-4.4 (m, 2H, CH-1,4); 4.9-5.4 [m, 4H, methoxy CH₂ (s, 5.26), OH, NH]; 6.6 (m, 1H, NH); 7.26, 7.7, 8.57 (m, 7H, 1H, 1H, aromatic).

### Example 17

### 2-[N-(1S)(2-methyl-1-methoxycarbonylpropyl)carbamoyl]-3-[(2RS,3S)-2-hydroxy -3-[N-(2-pyridyl)methoxy-L-valyl]amino-4-phenylbutyl]-2,3-diaza-bicyclo[2.2.1]heptane

According to Example 4, Step B, the product of Example 16 was converted quantitatively to the hydrochloride salt of 3-[(2RS, 3S)-2-hydroxy-3-[N-(2-pyridyl)-methoxy-L-valyl]amino-4-phenylbutyl]-2,3-diaza-bicyclo-[2.2.l]heptane. This material (0.06 g; 0.113 mmol) and an equimolar amount of methyl L-2-isocyanato-3-methyl-butanoate were dissolved in 0.4 mL of ethanol free chloroform and to it was added 0.031 mL of diisopropylethylamine. The resulting mixture was allowed to stay for 12 hours at room temperature, under nitrogen, then diluted to 15 mL with ethyl acetate and washed with water and dried over magnesium sulfate.

Evaporation in vacuo and purification by column chromatography (silica gel, ethyl acetate) gave 0.051 g (66%) of the title compound; melting point=79°-84° C., R_{f} (C)=0.2; R_{f} (D)=0.46; NMR(CDCl3); 0.5-1.0 (m, 12H, val CH₃); 1.0-2.5 (m, 1 OH, val CH—, butyl CH₂ −1, CH₂ −5,6,7); 2.5-3.33 (m, 3H, butyl CH₂ −4, CH-3); 3.33-4.05 (m, 6H, val CH—, CH-4, OCH₃); 4.05-5.5 (m, 6H, butyl CH-3, OH, CH-1, NH, methoxy CH₂); 5.82-6.7 (m, 2H, val NH); 6.9-7.9, 8.6 (m, m, 8H, 1H, aromatic).

### Example 18

### 2-t-Butoxycarbonyl-3-[(2RS, 3S)-2-hydroxy-3-(N-quinaldoyl-L-asparaginyl)amino-4-phenylbutyl]-1.2.3.4-tetrahydrophthalazine

Step A: 2-t-Butoxycarbonyl-3-[(2RS,3S)-2-hydroxy-3-(phenylmethoxycarbonyl)amino-4-phenylbutyl]-1,2,3,4-tetrahydrophtalazine: To a mixture of 0.19 g (1.11 mmol) of hydrochloride salt of 1,2,3,4-tetrahydrophthalazine [Groszkowski and Wesolowska, Arch. Pharzm. (Weinheim) 314, 880 (1981)] and 0.23 g (1.05 mmol) of di-tert-butyl dicarbonate in 5 mL of chloroform was added 0.147 mL (1.05 mmol) of triethylamine under nitrogen. After stirring for 5 hours at room temperature the mixture was diluted to 30 mL with ethyl acetate, washed with water and saturated aqueous sodium chloride solution and dried over magnesium sulfate. Evaporation of the solvent in vacuo and purification of the residue by chromatography on silica gel (hexane/ethyl acetate 4:1) gave 0.0921 g (37%) of 2-t-butoxycarbonyl-1,2,3,4-tetrahydrophthalazine; NMR (CDCl₃) 1.5 (s, 9H, t-butoxy CH₃); 4.0 (s, 2H, CH₂ −4); 4.47 (broad s, 1H, NH); 4.64 (s, 2H, CH₂ −1); 6.95 (m, 4H, aromatic). When this material was substituted for 2-t-butoxy-carbonyl-2,3-diazabicyclo[2.2.1]-hept-5-ene in Step B of Example 14 a similar process afforded the title compound in 24% yield after purification on column chromatography (alumina, chloroform/ethyl acetate 95:5); melting point=68°-71° C.; NMR (CDCl₃) 1.5 (s, 9H, t-butoxy CH₃); 2.18-3.15 (m, 4H, butyl CH₂ −1,4); 3.3-5.5 (m, 10H, butyl CH-2,3, CH₂ −1,4, methoxy CH₂, OH, NH); 7.22 (m, 14H, aromatic).

Step B: 2-t-Butoxycarbonyl-3-[(2RS, 3S)-2-hydroxy-3-(N-quinaldoyl-L-asparaginyl)amino-4-phenylbutyl]-1,2,3,4-t etrahydrophthalazine: When the product of Step A is substituted for cis-1,6-3-t-Butoxycarbonyl-4-[(2RS,3S)-2-hydroxy-3-(phenylmethoxycarbonyl)amino-4-phenylbutyl]-3,4-diazabicyclo[4.4.0]decane in Example 10 the identical process afforded the title compound in 70% yield; melting point=108°-112° C.; R_{f} (C)==0.44; R_{f} (D)=0.39; NMR (CDCl₃) 1.47 (m, 9H, t-butyl CH₃); 2.3-3.11 (m, 6H, asn CH₂, butyl CH₂ −1,4); 3.2-5.14 (m, 8H, butyl CH-2,3, asn CH—, CH₂ −1,4, OH); 5.14-6.1 (m, 2H, NH); 6.6-7.4 (m, 10H, aromatic, NH); 7.62, 7.77, 7.87 (3×m, 1H, 1H, 1H, aromatic); 8.1-8.4 (m, 3H, aromatic); 9.11 (m, 1H, asn NH).

### Example 19

### t-Butyl 3p-isopropyl-3-[(2S, 3S)-2-hydroxy-3-(phenylmethoxycarbonyl)-amino-4-phenylbutyl]carbazate

Step A: 2(R)-3(S)-1,2-Epoxy-3-phenylmethoxycarbonylamino-4-phenylbutane: To a stirred solution of 6.02 g (40 mmol) of sodium iodide in 50 mL of anhydrous acetonitrile was added 2.6 mL (22 mmol) of chlorotrimethylsilane under nitrogen. After 10 minutes of stirring, 6 g (20.1 mmol) of the predominantly erythro isomer of 2(R,S)-3(S)-1,2-Epoxy -3-phenylmethoxycarbonylamino-4-phenylbutane (Example 6, Step A) was added and stirring was continued for additional 1 hour. To this mixture was added 4 g (61.2 mmol) of zinc dust followed by 6 mL of acetic acid. The resulting mixture was vigorously stirred for about 5 hours at room temperature and the solid material was removed by filtration. The filtrate was evaporated to dryness in vacuo and the residue was diluted to 75 mL with ether, washed with water and 5N aqueous sodium thiosulfate and dried over anhydrous magnesium sulfate. Evaporation in vacuo and purification by chromatography on silica gel (hexane/ethyl acetate 4:1) gave 5.1 g (90%) of (S)-2-(phenylmethoxycarbonyl)amino-1-phenylbut-3-ene; R_{f} (A)=0.5; melting point=87°-88° C. (hexane); NMR (CDCl₃) 2.87 (d, 2H, butene CH₂ −1); 4.77 (m, 2H, butene CH₂ −4); 5.0 (m, 1H, NCH); 5.06 (s, 2H, methoxy CH₂); 5.18 (broad d, 1H, NH); 5.55-6 (m, 1H, butene CH-3); 7.19, 7.27 (m, s, 5H, SH, aromatic). This material (2.23 g; 7.93 mmol) was dissolved in 25 mL of dry methylene chloride and 4.5 g (22.1 mmol) of 85% 3-chloroperoxybenzoic acid was added at +40° C. The resulting mixture was stirred for two days at the above temperature, then diluted to 50 mL with ether, washed sequentially with 0° C. 10% aqueous sodium sulfite solution, saturated aqueous sodium bicarbonate and saturated aqueous sodium chloride and dried over magnesium sulfate. After evaporation of the solvent the crude product was purified by crystallization from a mixture of hexane/methylene chloride to give 2.1 g (89% yield) of the title epoxide with the predominant threo stereochemistry; melting point=83° -84° C.; NMR (CDCl₃) 2.47 (m, 5H, butane CH₂ −1,4, CH-2); 3.74 (m, 0.15H, NCH); 4.2 (m, 0.85H, NCH); 4.53 (broad d, 1H, NH); 5.03 (m, 2H, methoxy CH₂); 7.3 (m, 10H, aromatic).

Step B: t-Butyl 3-isopropyl-3-[(2S, 3S)-2-hydroxy-3-(phenylmethoxycarbony)-amino-4-phenylbutyl)carbazate: A mixture of 2.03 g (6.83 mmol) of the product of Step A and 1.2 g (7.6 mmol) of t-butyl 3-isopropylcarbazate in 8 mL of isopropanol was stirred for 12 hours at 70°±50° C. under nitrogen. After evaporation of the solvent in vacuo the solid residue was recrystallised from hexane to give 2.6 g (80% yield) of the title compound melting at 114°-115° C.; R_{f} (A)=0.2; R_{f} (B)=0.61; NMR (CDCl₃) 0.95 (m, 6H, isopropyl CH₃); 1.42 (s, 9H, t-butyl CH₃); 2.44 (m, 2H, butyl CH₂ −1); 2.94 (m, 3H, butyl CH₂ −4, CH-3); 3.33-3.93 (m, 2H, isopropyl CH, butyl CH-2); 4.4 (broad m, 1H, OH); 5.05 (s, 2H, methoxy CH₂); 5.33 (broad m, 2H, NH); 7.18, 7.27 (m, s, 5H, 5H, aromatic).

### Example 20

### t-Butyl 3-isopropyl-3-[(2S, 3S)-2-hydroxy-3-(N-quinaldoyl-L-asparaginyl)amino-4-phenylbutyl]carbazate

When the product of Example 19 was substituted for t-butyl 3-isopropyl-[(2R, 3S)-2-hydroxy-3-(phenylmethoxycarbonyl)amino-4-phenylbutyl]carbazate in Example 3, the identical process afforded the title compound in 66% yield; melting point=203°-204° C. (chloroform); R_{f} (C)=0.36; R_{f} (D)=0.37; NMR (5% CD₃OD in CDCl₃); 1.0 (m, 6H, isopropyl CH₃); 1.4 (s, 9H, t-butyl CH₃); 2.53 (d, 2H, butyl CH₂ −1) ; 2.87 (m, 4H, asn CH₂, butyl CH₂ −4); 3.13 (s, 6H, CD₃ OH); 3.42 (m, 2H, isopropyl CH, butyl CH-3); 4.0(m, 1H, butyl CH-2); 4.89 (m, 1H, asn CH—); 7.11 (m, 5H, phenyl); 7.41-8.47 (m, 6H, quinaldoyl).

### Example 21

### cis-1,6-3-t-Butoxycarbonyl-4-[2S, 3S)-2-hydroxy-3-(phenylmethoxycarbonyl)amino-4-phenylbutyl]-3,4-diaza-bicyclo[4.4.0]decane

When the product of Step A, Example 8, is substituted for t-butyl 3-isopropyl-carbazate in Example 19, Step B, the identical process afforded the titled compound in 78%; melting point=110°-111° C. (hexane); R_{f} (A)=0.28; R_{f} (B)=0.63; NMR (CDCl₃) 1.0-2.18 (m, 19H, decane CH₂ −7,8,9,10, CH-1,6, t-butoxy CH₃); 2.4 (m, 2H, decane CH₂ −5); 2.75-4.1 (m, 8H, decane CH₂ −2, butyl CH2-1,4, CH-2,3); 4.93 (broad s, 1H, OH); 5.07 (s, 2H, methoxy CH₂); 5.31 (broad m, 1 H, NH); 7.22, 7.32 (m, s, 5H, 5H, aromatic).

### Example 22

### cis-1,6-3-t-Butoxycarbonyl-4-[(2S, 3S)-2-hydroxy-3-amino-4-phenylbutyl]-3,4-diaza-bicyclo[4.4.0]decane

According to the method of Example 2, step B, the product of Example 21 (2 g; 0.037 moll was converted quantitatively to the title compound (1.5 g of a heavy syrup); NMR (CDCl₃): 1.0-2.32 (m, 19H, decane CH₂ −7,8,9,10, CH-1,6, t-butoxy CH₃); 2.32-4.54 (m, 13H, butyl CH₂ −1,4, CH-2,3, decane CH₂ −2,5, NH₂, OH); 7.28 (m, 5H, aromatic)

A fractional crystallisation of the above product from hexane gave 0.74 g of isomer A as a colorless solid melting at 123°-124° C.; NMR (CDCl₃) 1.0-2.25 (m, 21H, decane CH₂ −7,8,9,10, CH-1,6, t-butoxy CH₃, NH₂); 2.35-3.0 (m, 5H, butyl CH₂ −1,4, CH-3); 3.05-3.4 (m, 3H, butyl CH-2, decane CH₂ −5); 3.5 (m, 2H, decane CH₂ −2); 3.82 (d, 1H, OH); 7.27 (m, 5H, aromatic).

The hexane fraction gave 0.76 g of isomer B, after evaporation of the solvent. This was purified by column chromatography (silica gel, 8% methanol in methylene chloride; R_{f}=0.16) to give 0.72 g of pure isomer B as a colorless syrup: NMR (CDCl₃) 1.0-2.4 (m, 21H, decane CH₂ −7,8,9,10, CH-1,6, t-butoxy CH₃, NH₂); 2.4-3.1 (m, 6H, butyl CH₂ −1,4, CH-2,3); 3.22-3.4 (m, 2H, decane CH₂ −5); 3.52 (m, 2H, decane CH₂ −2); 3.76 (d, 1 H, OH); 7.27 (m, 5H, aromatic).

### Example 23

### cis-1,6-3-t-Butoxycarbonyl-4-[(2S, 3S)-2-hydroxy-3-(N-quinaldoyl-L-asparaginyl)amino-4-phenylbutyl]-3,4-diaza-bicyclo[4.4.0]decane

When the product of Example 22 (mixture of isomers A and B) was substituted for cis-1,6-3-t-butoxycarbonyl-4-[(2RS,3S)-2-hydroxy-3-amino-4-phenylbutyl]-3,4-diaza-bicyclo[4.4.0]decane in Example 10, the identical process afforded the title compound in 72% yield; melting point=108°-110° C., R_{f} (C)=0.44; R_{f} (D)=0.46; NMR (CDCl₃) 0.71-2.18 (m, 19H, decane CH₂ −7,8,9,10, CH-1,6, t-butoxy CH₃); 2.18-4.48 (m, 12H, asn CH₂, decane CH₂ −2,5, butyl CH₂ −1,4, CH-2,3); 4.95 (m, 2H, ash CH, OH); 5.55, 6.13 (broad m,m, 2H, NH); 6.84-7.4 (m, 6H, aromatic, NH); 7.4-8.39 (m, 6H, aromatic); 9.22 (m, 1 H, NH).

A sample of this product was separated to two isomers by reverse phase (Whatman C₈ semipreparative column) high pressure liquid chromatography, using 37% of 0.1% aqueous solution of trifluoroacetic acid in acetonitrile containing 0.07% of trifluoroacetic acid and 10% of water, for the elution: Isomer A, R_{f}=16.8 min.; Isomer B, R_{f}=18.3 min.

When the isomers A and B of the product of Example 22 were used instead of mixture, the respective isomers of the title compound were obtained.

Isomer A: 69% yield; melting point=110°-116° C.; NMR (CDCl₃): 1.0-1.8 (m, 19H, t-butyl CH₃, decane CH₂ −7,8,9,10, CH-1,6); 2.2-2.6 (m, 2H, butyl CH₂ −1); 2.7-3.3 (m, 7H, asn CH₂, butyl CH₂ −4, CH-3, decane CH₂ −5); 3.56 (m, 2H, decane CH₂ −2); 4.07 (m, 1H, butyl CH-2); 5.0 (m, 1H, asn CH); 5.4-5.75 (m, 2H, NH, OH); 6.1 (m, 1H, NH); 7.14 (m, 6H, aromatic, NH); 7.63, 7.8, 8.22 (m, m, m, 1H, 2H, 3H, aromatic); 9.21 (m, 1H, asn NH).

Isomer B: 78% yield; melting pont=122°-126° C.; NMR (CDCl₃): 1.1-1.71 (m, 19H, t-butyl CH₃, decane CH₂ −7,8,9,10, CH-1,6); 2.2-2.6 (m, 2H, butyl CH₂ −1); 2.7-3.15 (m, 6H, asn CH₂, butyl CH₂ −4 decane CH₂ −5); 3.43 (m, 3H, butyl CH-3, decane CH₂ −2); 4.1 (m, 1H, butyl CH-2); 4.94 (m, 1H, OH); 5.0 (m, 1H, asn CH); 5.55, 6.2 (m, m, 1H, 1H, NH₂) ; 7.14 (m, 6H, aromatic, NH) ; 7.63, 7.8, 8.22 (m, m, m, 1H, 2H, 3H, aromatic); 9.27 (m, 1 H, asn NH).

### Example 24

### 1-Trimethylacetyl-2-[(2S,3S)-2-hydroxy-3-1phenylmethoxycarbonyl)amino-4-phenylbutyl]-2-isopropylhydrazine

Step A: 1-trimethylacetyl-2-isopropylhydrazine: A mixture of 10 g (0.086 mol) of methyl trimethylacetate and 3.2 g (0.1 mol) of anhydrous hydrazine was refluxed for 12 hr. then evaporated to dryness under reduced pressure. The residue was purified by crystallization from an ether/hexane mixture to give 9 g (90% yield) of trimethylacetylhydrazide, melting at 190°-191° C. When this product is substituted for t-butyl carbazate in Step A of Example 1 the identical process afforded the title compound in 67% yield, as colorless crystals; NMR (CDCl₃) 1.03 (d, 6H, isopropyl CH₃) 1.18 (s, 9H, trimethyl CH₃); 3.07 (m, 1 H, isopropyl CH); 4,62 (broad s, 1 H, NH); 7.4 (broad s, 1 H, NH amide).

Step B: 1-trimethylacetyl-2-[2S,3S)-2-hydroxy-3-(phenylmethoxycarbonyl)amino-4-phenylbutyl]-2-isopropyl-hydrazine: When the product of Step A was substituted for t-butyl 3-isopropylcarbazate in Step B of Example 19, the identical process afforded the title compound in 69% yield; melting point=132°-134° C.: R_{f}(A)=0.07; R_{f}(B)=0.33; NMR (CDCl₃) 0.72-1.3 (m, 15H, isopropyl CH₃, t-butyl CH₃); 2.1-3.16 (m, 5H, butyl CH₂ −1,4, CH-3); 3.16-4.0 (m, 2H, butyl CH-2, isopropyl CH); 4.86 (s, 1H, OH); 5.08 (s, 2H, methoxy CH₂); 5.4 (d, 1H, NH); 6.1 (s, 1H, NH); 7.2, 7.31 (m, s, 5H, 5H aromatic).

### Example 25

### 1-Trimethylacetyl-2-[(2S,3S)-2-hydroxy-3-(N-quinaldoyl-L-asparaginyl(amino-4-phenylbutyl]-2-isopropylhydrazine

When the product of Example 24 was substituted for t-butyl-3-isopropyl-[(2R,3S)-2-hydroxy-3-(phenylmethoxycarbonyl)amino-4-phenylbutyl]carbazate in Example 3, the identical process afforded the title compound in 65% yield; melting point=222°-223.5° C.; R_{f} (C)=0.1; R_{f} (D)=0.49; NMR (10% CD₃OD in CDCl₃): 0.7-1.31 (m, 15H, trimethyl CH₃, isopropyl CH₃); 2.0-3.6 (m, 9H, asn CH₂, butyl CH₂ −1,4, CH-2,3, isopropyl CH); 4.05 (s, CD₃ OH), 5.0 (m, H, asn CH); 6.64-8.5 (m, 11 H, aromatic).

### Example 26

### 1-(t-Butylamino)carbonyl-2-[(2S,3S)-2-hydroxy-3-(N-quinaldoyl-L-asparaginyl)amino-4-phenylbutyl]-2-isopropylhydrazine

To a vigorously stirred mixture of 0.33 g (0.0103 mol) of anhydrous hydrazine in 50 mL of dry ether was added 1 g (0.01 mol) of t-butyl isocyanate. The resulting mixture was stirred for 2 hr. at room temperature then was kept overnight at 4° C. The crystals formed were filtered off, washed with a small portion of ether and dried to give 0.94 g (72% yield) of (t-butylamino)carbonylhydrazine melting at 192°-193° C. When this was substituted for t-butyl carbazate in Step A of Example 1, the identical process afforded 1-(t-butylamino)carbonyl-2-isopropylhydrazine in 58% yield as a white solid; NMR (CDCl₃): 1.03 (d, 6H, isopropyl CH₃); 1.33 (s, 9H, t-butyl CH₃); 3.9 (broad s, 1 H, NH); 6.02 (broad s, 2H, NH amide). When this was substituted for t-butyl 3-isopropylcarbazate in step B of Example 19 the identical process afforded 1-(t-butylamino)carbonyl-2-[(2S,3S)-2-hydroxy-3-(phenylmethoxycarbonyl)amino-4-phenylbutyl]-2-isopropylhydrazine in 68% yield, as a white solid; NMR (CDCl₃) : 1.0 (m, 6H, isopropyl CH₃); 1.3 (s, 9H, t-butyl CH₃); 2.33-4.22 (m, 8H, butyl CH₂ −1,4, CH-2,3, OH, isopropyl CH); 5.05 (s, 2H, methoxy CH₂); 5.3 (m, 2H, NH); 5.91 (m 1H, NH); 7.2, 7.35 (m, s, 5H, 5H, aromatic). When this was substituted for t-butyl 3-isopropyl-[(2R, 3S)-2-hydroxy-3-(phenylmethoxycarbonyl)amino-4-phenylbutyl]carbazate in Example 3, the identical process afforded the title compound in 67% yield; melting point=119°-125° C.; R_{f} (C)=0.06; R_{f}=0.43; NMR(CDCl₃): 1.0 (m, 6H, isopropyl CH₃); 1.32 (s, 9H, t-butyl CH₃); 2.24-3.38 (m, 7H, butyl CH₂ −1,4, CH-3, asn CH₂); 3.38-4.63 (m, 3H, butyl CH-2, OH, isopropyl CH); 5.09 (m, 1 H, asn CH); 5.63-8.4 (m, 16H, aromatic, NH); 9.0 (d, 1H, asn NH).

### Example 27

### t-Butyl 3-isopropyl-3-[(2S, 3S)-2-hydroxy-3-(N-picolinoyl-L-asparaginyl)amino-4-phenylbutyl]carbazate

STEP A: N-picolinoyl-L-asparagine: When picolinic acid was substituted for quinaldic acid in Step A of Example 3, the identical process afforded the title compound melting at 171°-172° C., in 68% yield, NMR(DMSO-d₆) 2.75 (m, 2H, asn CH₂); 4.8 (m, 1H, asn CH) ; 6.7-8.8 (m, 6H, aromatic, NH₂) ; 9.0 (d, 1H, NH) 12.7 (broad s, 1 H, OH).

STEP B: t-Butyl 3-isopropyl-3-[2S,3S)-2-hydroxy-3-(N-picolinolyl-L-asparaginyl)amino-4-phenylbutyl]carbazate; When the product of Step A was substituted for N-quinaldoyl-L-aspargine in Example 20, the identical process afforded the title compound in 58% yield; melting point=101°-108° C.; R_{f}(C)=0.16; R_{f} (D)=0.48; NMR (CDCl₃): 1.0 (m, 6H, isopropyl CH₃); 1.4 (s, 9H, t-butyl CH₃); 2.15-3.23(m,7H, butyl CH₂ −1,4, CH-3, asn CH₂; 3.23-4.53 (m, 3H, butyl CH-2, isopropyl CH, OH); 4.94 (m, 1H, asn CH); 5.1-6.41 (m, 3H, NH); 6.7-8.7 (m, 10H, aromatic, NH); 9.05 (m, 1H, asn NH).

### Example 28

### t-Butyl 3-isopropyl-3-[(2S,3S)-2-hydroxy-3-(N-(2-pyridyl)methoxycarbonylanthranilo yl) amino-4-phenylbutyl]carbazate

When the product of Step A of Example 4 was substituted for N-quinaldoyl-L-asparagine in Example 20, the identical process afforded the title compound in 61% yield; melting point=155-157° C.; R_{f} (C)=0.79; R_{f} (D)=0.78; NMR (CDCl₃): 1.0 (m, 6H, isopropyl CH₃); 1.42 (s, 9H, t-butyl CH₃); 2.33-3.22 (m, 5H, butyl CH₂ −1,4 CH-2); 3.62 (m, 1H, butyl CH-3); 4.25 (m, 1H, isopropyl CH); 4.67 (broad s, 1H, OH); 5.3 (s, 2H, methoxy CH₂); 6.52-8.44 (m, 15H, aromatic, NH); 8.55 (m, 1H, NH).

### Example 29

t-Butyl 3-benzyl-3-t(2S,3S)-2-hydroxy-3-(phenylmethoxycarbonyl)amino-4-phenylbutyl]carbazate

STEP A: t-Butyl 3-benzylcarbazate: When benzaldehyde was substituted for acetone in Step A of Example 1, the identical process afforded the title compound in 69% yield as a heavy colorless syrup; NMR (CDCl₃): 1.44 (s, 9H, t-butyl CH₃); 3.63 (broad s, 1H, NH); 4.0 (s, 2H, CH₂); 6.08 (s, 1H, NH); 7.3 (s, 5H, aromatic).

Step B: t-Butyl 3-benzyl-3-[(2S,3S)-2-hydroxy-3-(phenylmethoxycarbonyl)amino-4-phenylbutyl]carbazate: When the product of Step A was substituted for t-butyl 3-isopropyl carbazate in Step B of Example 19, the identical process afforded the title compound in 72% yield; melting point=142°-143° C.; R_{f} (A)=0.16; R_{f} (B)=0.59; NMR (CDCl₃) 1.31 (s, 9H, t-butyl CH₃); 2.12-3.12 (m, 5H, butyl CH₂ −1,4, CH-3); 3.35-4.11 (m, 3H, benzyl CH₂, butyl CH-2); 4.41 (broad s, 1H, OH); 5.05 (s, 2H, methoxy CH₂); 5.2 (m, 2H, NH); 7.22 (m, 15H, aromatic).

### Example 30

### t-Butyl 3-benzyl-3-E(2S,3S)-2-hydroxy-3-(N-quinaldoyl-L-asparaginyl)amino-4-phenylbutyl]carbazate

When the product of Example 29 was substituted for t-butyl 3-ispropyl-[(2S, 3S)-2-hydroxy-3-(phenylmethoxycarbonyl)amino-4-phenylbutyl]carbazate in Example 20, the identical process afforded the title compound in 71% yield; melting point=150°-153° C.; R_{f} (C)=0.38; R_{f} (D)=0.53; NMR (CDCl₃): 1.3 (s, 9H, t-butyl CH₃); 2.13-3.2 (m, 7H, butyl CH₂ −1,4, CH-3, asn CH₂); 3.2-4.73 (m, 4H, benzyl CH₂, butyl CH-2, OH); 5.0 (m, 1H, asn CH); 5.14-6.7 (m, 4H, NH); 6.7-8.35 (m, 16H aromatic); 9.25 (broad m, 1 H, asn NH).

### Example 31

### t-Butyl 3-cyclohexyl-3-[(2S, 3S)-2-hydroxy-3-(phenylmethoxycarbonyl)amino-4-phenylbutyl]carbazate

Step A: t-Butyl 3-cyclohexylcarbazate: When cyclohexanone was substituted for acetone in Step 1 of Example 1, the identical process aforded the title compound in 59% yield as a colorless solid; NMR (CDCl₃): 0.75-2.2 (m, 19H, t-butyl CH₃, cyclohexyl CH₂); 2.75 (m, 1H, cyclohexyl CH); 3.75 (broad s, 1H, NH); 6.27 (broad s, 1H, NH).

Step B: t-Butyl 3-cyclohexyl-3-[(2S,3S)-2-hydroxy-3-(phenylmethoxycarbonyl)amino-4-phenylbutyl]carbazate: When the product of Step A was subsituted for t-butyl 3-isopropyl carbazate in Step B of Example 18, the identical process afforded the title compound in 76% yield; melting point=142°-143° C.; R_{f} (A)=0.28; R_{f} (B)=0.7; NMR (CDCl₃): 0.73-2.0 (m, 19H, t-butyl CH₃, cyclohexyl CH₂); 2.53 (m, 3H, butyl CH₂ −1, CH-3); 3.0 (d, 2H, butyl CH₂ −4); 3.35-4.0 (m, 2H, butyl CH-2, cyclohexyl CH); 4.49 (broad s, 1H, OH); 5.13 (s, 2H, methoxy CH₂); 5.35 (m, 2H, NH); 7.3, 7.4. (m, s, 5H, 5H, aromatic)

### Example 32

### t-Butyl 3-cyclohexyl-3-[(2S.3S)-2-hydroxy-3-(N-quinaldoyl-L-asparaginyl)amino-4-phenylbutyl]carbazate

When the product of Example 31 was substituted for t-butyl 3-isopropyl-3-[(2S, 3S)-2-hydroxy-3-(phenylmethoxycarbonyl)amino-4-phenylbutyl]-carbazate in Example 20, the identical process afforded the title compound in 75% yield: melting point=140°-144° C.; R_{f} (C) 0.42; R_{f} (D)=0.56; NMR (CDCl₃): 0.7-2.17 (m, 19H, t-butyl CH₃, cyclohexyl CH₂); 2.17-3.29 (m, 7H, butyl CH₂ −1,4, CH₃ asn CH₂); 3.3-4.87 (m, 3H, butyl CH-2, cyclohexyl CH, OH): 4.95 (m, 1H, asn CH); 5.14-6.4 (m, 3H, NH); 6.62-8.3 (m, 12H, aromatic, NH); 9.15(d, 1H, asn NH).

### Example 33

### t-Butyl 3-isopropyl-3-[(2S,3S)-2-hydroxy-3-(N-(1-carbamoylmethyl)acryloyl)-amino-4-phenylbutyl]carbazate

STEP A: (1-Carbamoylmethyl)acrylic acid: To a mixture of 3 g (0.027 mol) of itaconic anhydride in 30 mL of tetrahydrofuran, 3 mL of 28% ammonium hydroxide was added. After 1 hr. the reaction mixture was evaporated to dryness under reduced pressure. The residue was dissolved in 15 mL of water, then acidified to pH 2 with concentrated hydrochloric acid and allowed to stay overnight at 40° C. The precipitate formed was filtered off, washed with a small portion of cold water and dried to give 1.4 g (40% yield) of the title compound melting at 153°-154° C.; NMR (DMSO-d₆): 3.11 (s, 2H, CH₂); 5.67,6.13 (s, s, 1H, 1H, CH); 6.7, 7.9 (broad s, s 1H, 1H, NH); 12.15 (broad s, 1 H, OH).

STEP B: t-Butyl 3-isopropyl-3-[(2S,3S)-2-hydroxy-3-(N-( 1-carbamoylmethyl)acryloyl)amino-4-phenylbutyl]carbazate: When the product of Step A was substituted for N-quinaldoyl-L-asparagine in Example 20, the identical process afforded the title compound in 61% yield; melting point=118°-122° C.; R_{f} (C)=0.27; R_{f} (D)=0.49; NMR (CDCl₃): 1.0 (m, 6H, isopropyl CH₃); 1.4 (s, 9H, t-butyl CH₃); 2.49 (m, 2H, butyl CH₂ −1); 3.0 (m, 3H, butyl CH₂ −4, CH-3); 3.2 (s, 2H, methyl CH₂); 3.6 (m, 1H, isopropyl CH); 4.07 (m, 1H, butyl CH-2); 4.6 (broad s, 1H, OH); 5.2-5.8 (m, 4H, acryl CH, NH); 6.4-7.0 (m, 2H, NH₂); 7.2 (m, 5H, aromatic):

### Example 34

### t-Butyl 3-isopropyl-3-[(2S,3S)-2-hydroxy-3-(N-2-(RS)-3-tert-butylthio-2-carbamoylmethylpropionyl)amino-4-phenylbutyl]carbazate

To a mixture of 0.057 g (0.127 mmol) of the product of Example 33 and 0.0172 mL (0.152 mmol) of tert-butyl mercaptan in 0.5 mL of anhydrous methanol, 1 drop of a freshly prepared 20% solution of sodium methoxide in methanol was added. After stirring for 12 hr. at room temperature the mixture was evaporated to dryness, then diluted to 10 mL with ether and washed with water and saturated sodium chloride solution. After drying over anhydrous magnesium sulfate, the ether was evaporated under reduced pressure. The residue was purified by column chromatography (silica gel; ethyl acetate), to give 0.032 g (47% yield) of the title compound; melting point=116°-120° C.; R_{f} (C)=0.42; R_{f} (D)=0.56; NMR (CDCl₃); 0.6-1.63 (m, 24H) t-butyl CH₃, isopropyl CH₃); 2.0-4.47 (m, 13H, butyl CH₂ −1,4, CH-2,3, isopropyl CH, methyl CH₂, propionyl CH₂, CH, OH); 4.82-6.78 (m, 4H, NH₂, NH); 7.11 (m, 5H, aromatic)

### Example 35

### t-Butyl 3-isopropyl-3-[(2S, 3S)-2-hydroxy-3-(N-benzoyl-L-asparaginyl)amino-4-phenylbutyl]carbazate

Step A: N-Benzoyl-L-asparagine: To a vigorously stirred solution of 2 g (0.013 mol) of L-asparagine monohydrate and 2.02 g (0.014 mol) of potassium carbonate in 15 mL of water, 1.51 mL (0.013 mol) of benzoyl chloride was added dropwise, over a period of 15 min., at room temperature. The stirring was continued for 2 hour, then the mixture was extracted with 10 mL of ether and the aqueous phase was acidified to pH 2 with concentrated hydrochloric acid. The white precipitate was filtered off, washed with water and purified by crystallization from isopropyl alcohol to give 2.1 g (68% yield) of the title compound at 190°-192° C.; NMR (DMSO-d₆): 2.62 (m, 2H, CH₂); 3.32 (broad s, 1H, OH); 4.72 (m, 1H, CH); 6.64-8.0(m. 7H, aromatic, NH₂); 8.6 (d, 1H, NH).

Step B: t-Butyl 3-isopropyl-3-[(2S,3S)-2-hydroxy-3-(N-benzoyl-L-asparaginyl)-amino-4-phenylbutyl]carbazate: When the product of Step A was substituted for N-quinaldoyl-L-asparagine in Example 20, the identical process afforded the title compound in 65% yield; melting point=182°-185° C.; R_{f} 0.22; R_{f} (D)=0.51; NMR (CDCl₃/DMSO-d₆, 1:1): 0.92 (m, 6H, isopropyl CH₃); 1.38 (s, 9H, t-butyl CH₃); 2.19-3.11 (m, 7H, butyl CH₂ −1, 4, CH-3, asn CH₂); 3.11-4.57 (m, 3H, isopropyl CH, butyl CH-2, OH); 4.83 (m, 1H, asn CH); 6.5-8.17 (m, 14H, aromatic NH); 8.56 (m, 1H, asn NH).

### Example 36

### 1-t-Butyloxycarbonyl-2-[(2S,3S)-2-hydroxy-3-(phenylmethoxycarbonyl)amino-4-phenylbutyl]hexahydropyridazine

Step A: 1-t-butyloxycarbonylhexahydropyridazine: When 1,4-dibromobutane was substituted for cis-1,2-cyclohexanedimethyliodide in Step A of Example 8, the identical process afforded 1-t-butoxycarbonyl-2-phenylmethoxycarbonylhexahydropyridazine in 65% yield; melting point=71°-72° C.; NMR (CDCl₃)1.15-1.9 (m, 13H, t-butyl CH₃; CH₂ −4,5); 3.0, 4.15 (broad m, m, 2H, 2H, CH₂ −3,6); 5.2 (m, 2H, methoxy CH₂); 7.35 (s, 5H, aromatic). This was converted to the title compound in 93% yield by hydrogenolysis, performed as described in Example 2. The product was isolated as a colorless syrup.

Step B: 1-t-butyloxycarbonyl-2-[(2S,3S)-2-hydroxy-3-(phenylmethoxycarbonyl)amino-4-phenylbutyl)hexahydropyridazine: When the product of Step A was substituted for t-butyl 3-isopropylcarbazate in Step B of Example 19 the identical process afforded the title compound in 71% yield, as a heavy colorless syrup; NMR (CDCl₃) 1.0-1.87 (m, 13H, t-butyl CH₃, pyridazine CH₂ −4,5); 2.0: 4.0 (m, 11 H, butyl CH₂ −1,4, CH-2,3, pyridazine CH₂ −3,6, OH); 5.05 (s, 2H, methoxy CH₂); 5.47(d, 1H, NH); 7.19 (m, 10H, aromatic).

### Example 37

### 1-t-Butyloxycarbonyl-2- [(2S,3S)-2-hydroxy-3-(N-quinaldoyl-L-asparaginyl)-amino-4-phenylbutyl]hexahydropyridazine

When the product of Example 36 was substituted for t-butyl 3-isopropyl-[(2S,3S)-2-hydroxy-3-(phenylmethoxycarbonyl)amino-4-phenylbutyl]carbazate in Example 20, the identical process afforded the title compound in 65% yield; melting point=104°-110° C.; R_{f} (C)=0.3; R_{f} (D)=0.62; NMR (CDCl₃) 1.0-2.04 (m, 13H, t-butyl CH₃, pyridazine CH₂ −4,5); 2.15-4.31 (m, 13H, butyl CH₂ −1,4, CH-2,3, asn CH₂, pyridazine CH₂ −3,6, OH); 4.95 (m, 1H, asn CH); 5.14-6.6 (m, 3H, NH); 6.8-8.4 (m, 11H, aromatic); 9.21 (d, 1 H, asn NH).

### Example 38

### cis-1,6-3-t-Butoxycarbonyl-4-[(2S,3S)-2-hydroxy-3-(N-quinaldoyl-3-cyano-L-alanyl)amino-4-phenylbutyl]-3,4-diaza-bicyclo[4.4.0]decane

Step A: N-Quinaldoyl-3-cyano-L-alanine: To a mixture of 0.198 g (0.69 mmol) of N-quinaldoyl-L-asparagine and 0.24 mL (1.38 mmol) of N,N-diisopropylethylamine in 1 mL of chloroform was added 0.146 g (0.71 mmol) of dicyclohexylcarbodiimide. The reaction mixture was stirred for 24 hr. at room temperature, then partitioned between 10 ml of 5% sodium bicarbonate and 10 mL of ether. The aqueous phase was acidified to pH2 and the acid was taken up by extraction with chloroform (3×10 mL). The organic phase was dried over anhydrous magnesium sulfate, filtered and evaporated to give 0.101 g of crude product. This was recrystallized from a small portion of methylene chloride to give 0.06 g of the title compound melting at 144°-146° C.; NMR (5% DMSO-d₆ in CDCl₃): 3.22 (d, 2H, ala CH₂); 4.95 (m, 1H, ala CH); 7.2-8.57 (m, 7H, aromatic, OH); 9.19(d, 1H, NH).

Step B: cis-1,6-3-t-Butoxycarbonyl-4-[(2S,3S)-2-hydroxy-3-(N-quinaldoyl-3-cyano-L-alanyl)amino-4-phenylbutyl]-3,4-diaza-biyclo[4.4.0]decane: When the product of Step A was substituted for N-quinaldoyl-L-asparagine in Example 22 (isomer A) the identical process afforded the title compound with 67% yield, melting at 106°-112° C.; R_{f} (C)=0.87; R_{f} (D)=0.89; NMR (CDCl₃) 0.7-2.84 (m, 24H, t-butyl CH₃, decane CH₂ −7,8,9,10, CH-1,6, butyl CH₂ −1, CH-3, cyanoalanyl CH₂); 2.85-4.65 (m, 8H, butyl CH₂ −4, CH-2, decyl CH₂ −2,5. OH); 4.7-5.6 (broad m, 2H, cyanoalanyl CH, NH); 6.9-8.5 (m, 11H, aromatic); 8.9 (broad m, 1H, NH).

While the foregoing specification teaches the principles of the present invention, with examples provided for the purpose of illustration, it will be understood that the practice of the invention emcompasses all of the usual variations, adaptations, or modifications, as come within the scope of the following claims and its equivalents.

1. A method of treating or preventing Alzheimer's disease in a subject in need of such treatment comprising administering a therapeutically effective amount of a compound of Formula (I) or a pharmaceutically acceptable salt thereof: 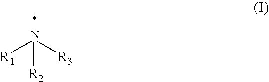
wherein R₁ is selected from R,
wherein R is selected from the group consisting of hydrogen, —R′H, —R′C(O)OR″, —R′C(O)NH₂, —R′C(O)NHR″, —R′C(O)NR″R′″, —R′NHC(O)R″, —R′NR′″C(O)R″, and —R′C(O)R″,
where R″ and R′″ are independently selected from (C₁-C₁₈)alkyl; (C₃-C₁₈) cycloalkyl; (C₃-C₁₈)cycloalkyl(C₁-C₁₈)alkyl; (C₆-C₂₄)aryl; (C₇-C₂₅)aralkyl; (C₂-C₁₈)alkenyl; (C₈-C₂₆)aralkenyl; (C₂-C₁₈) alkynyl; (C₈-C₂₆)aralkynyl; or heterocyclic; all optionally substituted,
where R′ is a divalent radical derived from (C₁-C₁₈)alkyl; (C₃-C₁₈)cycloalkyl; (C₃-C₁₈)cycloalkyl(C₁-C₁₈)alkyl; (C₆-C₂₄)aryl; (C₇-C₂₅)aralkyl; (C₂-C₁₈)alkenyl; (C₈-C₂₆)aralkenyl; (C₂-C₁₈) alkynyl; (C₈-C₂₆)aralkynyl; or heterocyclic; all optionally substituted;
and the moeity 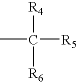
where R₄, R₅ and R₆ are independently a group R as defined above; or R₄ has the meaning of R as defined above and R₅ and R₆ taken together are ═O, ═S, ═NH or ═NR;
R₂ is 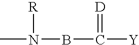
where R is as previously defined;
D is O or S;
Y is selected from hydrogen, —R or —OR, and an amino acid, aza-amino acid or peptide residue in which any functional group present is optionally protected; and
B is optionally absent or is (C₁-C₆)alkylidene, wherein any one or more —CH₂— groups may be replaced by —NR—, —NH—, —O— or —S—, provided that the compound of Formula (I) does not contain a chain of three or more atoms which are not carbon, and wherein any H atom may be substituted by a group R as previously defined;
N*, N, R₁ and R can be optionally taken together to form a cyclic diazaalkane of the formula: 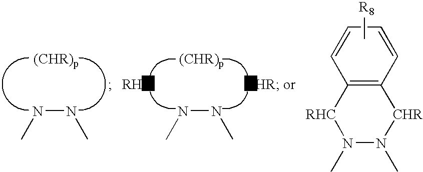
where p is 1 to 3,
each R is independently as defined above, and
R₈ is selected from R, —NH₂, —NHR, —NR₂, —COOH, —COOL, —CHO, —C(O)R, —CN, halo, —CF₃, —OL, —SR, —S(O)R, —S(O)₂R, —CONH₂, —CONHR, —CONR₂, —NHOH, —NHOL, —NO₂, ═O, ═S or —NHNH₂,
wherein each R is independently as defined above, and wherein L is independently R or a hydroxyl protecting group;
or
R₂, N* and R₄ together form a saturated or unsaturated cyclic, bicyclic or fused ring system which may be additionally substituted by —C(O)Y, where Y is as previously defined;
R₃ is X—W-A′-Q-A-, wherein: A′ and A independently are absent or (C₁-C₈)alkylidene, which may be substituted with one or more substituents R as previously defined;
Q is 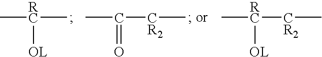
where L and each R, independently of the others, are as previously defined, and optionally Q and A together, or Q and A′ together, or A′, Q and A together form part of a saturated or unsaturated cyclic, bicyclic or fused ring system;
W is absent, or is selected from N(R), O or S,
wherein R is as previously defined; and
X is selected from hydrogen, X₁, where X₁ is Ra— or RbC(O)— or RbS(O)_{z}—,
where z is 1 or 2,
where Ra and Rb are independently (C₁-C₁₈)alkyl; (C₃-C₁₈)cycloalkyl; (C₃-C₁₈)cycloalkyl(C₁-C₁₈)alkyl; heterocyclic; (C₁-C₁₈)alkylheterocyclic; heterocyclic(C₆-C₂₄)aryloxy; (C₁-C₁₈)alkoxy; (C₁-C₁₈)alkoxy(C₁-C₁₈)alkyl; (C₁-C₁₂)alkyl; (C₆-C₂₄)aryloxy(C₁-C₁₈)alkyl; (C₆-C₂₄)aryloxy(C₁-C₁₈) alkoxy; (C₆-C₂₄)aryl; (C₆-C₂₄)aryl (C₁-C₁₈)alkyl; (C₆-C₂₄)aryl(C₁-C₁₈)alkylheterocyclic; (C₁-C₁₂) alkylheterocyclic; heterocyclicoxy(C₁-C₁₈)alkyl; (C₁-C₁₈)alkylamino; di(C₁-C₁₈)alkylamino; (C₆-C₂₄)arylamino; di(C₆-C₂₄) arylamino; (C₇-C₂₅)aralkylamino; or di(C₇-C₂₅)aralkylamino; any of which may be optionally substituted with one or more groups selected from —F, —Cl, —Br, —I, —CF₃, —OH, —OR_{IV}, —NH₂, —NHR_{IV}, —NR_{IV}R_{V}, —CN, —NO₂, —SH, —SR_{IV}, —SOR_{IV}, —SO₂R_{IV}, ═O, ═S, ═NOH, ═NOR_{IV}, —NHOH, —NHOR_{IV}, —CHO, where R_{IV} and R_{V} are independently (C₁-C₁₈)alkyl; (C₃-C₁₈)cycloalkyl; (C₃-C₁₈)-cycloalkyl(C₁-C₁₈)alkyl; (C₆-C₂₄)-aryl; (C₇-C₂₅)aralkyl; (C₂-C₁₈)alkenyl; (C₈-C₂₆)aralkenyl; (C₂-C₁₈)alkynyl; (C₈-C₂₆)-aralkynyl; or heterocyclic; and Re,
where Re is a group of the formula: 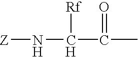
where Z has the meaning of Ra or Rb or is an acylated amino acid, azaamino acid or peptide residue, and R_{f} is the side-chain of a natural amino acid in which any functional group present is optionally protected;
Re, an optionally protected amino acid, azaamino acid or peptide residue, and, when W is N(R), then X, N and the substituent R on N together may form a saturated or unsaturated cyclic, bicyclic or fused ring system, or N, A′ and the substituent R on N together form a saturated or unsaturated cyclic, bicyclic, or fused ring system.

2. A method of treating Alzheimer's disease in a subject in need of such treatment comprising administering to the subject a compound disclosed in claim 1, or a pharmaceutically acceptable salt thereof.

3. A method of treating Alzheimer's disease by modulating the activity of beta amyloid converting enzyme, comprising administering to a subject in need of such treatment a compound disclosed in claim 1, or a pharmaceutically acceptable salt thereof.

4. The method according to claim 1, further comprising the administration of a P-gp inhibitor, or a pharmaceutically acceptable salt thereof.

5. A method of treating a subject who has, or in preventing a subject from getting, a disease or condition selected from the group consisting of Alzheimer's disease, for helping prevent or delay the onset of Alzheimer's disease, for treating subjects with mild cognitive impairment (MCI) and preventing or delaying the onset of Alzheimer's disease in those who would progress from MCI to AD, for treating Down'syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, or diffuse Lewy body type of Alzheimer's disease and who is in need of such treatment which includes administration of a therapeutically effective amount of a compound of formula (I), or a pharmaceutically acceptable salt thereof: 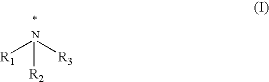
wherein R₁ is selected from R,
wherein R is selected from the group consisting of hydrogen, —R′H, —R′C(O)OR″, —R′C(O)NH₂, —R′C(O)NHR″, —R′C(O)NR″R′″, —R′NHC(O)R″, —R′NR′″C(O)R″, and —R′C(O)R″,
where R″ and R′″ are independently selected from (C₁-C₁₈)alkyl; (C₃-C₁₈)cycloalkyl; (C₃-C₁₈)cycloalkyl (C₁-C₁₈)alkyl; (C₆-C₂₄)aryl; (C₇-C₂₅)aralkyl; (C₂-C₁₈)alkenyl; (C₈-C₂₆)aralkenyl; (C₂-C₁₈)alkynyl; (C₈-C₂₆)aralkynyl; or heterocyclic; all optionally substituted,
where R′ is a divalent radical derived from (C₁-C₁₈)alkyl; (C₃-C₁₈)cycloalkyl; (C₃-C₁₈)cycloalkyl(C₁-C₁₈)alkyl; (C₆-C₂₄)aryl; (C₇-C₂₅)aralkyl; (C₂-C₁₈)alkenyl; (C₈-C₂₆)aralkenyl; (C₂-C₁₈)alkynyl; (C₈-C₂₆)aralkynyl; or heterocyclic; all optionally substituted;
and the moeity 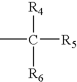
where R₄, R₅ and R₆ are independently a group R as defined above; or R₄ has the meaning of R as defined above and R₅ and R₆ taken together are ═O, ═S, ═NH or ═NR;
R₂ is 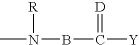
where R is as previously defined;
D is O or S;
Y is selected from hydrogen, —R or —OR, and an amino acid, aza-amino acid or peptide residue in which any functional group present is optionally protected; and
B is optionally absent or is (C₁-C₆)alkylidene, wherein any one or more —CH₂— groups may be replaced by —NR—, —NH—, —O— or —S—, provided that the compound of Formula (I) does not contain a chain of three or more atoms which are not carbon, and wherein any H atom may be substituted by a group R as previously defined;
N*, N, R₁ and R can be optionally taken together to form a cyclic diazaalkane of the formula: 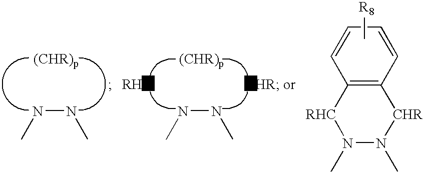
where p is 1 to 3,
each R is independently as defined above, and
R₈ is selected from R, —NH₂, —NHR, —NR₂, —COOH, —COOL, —CHO, —C(O)R, —CN, halo, —CF₃, —OL, —SR, —S(O)R, —S(O)₂R, —CONH₂, —CONHR, —CONR₂, —NHOH, —NHOL, —NO₂, ═O, ═S or —NHNH₂,
wherein each R is independently as defined above, and wherein L is independently R or a hydroxyl protecting group;
or
R₂, N* and R₄ together form a saturated or unsaturated cyclic, bicyclic or fused ring system which may be additionally substituted by —C(O)Y, where Y is as previously defined;
R₃ is X—W-A′-Q-A-, wherein: A′ and A independently are absent or (C₁-C₈)alkylidene which may be substituted with one or more substituents R as previously defined;
Q is 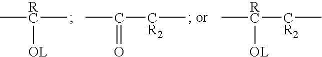
where L and each R, independently of the others, are as previously defined, and optionally Q and A together, or Q and A′ together, or A′, Q and A together form part of a saturated or unsaturated cyclic, bicyclic or fused ring system;
W is absent, or is selected from N(R), O or S,
wherein R is as previously defined; and
X is selected from hydrogen, X₁, where X₁ is Ra— or RbC(O)— or RbS(O)_{z}—,
where z is 1 or 2,
where Ra and Rb are independently (C₁-C₁₈)alkyl; (C₃-C₁₈)cycloalkyl; (C₃-C₁₈)cycloalkyl (C₁-C₁₈)alkyl; heterocyclic; (C₁-C₁₈) alkylheterocyclic; heterocyclic(C₆-C₂₄)aryloxy; (C₁-C₁₈)alkoxy; (C₁-C₁₈)alkoxy(C₁-C₁₈)alkyl; (C₁-C₁₂)alkyl; (C₆-C₂₄)aryloxy(C₁-C₁₈)alkyl; (C₆-C₂₄)aryloxy(C₁-C₁₈)alkoxy; (C₆-C₂₄)aryl; (C₆-C₂₄)aryl(C₁-C₁₈)alkyl; (C₆-C₂₄)aryl(C₁-C₁₈)alkylheterocyclic; (C₁-C₁₂)alkylheterocyclic; heterocyclicoxy(C₁-C,₈) alkyl; (C₁-C18) alkylamino; di(C₁-C₁₈)alkylamino; (C₆-C₂₄)arylamino; di(C₆-C₂₄)arylamino; (C₇-C₂₅) aralkylamino; or di(C₇-C₂₅)aralkylamino; any of which may be optionally substituted with one or more groups selected from —F, —Cl, —Br, —I, —CF₃, —OH, —OR_{IV}, —NH₂, —NHR_{IV}, —NR_{IV}R_{V}, —CN, —NO₂, —SH, —SR_{IV}, —SOR_{IV}, —SO₂R_{IV}, ═O, ═S, ═NOH, ═NOR_{IV}, —NHOH, —NHOR_{IV}, —CHO, where R_{IV} and R_{V} are independently (C₁-C₁₈)alkyl; (C₃-C₁₈)cycloalkyl; (C₃-C₁₈)-cycloalkyl (C₁-C₁₈)alkyl; (C₆-C₂₄)-aryl; (C₇-C₂₅)aralkyl; (C₂-C₁₈)alkenyl; (C₈-C₂₆)aralkenyl; (C₂-C₁₈)alkynyl; (C₈-C₂₆)-aralkynyl; or heterocyclic; and Re,
where Re is a group of the formula: 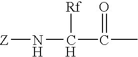
where Z has the meaning of Ra or Rb or is an acylated amino acid, azaamino acid or peptide residue, and R_{f} is the side-chain of a natural amino acid in which any functional group present is optionally protected;
Re, an optionally protected amino acid, azaamino acid or peptide residue, and, when W is N(R), then X, N and the substituent R on N together may form a saturated or unsaturated cyclic, bicyclic or fused ring system, or N, A′ and the substituent R on N together form a saturated or unsaturated cyclic, bicyclic, or fused ring.

6. The method according to claim 5 wherein the compound of formula (I) is selected from the group consisting of:
(i) t-butyl 3-isopropyl-3-[(2R or S,3S)-2-hydroxy-3-(phenylmethoxycarbonyl)amino-4-phenylbutyl]carbazate,
(ii) t-butyl 3-isopropyl-3-[(2R or S,3S)-2-hydroxy-3-(N-quinaldoyl-L-valyl)amino-4-phenylbutyl]carbazate,
(iii) t-butyl 3-isopropyl-3-[(2R or S,3S)-2-hydroxy-3-(N-quinaldoyl-L-asparaginyl)amino-4-phenylbutyl]carbazate,
(iv) t-butyl 3-isopropyl-3- [(3S) -2-oxo-3- (N-quinaldoyl-L-asparaginyl)amino-4-phenylbuty1]carbazate,
(v) t-butyl 3-(1-methyl-3-phenylpropen-3-yl)-3-[(2R or S,3S)-2-hydroxy-3-(phenylmethoxycarbonyl)amino-4-phenylbutyl]carbazate,
(vi) t-butyl 3-(1-methyl-3-phenylpropyl)-3-[(2R or S,3S)-2-hydroxy-3-(N-quinaldoyl-L-asparaginyl)amino-4-phenylbutyl]carbazate,
(vii) cis-1,6-3-t-butoxycarbonyl-4-[(2R or S,3S)-2-hydroxy-3-amino-4-phenylbutyl]-3,4-diazabicyclo[4.4.0]decane,
(viii) cis-1,6-3-t-butoxycarbonyl-4-[(2R or S,3S)-2-hydroxy-3-(phenylmethoxycarbonyl)amino-4-phenylbutyl]-diazabicyclo[4.4.0]decane,
(ix) cis-1,6-3-t-butoxycarbonyl-4-[(2R or S,3S)-2-hydroxy-3-(N-quinaldoyl-L-valyl)amino-4-phenylbutyl]-3,4-diazabicyclo[4.4.0]decane
(x) cis-1,6-3-t-butoxycarbonyl-4-[(2R or S,3S)-2-hydroxy-3-[N-(2-pyridyl)methoxycarbonyl)-L-valyl)amino-4-phenylbutyl]-3,4-diazabicyclo[4:4.0]decane
(xi) cis-1,6-3-t-butoxycarbonyl-4-[(2R or S,3S)-2-hydroxy-3-(N-quinaldoyl-L-asparaginyl)amino-4-phenylbutyl]-3,4-diazabicyclo[4.4.0]decane,
(xii) cis-1,6-3-t-butoxycarbonyl-4-[(2R or S,3S)-2-hydroxy-3-(N-quinaldoyl-glutaminyl)amino-4-phenylbutyl]-3,4odiazabicyclo[4.4.0]decane,
(xiii) cis-1,6-3-t-butoxycarbonyl-4-[(2R or S,3S)-2-hydroxy-3-(N-quinaldoyl-L-threonyl)amino-4-phenylbutyl]-3,4-diazabicyclo[4.4.0]decane,
(xiv) 2-t-butoxycarbonyl-3-[(2R or S,3S)-2-hydroxy-3-(phenylmethoxycarbonyl)amino-4-phenylbutyl]-2,3-diazabicyclo[2.2.1]hept-5-ene,
(xv) 2-t-butoxycarbonyl-3-[(2R or S,3S)-2-hydroxy-3-phenylmethoxycarbonyl)amino-4-phenylbutyl]-2,3-diaza-bicyclo[2.2.1]heptane,
(xvi) 2-t-butoxycarbonyl-3-[(2R or S,3S)-2-hydroxy-3-(N-(2-pyridyl)methoxy-L-valyl)amino-4-phenylbutyl]-2,3-diaza-bicyclo[2.2.1 ]heptane,
(xvii) 2-[N-(1S)(2-methyl-1-methoxycarbonylpropyl) carbamoyl]-3-[(2R or S,3S)-2-hydroxy-3-[N-(2-pyridyl)methoxy-L-valyl]amino-4-phenylbutyl]-2,3-diazabicyclo[2.2.1]heptane,
(xviii) 2-t-butoxycarbonyl-3-[(2R or S,3S)-2-hydroxy-3-(N-quinaldoyl-L-asparaginyl)amino-4-phenylbutyl]-2,3-diazabicyclo[2.2.1]heptane,
(ixx) 1-[2-(2-pyridyl)methoxycarbonylamino-]benzoyl-2-[(2R or S,3S)-2-hydroxy-3-(N-quinaldoyl-L-asparaginyl)amino-4-phenylbutyl]-2-isopropylhydrazine,
(xx) 2-t-butoxycarbonyl-3-[(2R or S,3S)-2-hydroxy-3-(N-quinaldoyl-L-asparaginyl)amino-4-phenylbutyl]-1,2,3,4-tetrahydrophthalazine,
(xxi) 1-trimethylacetyl-2-[(2R or S,3S)-2-hydroxy-3-(phenylmethoxycarbonyl)amino-4-phyenylbutyl]-2-isopropyl hydrazine,
(xxii) 1-trimethylacetyl-2-[(2R or S,3S)-2-hydroxy-3-(N-quinaldoyl-L-asparaginyl)amino-4-phenylbutyl]-2-isoprolaylhydrazine,
(xxiii) 1-(t-butylamino)carbonyl-2-[(2R or S,3S)-2-hydroxy-3-(N-quinaldoyl-L-asparaginyl)amino-4-phenylbutyl]-2-isopropylhydrazine,
(xxiv) t-butyl 3-isopropyl-3-[(2R or S,38)-2-hydroxy-3-(N-picolinoyl-L-asparaginyl)amino-4-phenylbutyl]carbazate,
(xxv) t-butyl 3-isopropyl-3-[(2R or S,3S)-2-hydroxy-3-(N-(2-pyridyl)methoxycarbonyl-anthraniloyl)amino-4-phenylbutyl]carbazate.
(xxvi) t-butyl 3-benzyl-3-[(2R or S,3S)-2-hydroxy-3-(phenylmethoxycarbonyl)amino-4-phenylbutyl]carbazate,
(xxvii) t-butyl 3-benzyl-3-[(2R or S,3S)-2-hydroxy-3-(N-quinaldoyl-L-asparaginyl)amino-4-phenylbutyl]carbazate,
(xxviii) t-butyl 3-cyclohexyl-3-[(2R or S, 3S)-2-hydroxy-3-(phenyl-methoxycarbonyl)amino-4-phenylbutyl]carbazate,
(xxix) t-butyl 3-cyclohexyl-3-[(2R or S,3S)-2-hydroxy-3-(N-quinaldoyl-L-asparaginyl)amino-4-phenylbutyl]carbazate,
(xxx) t-butyl 3-isopropyl-3-[(2R or S,3S)-2-hydroxy-3-(N-(1-carbamoylmethyl)acryloyl)amino-4-phenylbutyl]carbazate,
(xxxi) t-butyl 3-isopropyl-3-[(2R or S,3S)-2-hydroxy-3-(N-(2(RS)-3-tert-butylthio-2-carbamoyl-methylpropionyl)amino-4-phenylbutyl]carbazate,
(xxxii) t-butyl 3-isopropyl-3-[(2R or S,3S)-2-hydroxy-3-(N-(1-benzoyl-L-asparaginyl)amino-4-phenylbutyl]carbazate,
(xxxiii) 1-t-butyloxycarbonyl-2-[(2R or S,3S)-2-hydroxy-3-(phenylmethoxycarbonyl)amino-4-phenylbutyl]hexahydropyridazine,
(xxxiv) 1-t-butyloxycarbonyl-2-[(2R or S,3S)-2-hydroxy-3-(N-quinaldoyl-L-asparaginyl)amino-4-phenylbutyl]hexahydropyridazine,
(xxxv) cis-1,6-3-t-butoxycarbonyl-4-[(2R or S, 3S)-2-hydroxy-3-(N-quinaldoyl-3-cyano-L-alanyl)amino-4-phenylbutyl]-3,4-diazabicyclo[4,4,0]decane;
or pharmaceutically acceptable salts thereof.

7. A method of treating or preventing Alzheimer's disease in a subject in need of such treatment comprising administering a therapeutically effective amount of a composition comprising one or more pharmaceutically acceptable carriers and a compound of Formula (I) or a pharmaceutically acceptable salt thereof: 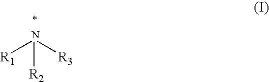
wherein R₁ is selected from R,
wherein R is selected from the group consisting of hydrogen, —R′H, —R′C(O)OR″, —R′C(O)NH₂, —R′C(O)NHR″, —R′C(O)NR″R′″, —R′NHC(O)R″, —R′NR′″C(O)R″, and —R′C(O)R″,
where R″ and R′″ are independently selected from (C₁-C₁₈)alkyl; (C₃-C₁₈)cycloalkyl; (C₃-C₁₈) cycloalkyl(C₁-C₁₈)alkyl; (C₆-C₂₄)aryl; (C₇-C₂₅)aralkyl; (C₂-C₁₈)alkenyl; (C₈-C₂₆)aralkenyl; (C₂-C₁₈)alkynyl; (C₈-C₂₆)aralkynyl; or heterocyclic; all optionally substituted,
where R′ is a divalent radical derived from (C₁-C₁₈)alkyl; (C₃-C₁₈)cycloalkyl; (C₃-C₁₈)cycloalkyl(C₁-C₁₈)alkyl; (C₆-C₂₄)aryl; (C₇-C₂₅)aralkyl; (C₂-C₁₈)alkenyl; (C₈-C₂₆)aralkenyl; (C₂-C₁₈)alkynyl; (C₈-C₂₆)aralkynyl; or heterocyclic; all optionally substituted;
and the moeity 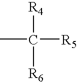
where R₄, R₅ and R₆ are independently a group R as defined above; or R₄ has the meaning of R as defined above and R₅ and R₆ taken together are ═O, ═S, ═NH or ═NR;
R₂ is 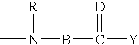
where R is as previously defined;
D is O or S;
Y is selected from hydrogen, —R or —OR, and an amino acid, aza-amino acid or peptide residue in which any functional group present is optionally protected; and
B is optionally absent or is (C₁-C₆)alkylidene, wherein any one or more —CH₂— groups may be replaced by —NR—, —NH—, —O— or —S—, provided that the compound of Formula (I) does not contain a chain of three or more atoms which are not carbon, and wherein any H atom may be substituted by a group R as previously defined;
N*, N, R₁ and R can be optionally taken together to form a cyclic diazaalkane of the formula: 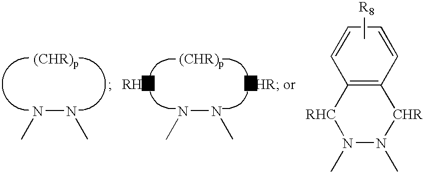
where p is 1 to 3,
each R is independently as defined above, and R₈ is selected from R, —NH₂, —NHR, —NR₂, —COOH, —COOL, —CHO, —C(O)R, —CN, halo, —CF₃, —OL, —SR, —S(O)R, —S(O)₂R, —CONH₂, —CONHR, —CONR₂, —NHOH, —NHOL, —NO₂, ═O, ═S or —NHNH₂,
wherein each R is independently as defined above, and wherein L is independently R or a hydroxyl protecting group;
or
R₂, N* and R₄ together form a saturated or unsaturated cyclic, bicyclic or fused ring system which may be additionally substituted by —C(O)Y, where Y is as previously defined;
R₃ is X—W-A′-Q-A-, wherein: A′ and A independently are absent or (C₁-C₈)alkylidene which may be substituted with one or more substituents R as previously defined;
Q is 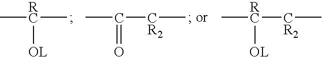
where L and each R, independently of the others, are as previously defined, and optionally Q and A together, or Q and A′ together, or A′, Q and A together form part of a saturated or unsaturated cyclic, bicyclic or fused ring system;
W is absent, or is selected from N(R), O or S,
wherein R is as previously defined; and
X is selected from hydrogen, X₁, where X₁ is Ra— or RbC(O)— or RbS(O)_{z}—,
where z is 1 or 2,
where Ra and Rb are independently (C₁-C₁₈)alkyl; (C3-C₁₈)cycloalkyl; (C₃-C₁₈)cycloalkyl(C₁-C₁₈)alkyl; heterocyclic; (C₁-C₁₈)alkylheterocyclic; heterocyclic(C₆-C₂₄)aryloxy; (C₁-C₁₈)alkoxy; (C₁-C₁₈)alkoxy(C₁-C₁₈)alkyl; (C₁-C₁₂)alkyl; (C₆-C₂₄)aryloxy(C₁-C₁₈)alkyl; (C₆-C₂₄)aryloxy(C₁-C₁₈)alkoxy; (C₆-C₂₄)aryl; (C₆-C₂₄)aryl(C₁-C₁₈)alkyl; (C₆-C₂₄) aryl(C₁-C₁₈)alkylheterocyclic; (C₁-C₁₂)alkylheterocyclic; heterocyclicoxy(C₁-C₁₈)alkyl; (C₁-C₁₈)alkylamino; di(C₁-C₁₈)alkylamino; (C₆-C₂₄)arylamino; di(C₆-C₂₄)arylamino; (C₇-C₂₅)aralkylamino; or di(C₇-C₂₅)aralkylamino; any of which may be optionally substituted with one or more groups selected from —F, —Cl, —Br, —I, —CF₃, —OH, —OR_{IV}, —NH₂, —NHR_{IV}, —NR_{IV}R_{V}, —CN, —NO₂, —SH, —SR_{IV}, —SOR_{IV}, —SO₂R_{IV}, ═O, ═S, ═NOH, ═NOR_{IV}, —NHOH, —NHOR_{IV}, —CHO, where R_{IV} and R_{V} are independently (C₁-C₁₈)alkyl; (C₃-C₁₈)cycloalkyl; (C₃-C₁₈)-cycloalkyl(C₁-C₁₈) alkyl; (C₆-C₂₄)-aryl; (C₇-C₂₅)aralkyl; (C₂-C₁₈)alkenyl; (C₈-C₂₆)aralkenyl; (C₂-C₁₈)alkynyl; (C₈-C₂₆)-aralkynyl; or heterocyclic; and Re,
where Re is a group of the formula: 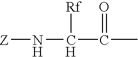
where Z has the meaning of Ra or Rb or is an acylated amino acid, azaamino acid or peptide residue, and R_{f} is the side-chain of a natural amino acid in which any functional group present is optionally protected;
Re, an optionally protected amino acid, azaamino acid or peptide residue, and, when W is N(R), then X, N and the substituent R on N together may form a saturated or unsaturated cyclic, bicyclic or fused ring system, or N, A′ and the substituent R on N together form a saturated or unsaturated cyclic, bicyclic, or fused ring.

8. (Canceled)

9. A method for inhibiting beta-secretase activity, comprising contacting an effective amount for inhibition of a compound of formula (I): 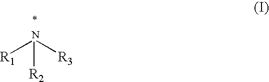
wherein R₁ is selected from R,
wherein R is selected from the group consisting of hydrogen, —R′H, —R′C(O)OR″, —R′C(O)NH₂, —R′C(O)NHR″, —R′C(O)NR″R′″, —R′NHC(O)R″, —R′NR′″C(O)R″, and —R′C(O)R″,
where R″ and R′″ are independently selected from (C₁-C₁₈)alkyl; (C₃-C₁₈)cycloalkyl; (C₃-C₁₈)cycloalkyl (C₁-C₁₈)alkyl; (C₆-C₂₄)aryl; (C₇-C₂₅)aralkyl; (C₂-C₁₈) alkenyl; (C₈-C₂₆)aralkenyl; (C₂-C₁₈)alkynyl; (C₈-C₂₆)aralkynyl; or heterocyclic; all optionally substituted,
where R′ is a divalent radical derived from (C₁-C₁₈)alkyl; (C₃-C₁₈)cycloalkyl; (C₃-C₁₈)cycloalkyl (C₁-C₁₈)alkyl; (C₆-C₂₄)aryl; (C₇-C₂₅)aralkyl; (C₂-C₁₈)alkenyl; (C₈-C₂₆)aralkenyl; (C₂-C₁₈)alkynyl; (C₈-C₂₆)aralkynyl; or heterocyclic; all optionally substituted;
and the moeity 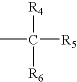
where R₄, R₅ and R₆ are independently a group R as defined above; or R₄ has the meaning of R as defined above and R₅ and R₆ taken together are ═O, ═S, ═NH or ═NR;
R₂ is 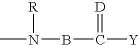
where R is as previously defined;
D is O or S;
Y is selected from hydrogen, —R or —OR, and an amino acid, aza-amino acid or peptide residue in which any functional group present is optionally protected; and
B is optionally absent or is (C₁-C₆)alkylidene, wherein any one or more —CH₂— groups may be replaced by —NR—, —NH—, —O— or —S—, provided that the compound of Formula (I) does not contain a chain of three or more atoms which are not carbon, and wherein any H atom may be substituted by a group R as previously defined;
N*, N, R₁ and R can be optionally taken together to form a cyclic diazaalkane of the formula: 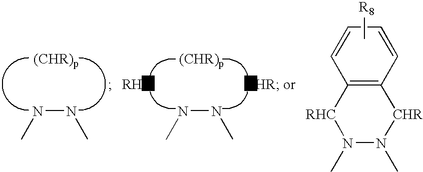
where p is 1 to 3,
each R is independently as defined above, and
R₈ is selected from R, —NH₂, —NHR, —NR₂, —COOH, —COOL, —CHO, —C(O)R, —CN, halo, —CF₃, —OL, —SR, —S(O)R, —S(O)₂R, —CONH₂, —CONHR, —CONR₂, —NHOH, —NHOL, —NO₂, ═O, ═S or —NHNH₂,
wherein each R is independently as defined above, and wherein L is independently R or a hydroxyl protecting group;
or
R₂, N* and R₄ together form a saturated or unsaturated cyclic, bicyclic or fused ring system which may be additionally substituted by —C(O)Y, where Y is as previously defined;
R₃ is X—W-A′-Q-A-, wherein: A′ and A independently are absent or (C₁-C₈)alkylidene which may be substituted with one or more substituents R as previously defined;
Q is 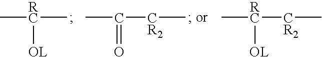
where L and each R, independently of the others, are as previously defined, and optionally Q and A together, or Q and A′ together, or A′, Q and A together form part of a saturated or unsaturated cyclic, bicyclic or fused ring system;
W is absent, or is selected from N(R), O or S,
wherein R is as previously defined; and
X is selected from hydrogen, X₁, where X₁ is Ra— or RbC(O)— or RbS(O)_{z}—,
where z is 1 or 2,
where Ra and Rb are independently (C₁-C₁₈)alkyl; (C₃-C₁₈)cycloalkyl; (C₃-C₁₈)cycloalkyl (C₁-C₁₈)alkyl; heterocyclic; (C₁-C₁₈) alkylheterocyclic; heterocyclic(C₆-C₂₄)aryloxy; (C₁-C₁₈)alkoxy; (C₁-C₁₈)alkoxy(C₁-C₁₈)alkyl; (C₁-C₁₂)alkyl; (C₆-C₂₄)aryloxy(C₁-C₁₈)alkyl; (C₆-C₂₄)aryloxy(C₁-C₁₈)alkoxy; (C₆-C₂₄)aryl; (C₆-C₂₄)aryl(C₁-C₁₈)alkyl; (C₆-C₂₄) aryl(C₁-C₁₈)alkylheterocyclic; (C₁-C₁₂)alkylheterocyclic; heterocyclicoxy(C₁-C₁₈)alkyl; (C₁-C₁₈)alkylamino; di (C₁-C₁₈)alkylamino; (C₆-C₂₄)arylamino; di(C₆-C₂₄)arylamino; (C₇-C₂₅)aralkylamino; or di(C₇-C₂₅)aralkylamino; any of which may be optionally substituted with one or more groups selected from —F, —Cl, —Br, —I, —CF₃, —OH, —OR_{IV}, —NH₂, —NHR_{IV}, —NR_{IV}R_{V}, —CN, —NO₂, —SH, —SR_{IV}, —SOR_{IV}, —SO₂R_{IV}, ═O, ═S, ═NOH, ═NOR_{IV}, —NHOH, —NHOR_{IV}, —CHO, where R_{IV} and R_{V} are independently (C₁-C₁₈)alkyl; (C₃-C₁₈)cycloalkyl; (C₃-C₁₈)-cycloalkyl(C₁-C₁₈)alkyl; (C₆-C₂₄)-aryl; (C₇-C₂₅)aralkyl; (C₂-C₁₈)alkenyl; (C₈-C₂₆)aralkenyl; (C₂-C₁₈)alkynyl; (C₈-C₂₆)-aralkynyl; or heterocyclic; and Re,
where Re is a group of the formula: 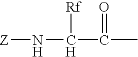
where Z has the meaning of Ra or Rb or is an acylated amino acid, azaamino acid or peptide residue, and R_{f} is the side-chain of a natural amino acid in which any functional group present is optionally protected;
Re, an optionally protected amino acid, azaamino acid or peptide residue, and, when W is N(R), then X, N and the substituent R on N together may form a saturated or unsaturated cyclic, bicyclic or fused ring system, or N, A′ and the substituent R on N together form a saturated or unsaturated cyclic, bicyclic, or fused ring.

10. A method for inhibiting cleavage of an amyloid precursor protein (APP) isotype at a site in the APP isotype that is susceptible to cleavage, comprising contacting said APP isotype with an effective cleavage inhibitory amount of a compound of formula (I): 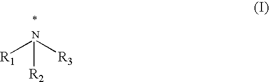
wherein R₁ is selected from R,
wherein R is selected from the group consisting of hydrogen, —R′H, —R′C(O)OR″, —R′C(O)NH₂, —R′C(O)NHR″, —R′C(O)NR″R′″, —R′NHC(O)R″, —R′NR′″C(O)R″, and —R′C(O)R″,
where R″ and R′″ are independently selected from (C₁-C₁₈)alkyl; (C₃-C₁₈)cycloalkyl; (C₃-C₁₈)cycloalkyl(C₁-C₁₈) alkyl; (C₆-C₂₄)aryl; (C₇-C₂₅)aralkyl; (C₂-C₁₈)alkenyl; (C₈-C₂₆)aralkenyl; (C₂-C₁₈)alkynyl; (C₈-C₂₆)aralkynyl; or heterocyclic; all optionally substituted,
where R′ is a divalent radical derived from (C₁-C₁₈)alkyl; (C₃-C₁₈)cycloalkyl; (C₃-C₁₈)cycloalkyl(C₁-C₁₈)alkyl; (C₆-C₂₄)aryl; (C₇-C₂₅)aralkyl; (C₂-C₁₈)alkenyl; (C₈-C₂₆)aralkenyl; (C₂-C₁₈)alkynyl; (C₈-C₂₆)aralkynyl; or heterocyclic; all optionally substituted;
and the moeity 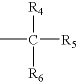
where R₄, R₅ and R₆ are independently a group R as defined above; or R₄ has the meaning of R as defined above and R₅ and R₆ taken together are ═O, ═S, ═NH or ═NR;
R₂ is 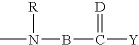
where R is as previously defined;
D is O or S;
Y is selected from hydrogen, —R or —OR, and an amino acid, aza-amino acid or peptide residue in which any functional group present is optionally protected; and
B is optionally absent or is (C₁-C₆)alkylidene, wherein any one or more —CH₂— groups may be replaced by —NR—, —NH—, —O— or —S—, provided that the compound of Formula (I) does not contain a chain of three or more atoms which are not carbon, and wherein any H atom may be substituted by a group R as previously defined;
N*, N, R₁ and R can be optionally taken together to form a cyclic diazaalkane of the formula: 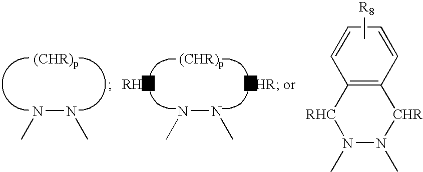
where p is 1 to 3,
each R is independently as defined above, and
R₈ is selected from R, —NH₂, —NHR, —NR₂, —COOH, —COOL, —CHO, —C(O)R, —CN, halo, —CF₃, —OL, —SR, —S(O)R, —S(O)₂R, —CONH₂, —CONHR, —CONR₂, —NHOH, —NHOL, —NO₂, ═O, ═S or —NHNH₂,
wherein each R is independently as defined above,
and wherein L is independently R or a hydroxyl protecting group;
or
R₂, N* and R₄ together form a saturated or unsaturated cyclic, bicyclic or fused ring system which may be additionally substituted by —C(O)Y, where Y is as previously defined;
R₃ is X—W-A′-Q-A-, wherein: A′ and A independently are absent or (C₁-C₈)alkylidene which may be substituted with one or more substituents R as previously defined;
Q is 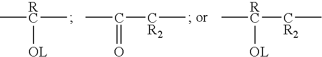
where L and each R, independently of the others, are as previously defined, and optionally Q and A together, or Q and A′ together, or A′, Q and A together form part of a saturated or unsaturated cyclic, bicyclic or fused ring system;
W is absent, or is selected from N(R), O or S,
wherein R is as previously defined; and
X is selected from hydrogen, X₁, where X₁ is Ra— or RbC(O)— or RbS(O)_{z}—,
where z is 1 or 2,
where Ra and Rb are independently (C₁-C₁₈)alkyl; (C₃-C₁₈)cycloalkyl; (C₃-C₁₈)cycloalkyl(C₁-C₁₈)alkyl; heterocyclic; (C₁-C₁₈)alkylheterocyclic; heterocyclic(C₆-C₂₄)aryloxy; (C₁-C₁₈)alkoxy; (C₁-C₁₈)alkoxy(C₁-C₁₈)alkyl; (C₁-C₁₂)alkyl; (C₆-C₂₄)aryloxy(C₁-C₁₈)alkyl; (C₆-C₂₄)aryloxy(C₁-C₁₈)alkoxy; (C₆-C₂₄)aryl; (C₆-C₂₄)aryl(C₁-C₁₈)alkyl; (C₆-C₂₄)aryl(C₁-C₁₈)alkylheterocyclic; (C₁-C₁₂)alkylheterocyclic; heterocyclicoxy(C₁-C₁₈)alkyl; (C₁-C₁₈)alkylamino; di(C₁-C₁₈)alkylamino; (C₆-C₂₄)arylamino; di(C₆-C₂₄)arylamino; (C₇-C₂₅)aralkylamino; or di(C₇-C₂₅)aralkylamino; any of which may be optionally substituted with one or more groups selected from —F, —Cl, —Br, —I, —CF₃, —OH, —OR_{IV}, —NH₂, —NHR_{IV}, —NR_{IV}R_{V}, —CN, —NO₂, —SH, —SR_{IV}, —SOR_{IV}, —SO₂R_{IV}, ═O, ═S, ═NOH, ═NOR_{IV}, —NHOH, —NHOR_{IV}, —CHO, where R_{IV} and R_{V} are independently (C₁-C₁₈)alkyl; (C₃-C₁₈)cycloalkyl; (C₃-C₁₈)-cycloalkyl(C₁-C₁₈)alkyl; (C₆-C₂₄)-aryl; (C₇-C₂₅)aralkyl; (C₂-C₁₈)alkenyl; (C₈-C₂₆)aralkenyl; (C₂-C₁₈)alkynyl; (C₈-C₂₆)-aralkynyl; or heterocyclic; and Re,
where Re is a group of the formula: 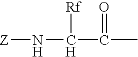
where Z has the meaning of Ra or Rb or is an acylated amino acid, azaamino acid or peptide residue, and R_{f} is the side-chain of a natural amino acid in which any functional group present is optionally protected;
Re, an optionally protected amino acid, azaamino acid or peptide residue, and, when W is N(R), then X, N and the substituent R on N together may form a saturated or unsaturated cyclic, bicyclic or fused ring system, or N, A′ and the substituent R on N together form a saturated or unsaturated cyclic, bicyclic, or fused ring.

11. A method for inhibiting production of amyloid beta peptide (A beta) in a cell, comprising administering to said cell an effective inhibitory amount of a compound of formula (I): 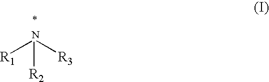
wherein R₁ is selected from R,
wherein R is selected from the group consisting of hydrogen, —R′H, —R′C(O)OR″, —R′C(O)NH₂, —R′C(O)NHR″, —R′C(O)NR″R′″, —R′NHC(O)R″, —R′NR′″C(O)R″, and —R′C(O)R″,
where R″ and R′″ are independently selected from (C₁-C₁₈)alkyl; (C₃-C₁₈)cycloalkyl; (C₃-C₁₈)cycloalkyl(C₁-C₁₈)alkyl; (C₆-C₂₄)aryl; (C₇-C₂₅)aralkyl; (C₂-C₁₈)alkenyl; (C₈-C₂₆)aralkenyl; (C₂-C₁₈)alkynyl; (C₈-C₂₆)aralkynyl; or heterocyclic; all optionally substituted,
where R′ is a divalent radical derived from (C₁-C₁₈)alkyl; (C₃-C₁₈)cycloalkyl; (C₃-C₁₈)cycloalkyl(Cₗ-C₁₈)alkyl; (C₆-C₂₄)aryl; (C₇-C₂₅)aralkyl; (C₂-C₁₈)alkenyl; (C₈-C₂₆)aralkenyl; (C₂-C₁₈)alkynyl; (C₈-C₂₆)aralkynyl; or heterocyclic; all optionally substituted;
and the moeity 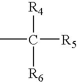
where R₄, R₅ and R₆ are independently a group R as defined above; or R₄ has the meaning of R as defined above and R₅ and R₆ taken together are ═O, ═S, ═NH or ═NR;
R₂ is 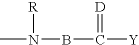
where R is as previously defined;
D is O or S;
Y is selected from hydrogen, —R or —OR, and an amino acid, aza-amino acid or peptide residue in which any functional group present is optionally protected; and
B is optionally absent or is (C₁-C₆)alkylidene, wherein any one or more —CH₂— groups may be replaced by —NR—, —NH—, —O— or —S—, provided that the compound of Formula (I) does not contain a chain of three or more atoms which are not carbon, and wherein any H atom may be substituted by a group R as previously defined;
N*, N, R₁ and R can be optionally taken together to form a cyclic diazaalkane of the formula: 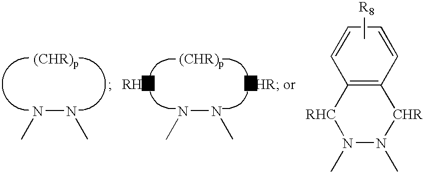
where p is 1 to 3,
each R is independently as defined above, and
R₈ is selected from R, —NH₂, —NHR, —NR₂, —COOH, —COOL, —CHO, —C(O)R, —CN, halo, —CF₃, —OL, —SR, —S(O)R, —S(O)₂R, —CONH₂, —CONHR, —CONR₂, —NHOH, —NHOL, —NO₂, ═O, ═S or —NHNH₂,
wherein each R is independently as defined above, and wherein L is independently R or a hydroxyl protecting group;
or
R₂, N* and R₄ together form a saturated or unsaturated cyclic, bicyclic or fused ring system which may be additionally substituted by —C(O)Y, where Y is as previously defined;
R₃ is X—W-A′-Q-A-, wherein: A′ and A independently are absent or (C₁-C₈)alkylidene which may be substituted with one or more substituents R as previously defined;
Q is 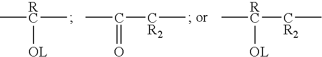
where L and each R, independently of the others, are as previously defined, and optionally Q and A together, or Q and A′ together, or A′, Q and A together form part of a saturated or unsaturated cyclic, bicyclic or fused ring system;
W is absent, or is selected from N(R), O or S,
wherein R is as previously defined; and
X is selected from hydrogen, X₁, where X₁ is Ra— or RbC(O)— or RbS(O)_{z}—,
where z is 1 or 2,
where Ra and Rb are independently (C₁-C₁₈)alkyl; (C₃-C₁₈)cycloalkyl; (C₃-C₁₈)cycloalkyl(C₁-C₁₈)alkyl; heterocyclic; (C₁-C₁₈)alkylheterocyclic; heterocyclic(C₆-C₂₄)aryloxy; (C₁-C₁₈)alkoxy; (C₁-C₁₈)alkoxy(C₁-C₁₈)alkyl; (C₁-C₁₂)alkyl; (C₆-C₂₄)aryloxy(C₁-C₁₈)alkyl; (C₆-C₂₄)aryloxy(C₁-C₁₈) alkoxy; (C₆-C₂₄)aryl; (C₆-C₂₄)aryl(C₁-C₁₈)alkyl; (C₆-C₂₄)aryl(C₁-C₁₈)alkylheterocyclic; (C₁-C₁₂)alkylheterocyclic; heterocyclicoxy(C₁-C₁₈)alkyl; (C₁-C₁₈)alkylamino; di(C₁-C₁₈)alkylamino; (C₆-C₂₄)arylamino; di(C₆-C₂₄)arylamino; (C₇-C₂₅)aralkylamino; or di(C₇-C₂₅)aralkylamino; any of which may be optionally substituted with one or more groups selected from —F, —Cl, —Br, —I, —CF₃, —OH, —OR_{IV}, —NH₂, —NHR_{IV}, —NR_{IV}R_{V}, —CN, —NO₂, —SH, —SR_{IV}, —SOR_{IV}, —SO₂R_{IV}, ═O, ═S, ═NOH, ═NOR_{IV}, —NHOH, —NHOR_{IV}, —CHO, where R_{IV} and R_{V} are independently (C₁-C₁₈)alkyl; (C₃-C₁₈)cycloalkyl; (C₃-C₁₈)-cycloalkyl(C₁-C₁₈)alkyl; (C₆-C₂₄)-aryl; (C₇-C₂₅)aralkyl; (C₂-C₁₈)alkenyl; (C₈-C₂₆)aralkenyl; (C₂-C₁₈)alkynyl; (C₈-C₂₆)-aralkynyl; or heterocyclic; and Re,
where Re is a group of the formula: 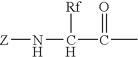
where Z has the meaning of Ra or Rb or is an acylated amino acid, azaamino acid or peptide residue, and R_{f} is the side-chain of a natural amino acid in which any functional group present is optionally protected;
Re, an optionally protected amino acid, azaamino acid or peptide residue, and, when W is N(R), then X, N and the substituent R on N together may form a saturated or unsaturated cyclic, bicyclic or fused ring system, or N, A′ and the substituent R on N together form a saturated or unsaturated cyclic, bicyclic, or fused ring.

12. The method of claim 11, wherein the cell is an animal cell.

13. The method of claim 12, wherein the animal cell is a mammalian cell.

14. The method of claim 13, wherein the mammalian cell is human.

15. A composition comprising beta-secretase complexed with a compound of formula (I): 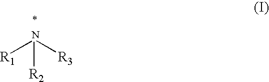
wherein R₁ is selected from R,
wherein R is selected from the group consisting of hydrogen, —R′H, —R′C(O)OR″, —R′C(O)NH₂, —R′C(O)NHR″, —R′C(O)NR″R′″, —R′NHC(O)R″, —R′NR′″C(O)R″, and —R′C(O)R″,
where R″ and R′″ are independently selected from (C₁-C₁₈)alkyl; (C₃-C₁₈)cycloalkyl; (C₃-C₁₈)cycloalkyl(C₁-C₁₈)alkyl; (C₆-C₂₄)aryl; (C₇-C₂₅)aralkyl; (C₂-C₁₈)alkenyl; (C₈-C₂₆)aralkenyl; (C₂-C₁₈)alkynyl; (C₈-C₂₆)aralkynyl; or heterocyclic; all optionally substituted,
where R′ is a divalent radical derived from (C₁-C₁₈)alkyl; (C₃-C₁₈)cycloalkyl; (C₃-C₁₈)cycloalkyl(C₁-C₁₈)alkyl; (C₆-C₂₄)aryl; (C₇-C₂₅)aralkyl; (C₂-C₁₈)alkenyl; (C₈-C₂₆)aralkenyl; (C₂-C₁₈)alkynyl; (C₈-C₂₆)aralkynyl; or heterocyclic; all optionally substituted;
and the moeity 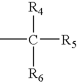
where R₄, R₅ and R₆ are independently a group R as defined above; or R₄ has the meaning of R as defined above and R₅ and R₆ taken together are ═O, ═S, ═NH or ═NR;
R₂ is 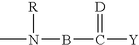
where R is as previously defined;
D is O or S;
Y is selected from hydrogen, —R or —OR, and an amino acid, aza-amino acid or peptide residue in which any functional group present is optionally protected; and
B is optionally absent or is (C₁-C₆)alkylidene, wherein any one or more —CH₂— groups may be replaced by —NR—, —NH—, —O— or —S—, provided that the compound of Formula (I) does not contain a chain of three or more atoms which are not carbon, and wherein any H atom may be substituted by a group R as previously defined;
N*, N, R₁ and R can be optionally taken together to form a cyclic diazaalkane of the formula: 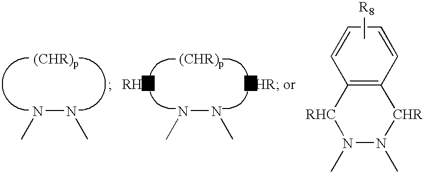
where p is 1 to 3,
each R is independently as defined above, and
R₈ is selected from R, —NH₂, —NHR, —NR₂, —COOH, —COOL, —CHO, —C(O)R, —CN, halo, —CF₃, —OL, —SR, —S(O)R, —S(O)₂R, —CONH₂, —CONHR, —CONR₂, —NHOH, —NHOL, —NO₂, ═O, ═S or —NHNH₂,
wherein each R is independently as defined above, and wherein L is independently R or a hydroxyl protecting group;
or
R₂, N* and R₄ together form a saturated or unsaturated cyclic, bicyclic or fused ring system which may be additionally substituted by —C(O)Y, where Y is as previously defined;
R₃ is X—W-A′-Q-A-, wherein: A′ and A independently are absent or (C₁-C₈)alkylidene which may be substituted with one or more substituents R as previously defined;
Q is 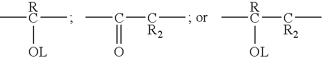
where L and each R, independently of the others, are as previously defined, and optionally Q and A together, or Q and A′ together, or A′, Q and A together form part of a saturated or unsaturated cyclic, bicyclic or fused ring system;
W is absent, or is selected from N(R), O or S,
wherein R is as previously defined; and
X is selected from hydrogen, X₁, where X₁ is Ra— or RbC(O)— or RbS(O)_{z}—,
where z is 1 or 2,
where Ra and Rb are independently (C₁-C₁₈)alkyl; (C₃-C₁₈)cycloalkyl; (C₃-C₁₈)cycloalkyl(C₁-C₁₈)alkyl; heterocyclic; (C₁-C₁₈)alkylheterocyclic; heterocyclic(C₆-C₂₄)aryloxy; (C₁-C₁₈)alkoxy; (C₁-C₁₈)alkoxy(C₁-C₁₈)alkyl; (C₁-C₁₂)alkyl; (C₆-C₂₄)aryloxy(C₁-C₁₈)alkyl; (C₆-C₂₄)aryloxy(C₁-C₁₈)alkoxy; (C₆-C₂₄)aryl; (C₆-C₂₄)aryl(C₁-C₁₈)alkyl; (C₆-C₂₄)aryl(C₁-C₁₈)alkylheterocyclic; (C₁-C₁₂) alkylheterocyclic; heterocyclicoxy(C₁-C₁₈)alkyl; (C₁-C₁₈)alkylamino; di(C₁-C₁₈)alkylamino; (C₆-C₂₄)arylamino; di(C₆-C₂₄)arylamino; (C₇-C₂₅)aralkylamino; or di(C₇-C₂₅)aralkylamino; any of which may be optionally substituted with one or more groups selected from —F, —Cl, —Br, —I, —CF₃, —OH, —OR_{IV}, —NH₂, —NHR_{IV}, —NR_{IV}R_{V}, —CN, —NO₂, —SH, —SR_{IV}, —SOR_{IV}, —SO₂R_{IV}, ═O, ═S, ═NOH, ═NOR_{IV}, —NHOH, —NHOR_{IV}, —CHO, where R_{IV} and R_{V} are independently (C₁-C₁₈)alkyl; (C₃-C₁₈)cycloalkyl; (C₃-C₁₈)-cycloalkyl(C₁-C₁₈)alkyl; (C₆-C₂₄)-aryl; (C₇-C₂₅)aralkyl; (C₂-C₁₈)alkenyl; (C₈-C₂₆)aralkenyl; (C₂-C₁₈)alkynyl; (C₈-C₂₆)-aralkynyl; or heterocyclic; and Re,
where Re is a group of the formula: 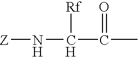
where Z has the meaning of Ra or Rb or is an acylated amino acid, azaamino acid or peptide residue, and R_{f} is the side-chain of a natural amino acid in which any functional group present is optionally protected;
Re, an optionally protected amino acid, azaamino acid or peptide residue, and, when W is N(R), then X, N and the substituent R on N together may form a saturated or unsaturated cyclic, bicyclic or fused ring system, or N, A′ and the substituent R on N together form a saturated or unsaturated cyclic, bicyclic, or fused ring.

16. A method for producing a beta-secretase complex comprising the composition of claim 15.

17. A method for inhibiting the production of beta-amyloid plaque in an animal, comprising administering to said animal an effective inhibiting amount of a compound of formula (I): 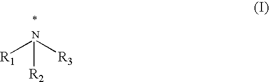
wherein R₁ is selected from R,
wherein R is selected from the group consisting of hydrogen, —R′H, —R′C(O)OR″, —R′C(O)NH₂, —R′C(O)NHR″, —R′C(O)NR″R′″, —R′NHC(O)R″, —R′NR′″C(O)R″, and —R′C(O)R″,
where R″ and R′″ are independently selected from (C₁-C₁₈)alkyl; (C₃-C₁₈)cycloalkyl; (C₃-C₁₈)cycloalkyl(Cₗ-C₁₈)alkyl; (C₆-C₂₄)aryl; (C₇-C₂₅)aralkyl; (C₂-C₁₈)alkenyl; (C₈-C₂₆)aralkenyl; (C₂-C₁₈)alkynyl; (C₈-C₂₆)aralkynyl; or heterocyclic; all optionally substituted,
where R′ is a divalent radical derived from (C₁-C₁₈)alkyl; (C₃-C₁₈)cycloalkyl; (C₃-C₁₈)cycloalkyl(C₁-C₁₈)alkyl; (C₆-C₂₄)aryl; (C₇-C₂₅)aralkyl; (C₂-C₁₈)alkenyl; (C₈-C₂₆)aralkenyl; (C₂-C₁₈)alkynyl; (C₈-C₂₆)aralkynyl; or heterocyclic; all optionally substituted;
and the moeity 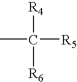
where R₄, R₅ and R₆ are independently a group R as defined above; or R₄ has the meaning of R as defined above and R₅ and R₆ taken together are ═O, ═S, ═NH or ═NR;
R₂ is 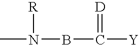
where R is as previously defined;
D is O or S;
Y is selected from hydrogen, —R or —OR, and an amino acid, aza-amino acid or peptide residue in which any functional group present is optionally protected; and
B is optionally absent or is (C₁-C₆)alkylidene, wherein any one or more —CH₂— groups may be replaced by —NR—, —NH—, —O— or —S—, provided that the compound of Formula (I) does not contain a chain of three or more atoms which are not carbon, and wherein any H atom may be substituted by a group R as previously defined;
N*, N, R₁ and R can be optionally taken together to form a cyclic diazaalkane of the formula: 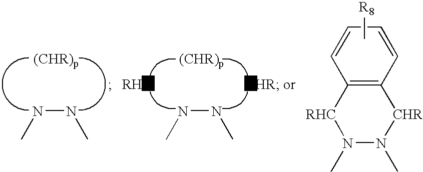
where p is 1 to 3,
each R is independently as defined above, and
R₈ is selected from R, —NH₂, —NHR, —NR₂, —COOH, —COOL, —CHO, —C(O)R, —CN, halo, —CF₃, —OL, —SR, —S(O)R, —S(O)₂R, —CONH₂, —CONHR, —CONR₂, —NHOH, —NHOL, —NO₂, ═O, ═S or —NHNH₂,
wherein each R is independently as defined above, and wherein L is independently R or a hydroxyl protecting group;
or
R₂, N* and R₄ together form a saturated or unsaturated cyclic, bicyclic or fused ring system which may be additionally substituted by —C(O)Y, where Y is as previously defined;
R₃ is X—W-A′-Q-A-, wherein: A′ and A independently are absent or (C₁-C₈)alkylidene which may be substituted with one or more substituents R as previously defined;
Q is 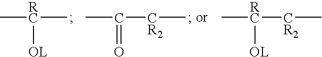
where L and each R, independently of the others, are as previously defined, and optionally Q and A together, or Q and A′ together, or A′, Q and A together form part of a saturated or unsaturated cyclic, bicyclic or fused ring system;
W is absent, or is selected from N(R), O or S,
wherein R is as previously defined; and
X is selected from hydrogen, X₁, where X, is Ra— or RbC(O)— or RbS(O)_{z}—,
where z is 1 or 2,
where Ra and Rb are independently (C₁-C₁₈)alkyl; (C₃-C₁₈)cycloalkyl; (C₃-C₁₈)cycloalkyl(C₁-C₁₈)alkyl; heterocyclic; (C₁-C₁₈)alkylheterocyclic; heterocyclic(C₆-C₂₄)aryloxy; (C₁-C₁₈)alkoxy; (C₁-C₁₈)alkoxy(C₁-C₁₈)alkyl; (C₁-C₁₂)alkyl; (C₆-C₂₄)aryloxy(C₁-C₁₈)alkyl; (C₆-C₂₄)aryloxy(C₁-C₁₈)alkoxy; (C₆-C₂₄)aryl; (C₆-C₂₄)aryl(C₁-C₁₈)alkyl; (C₆-C₂₄)aryl(C₁-C₁₈)alkylheterocyclic; (C₁-C₁₂)alkylheterocyclic; heterocyclicoxy(C₁-C₁₈)alkyl; (C₁-C₁₈)alkylamino; di(C₁-C₁₈)alkylamino; (C₆-C₂₄)arylamino; di(C₆-C₂₄)arylamino; (C₇-C₂₅)aralkylamino; or di(C₇-C₂₅)aralkylamino; any of which may be optionally substituted with one or more groups selected from —F, —Cl, —Br, —I, —CF₃, —OH, —OR_{IV}, —NH₂, —NHR_{IV}, —NR_{IV}R_{V}, —CN, —NO₂, —SH, —SR_{IV}, —SOR_{IV}, —SO₂R_{IV}, ═O, ═S, ═NOH, ═NOR_{IV}, —NHOH, —NHOR_{IV}, —CHO, where R_{IV} and R_{V} are independently (C₁-C₁₈)alkyl; (C₃-C₁₈)cycloalkyl; (C₃-C₁₈)-cycloalkyl(C₁-C₁₈)alkyl; (C₆-C₂₄)-aryl; (C₇-C₂₅)aralkyl; (C₂-C₁₈)alkenyl; (C₈-C₂₆)aralkenyl; (C₂-C₁₈)alkynyl; (C₈-C₂₆)-aralkynyll; or heterocyclic; and Re,
where Re is a group of the formula: 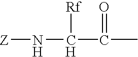
where Z has the meaning of Ra or Rb or is an acylated amino acid, azaamino acid or peptide residue, and R_{f} is the side-chain of a natural amino acid in which any functional group present is optionally protected;
Re, an optionally protected amino acid, azaamino acid or peptide residue, and, when W is N(R), then X, N and the substituent R on N together may form a saturated or unsaturated cyclic, bicyclic or fused ring system, or N, A′ and the substituent R on N together form a saturated or unsaturated cyclic, bicyclic, or fused ring.

18. The method of claim 17, wherein said animal is a human.

19. A method for treating or preventing a disease characterized by beta-amyloid deposits on or in the brain, comprising administering to a subject in need of such treatment or prevention an effective therapeutic amount of a compound of formula (I): 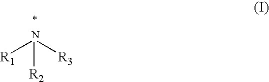
wherein R₁ is selected from R,
wherein R is selected from the group consisting of hydrogen, —R′H, —R′C(O)OR″, —R′C(O)NH₂, —R′C(O)NHR″, —R′C(O)NR″R′″, —R′NHC(O)R″, —R′NR′″C(O)R″, and —R′C(O)R″,
where R″ and R′″ are independently selected from (C₁-C₁₈)alkyl; (C₃-C₁₈)cycloalkyl; (C₃-C₁₈)cycloalkyl(C₁-C₁₈)alkyl; (C₆-C₂₄)aryl; (C₇-C₂₅)aralkyl; (C₂-C₁₈)alkenyl; (C₈-C₂₆)aralkenyl; (C₂-C₁₈)alkynyl; (C₈-C₂₆)aralkynyl; or heterocyclic; all optionally substituted,
where R′ is a divalent radical derived from (C₁-C₁₈)alkyl; (C₃-C₁₈)cycloalkyl; (C₃-C₁₈)cycloalkyl(C₁-C₁₈)alkyl; (C₆-C₂₄)aryl; (C₇-C₂₅)aralkyl; (C₂-C₁₈)alkenyl; (C₈-C₂₆)aralkenyl; (C₂-C₁₈)alkynyl; (C₈-C₂₆)aralkynyl; or heterocyclic; all optionally substituted;
and the moeity 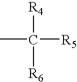
where R₄, R₅ and R₆ are independently a group R as defined above; or R₄ has the meaning of R as defined above and R₅ and R₆ taken together are ═O, ═S, ═NH or ═NR;
R₂ is 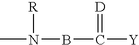
where R is as previously defined;
D is O or S;
Y is selected from hydrogen, —R or —OR, and an amino acid, aza-amino acid or peptide residue in which any functional group present is optionally protected; and
B is optionally absent or is (C₁-C₆)alkylidene, wherein any one or more —CH₂— groups may be replaced by —NR—, —NH—, —O— or —S—, provided that the compound of Formula (I) does not contain a chain of three or more atoms which are not carbon, and wherein any H atom may be substituted by a group R as previously defined;
N*, N, R₁ and R can be optionally taken together to form a cyclic diazaalkane of the formula: 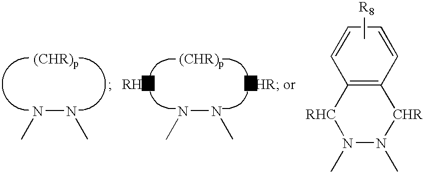
where p is 1 to 3,
each R is independently as defined above, and
R₈ is selected from R, —NH₂, —NHR, —NR₂, —COOH, —COOL, —CHO, —C(O)R, —CN, halo, —CF₃, —OL, —SR, —S(O)R, —S(O)₂R, —CONH₂, —CONHR, —CONR₂, —NHOH, —NHOL, —NO₂, ═O, ═S or —NHNH₂,
wherein each R is independently as defined above, and wherein L is independently R or a hydroxyl protecting group;
or
R₂, N* and R₄ together form a saturated or unsaturated cyclic, bicyclic or fused ring system which may be additionally substituted by —C(O)Y, where Y is as previously defined;
R₃ is X—W-A′-Q-A-, wherein: A′ and A independently are absent or (C₁-C₈)alkylidene which may be substituted with one or more substituents R as previously defined;
Q is 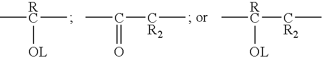
where L and each R, independently of the others, are as previously defined, and optionally Q and A together, or Q and A′ together, or A′, Q and A together form part of a saturated or unsaturated cyclic, bicyclic or fused ring system;
W is absent, or is selected from N(R), O or S,
wherein R is as previously defined; and
X is selected from hydrogen, X₁, where X₁ is Ra— or RbC(O)— or RbS(O)_{z}—,
where z is 1 or 2,
where Ra and Rb are independently (C₁-C₁₈)alkyl; (C₃-C₁₈)cycloalkyl; (C₃-C₁₈)cycloalkyl (C₁-C₁₈)alkyl; heterocyclic; (C₁-C₁₈)alkylheterocyclic; heterocyclic(C₆-C₂₄)aryloxy; (C₁-C₁₈)alkoxy; (C₁-C₁₈)alkoxy(C₁-C₁₈)alkyl; (C₁-C₁₂)alkyl; (C₆-C₂₄)aryloxy(C₁-C₁₈)alkyl; (C₆-C₂₄)aryloxy(C₁-C₁₈)alkoxy; (C₆-C₂₄)aryl; (C₆-C₂₄)aryl(C₁-C₁₈)alkyl; (C₆-C₂₄)aryl(C₁-C₁₈)alkylheterocyclic; (C₁-C₁₂)alkylheterocyclic; heterocyclicoxy(C₁-C₁₈)alkyl; (C₁-C₁₈)alkylamino; di(C₁-C₁₈)alkylamino; (C₆-C₂₄)arylamino; di(C₆-C₂₄)arylamino; (C₇-C₂₅)aralkylamino; or di(C₇-C₂₅)aralkylamino; any of which may be optionally substituted with one or more groups selected from —F, —Cl, —Br, —I, —CF₃, —OH, —OR_{IV}, —NH₂, —NHR_{IV}, —NR_{IV}R_{V}, —CN, —NO₂, —SH, —SR_{IV}, —SOR_{IV}, —SO₂R_{IV}, ═O, ═S, ═NOH, ═NOR_{IV}, —NHOH, —NHOR_{IV}, —CHO, where R_{IV} and R_{V} are independently (C₁-C₁₈)alkyl; (C₃-C₁₈)cycloalkyl; (C₃-C₁₈)-cycloalkyl(C₁-C₁₈)alkyl; (C₆-C₂₄)-aryl; (C₇-C₂₅)aralkyl; (C₂-C₁₈)alkenyl; (C₈-C₂₆)aralkenyl; (C₂-C₁₈)alkynyl; (C₈-C₂₆)-aralkynyl; or heterocyclic; and Re,
where Re is a group of the formula: 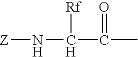
where Z has the meaning of Ra or Rb or is an acylated amino acid, azaamino acid or peptide residue, and R_{f} is the side-chain of a natural amino acid in which any functional group present is optionally protected;
Re, an optionally protected amino acid, azaamino acid or peptide residue, and, when W is N(R), then X, N and the substituent R on N together may form a saturated or unsaturated cyclic, bicyclic or fused ring system, or N, A′ and the substituent R on N together form a saturated or unsaturated cyclic, bicyclic, or fused ring.

20. A method of treatment according to claim 5, further comprising administration of one or more therapeutic agents selected from the group consisting of an antioxidant, an anti-inflammatory, a gamma secretase inhibitor, a neurotrophic agent, an acetyl cholinesterase inhibitor, a statin, P-gp inhibitors, an A beta peptide, and an anti-A beta peptide.

21. (Canceled)

22. A method of treating or preventing Alzheimer's disease in a subject in need of such treatment comprising administering a therapeutically effective amount of a compound of Formula (IA) or a pharmaceutically acceptable salt thereof: 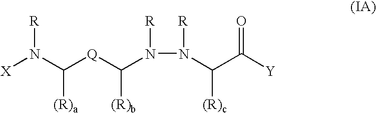
where X, Q, Y and each R is independently as previously defined,
a and b are independently 0 to 4,
c is 0 to 6,
or two R groups taken together are —(CHR₁₈)ₘ—
where m is 2-8, and
R₁₈ has the meaning of R.

23. A method of treating or preventing Alzheimer's disease in a subject in need of such treatment comprising administering a therapeutically effective amount of a compound of Formula (IB) or a pharmaceutically acceptable salt thereof: 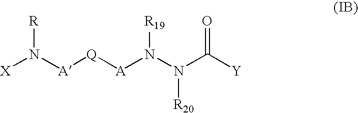
where X, R, A′, Q, A and Y are as previously defined or either or both of A and A′ are absent, and R₁₉ and R₂₀ have the meaning of R or where R₁₉, N*, N and R₂₀ together form a cyclic diazaalkane as previously defined.

24. A method of treating or preventing Alzheimer's disease in a subject in need of such treatment comprising administering a therapeutically effective amount of a compound of Formula (IC) or a pharmaceutically acceptable salt thereof: 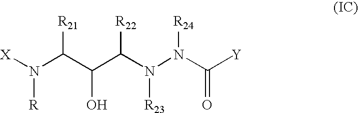
wherein R is as defined above;
R₂₁ is hydrogen, optionally substituted (C₁-C₁₂)alkyl; optionally substituted (C₆-C₁₂)aryl; optionally substituted (C₇-C₁₆)aralkyl;
R₂₂ is hydrogen, (C₁-C₈)alkyl; (C₇-C₁₆)aralkyl, or when R₂₁ and R₂₂ taken together are —(CH₂)ₙ—, wherein n is 2 to 8;
R₂₃ is hydrogen; optionally substituted (C₁-C₁₂)alkyl; (C₆-C₁₂)aryl; (C₇-C₁₆)aralkyl; or wherein R₂₂ and R₂₃ taken together are —(CHR₂₅)ₘ—, wherein m is 3-6 and R₂₅ has the meaning of R₁₀;
R₂₄ is hydrogen; optionally substituted (C₁-C₁₂)alkyl; optionally substituted (C₇-C₁₆)aralkyl; or optionally substituted (C₆-C₁₂)aryl;
or wherein NR₂₃ and NR₂₄ taken together may be a cyclic diazaalkane as previously defined; and
X and Y are as previously defined.

25. A method of treating or preventing Alzheimer's disease in a subject in need of such treatment comprising administering a therapeutically effective amount of a compound of Formula (ID) or a pharmaceutically acceptable salt thereof: 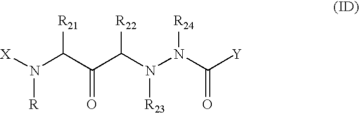
wherein R is as defined above;
R₂₁ is hydrogen, optionally substituted (C₁-C₁₂)alkyl; optionally substituted (C₆-C₁₂)aryl; optionally substituted (C₇-C₁₆)aralkyl;
R₂₂ is hydrogen, (C₁-C₈)alkyl; (C₇-C₁₆)aralkyl, or when R₂₁ and R₂₂ taken together are —(CH₂)ₙ—, wherein n is 2 to 8;
R₂₃ is hydrogen; optionally substituted (C₁-C₁₂)alkyl; (C₆-C₁₂)aryl; (C₇-C₁₆)aralkyl; or wherein R₂₂ and R₂₃ taken together are —(CHR₂₅)ₘ—, wherein m is 3-6 and R₂₅ has the meaning of R₁₀;
R₂₄ is hydrogen; optionally substituted (C₁-C₁₂)alkyl; optionally substituted (C₇-C₁₆)aralkyl; or optionally substituted (C₆-C₁₂)aryl;
or wherein NR₂₃ and NR₂₄ taken together may be a cyclic diazaalkane as previously defined; and
X and Y are as previously defined.

26. A method of treating a subject who has, or in preventing a subject from getting, a disease or condition selected from the group consisting of Alzheimer's disease, for helping prevent or delay the onset of Alzheimer's disease, for treating subjects with mild cognitive impairment (MCI) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, or diffuse Lewy body type of Alzheimer's disease and who is in need of such treatment which includes administration of a therapeutically effective amount of a compound of formula (IA) or a pharmaceutically acceptable salt thereof: 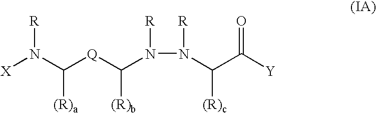
where X, Q, Y and each R is independently as previously defined,
a and b are independently 0 to 4,
c is 0 to 6,
or two R groups taken together are —(CHR₁₈)ₘ—
where m is 2-8, and
R₁₈ has the meaning of R.

27. A method of treating a subject who has, or in preventing a subject from getting, a disease or condition selected from the group consisting of Alzheimer's disease, for helping prevent or delay the onset of Alzheimer's disease, for treating subjects with mild cognitive impairment (MCI) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, or diffuse Lewy body type of Alzheimer's disease and who is in need of such treatment which includes administration of a therapeutically effective amount of a compound of formula (IB) or a pharmaceutically acceptable salt thereof: 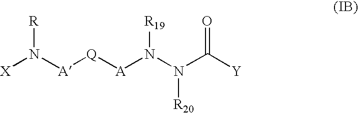
where X, R, A′, Q, A and Y are as previously defined or either or both of A and A′ are absent, and R₁₉ and R₂₀ have the meaning of R or where R₁₉, N*, N and R₂₀ together form a cyclic diazaalkane as previously defined.

28. A method of treating a subject who has, or in preventing a subject from getting, a disease or condition selected from the group consisting of Alzheimer's disease, for helping prevent or delay the onset of Alzheimer's disease, for treating subjects with mild cognitive impairment (MCI) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, or diffuse Lewy body type of Alzheimer's disease and who is in need of such treatment which includes administration of a therapeutically effective amount of a compound of formula (IC) or a pharmaceutically acceptable salt thereof: 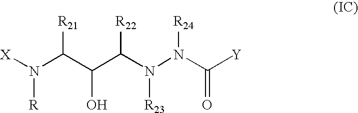
wherein R is as defined above;
R₂₁ is hydrogen, optionally substituted (C₁-C₁₂)alkyl; optionally substituted (C₆-C₁₂)aryl; optionally substituted (C₇-C₁₆)aralkyl;
R₂₂ is hydrogen, (C₁-C₈)alkyl; (C₇-Cl₆)aralkyl, or when R₂₁ and R₂₂ taken together are —(CH₂)ₙ—, wherein n is 2 to 8;
R₂₃ is hydrogen; optionally substituted (C₁-C₁₂)alkyl; (C₆-C₁₂)aryl; (C₇-C₁₆)aralkyl; or wherein R₂₂ and R₂₃ taken together are —(CHR₂₅)ₘ—, wherein m is 3-6 and R₂₅ has the meaning of R₁₀;
R₂₄ is hydrogen; optionally substituted (C₁-C₁₂)alkyl; optionally substituted (C₇-C₁₆)aralkyl; or optionally substituted (C₆-C₁₂)aryl;
or wherein NR₂₃ and NR₂₄ taken together may be a cyclic diazaalkane as previously defined; and
X and Y are as previously defined.

29. A method of treating a subject who has, or in preventing a subject from getting, a disease or condition selected from the group consisting of Alzheimer's disease, for helping prevent or delay the onset of Alzheimer's disease, for treating subjects with mild cognitive impairment (MCI) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, or diffuse Lewy body type of Alzheimer's disease and who is in need of such treatment which includes administration of a therapeutically effective amount of a compound of formula (ID) or a pharmaceutically acceptable salt thereof: 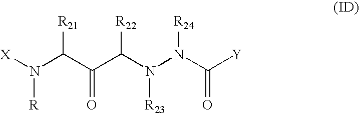
wherein R is as defined above;
R₂₁ is hydrogen, optionally substituted (C₁-C₁₂)alkyl; optionally substituted (C₆-C₁₂)aryl; optionally substituted (C₇-C₁₆)aralkyl;
R₂₂ is hydrogen, (C₁-C₈)alkyl; (C₇-C₁₆)aralkyl, or when R₂₁ and R₂₂ taken together are —(CH₂)ₙ—, wherein n is 2 to 8;
R₂₃ is hydrogen; optionally substituted (C₁-C₁₂)alkyl; (C₆-C₁₂)aryl; (C₇-C₁₆)aralkyl; or wherein R₂₂ and R₂₃ taken together are —(CHR₂₅)ₘ—, wherein m is 3-6 and R₂₅ has the meaning of R₁₀;
R₂₄ is hydrogen; optionally substituted (C₁-C₁₂)alkyl; optionally substituted (C₇-C₁₆)aralkyl; or optionally substituted (C₆-C₁₂)aryl;
or wherein NR₂₃ and NR₂₄ taken together may be a cyclic diazaalkane as previously defined; and
X and Y are as previously defined.

30. The method of claim 5, wherein the compound is of the formula B: 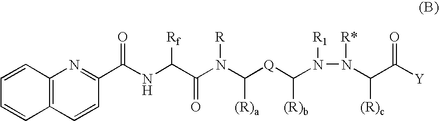
or pharmaceutically acceptable salt thereof;
wherein R_{f} is the side-chain of a natural amino acid in which any functional group present is optionally protected;
each R is independently selected from the group consisting of hydrogen, —R′H, —R′C(O)OR″, —R′C(O)NH₂, —R′C(O)NHR″, —R′C(O)NR″R′″, —R′NHC(O)R″ and —R′C(O)R″,
where R″ and R′″ are (C₁-C₁₂)alkyl, (C₃-C₁₂)cycloalkyl, (C₃-C₁₂)cycloalkyl(C₁-C₆)alkyl, (C₆-C₁₂)aryl, (C₇-C₁₆)aralkyl, (C₂-C₁₂)alkenyl, (C₈-C₁₆)aralkenyl, (C₂-C₁₂)alkynyl, (C₈-C₁₆)aralkynyl, or heterocyclic; and
R′ is an optionally substituted divalent radical derived from (C₁-C₁₂)alkyl, (C₃-C₁₂)cycloalkyl, (C₃-C₁₂)cycloalkyl(C₁-C₆)alkyl, (C₆-C₁₂)aryl, (C₇-Cl₆)aralkyl, (C₂-C₁₂)alkenyl, (C₈-C₁₆)aralkenyl, (C₂-C₁₂)alkynyl, (C₈-C₁₆)-aralkynyl, or heterocyclic; or
wherein any two R substitutents, not necessarily vicinal, taken together are optionally substituted linear (C₂-C₈)alkylidene;
R₁ and R* are independently a group R, as previously defined;
Y is hydrogen, —R or —OR, where R is as previously defined, or is an amino acid or peptide residue in which any functional group present is optionally protected;
a and b are independently 0 to 4;
c is 0 to 6; and
Q is 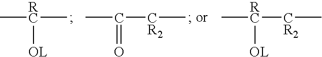
where L is R or a protecting group that protects the hydroxyl group during synthesis and/or prevents premature metabolism of the compound of formula (B), and each R, independently of the others, are as previously defined.

31. The method according to claim 30, wherein the compound is of the structure represented by formula (C) or (D): 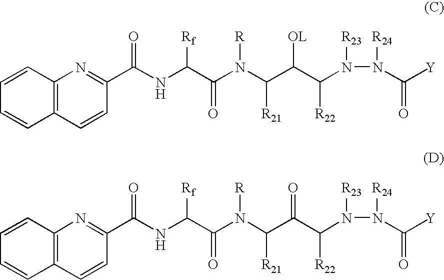
or pharmaceutically acceptable salts thereof,
wherein R is as defined in claim 30;
R₂₁ is hydrogen, optionally substituted (C₁-C₁₂)alkyl; optionally substituted (C₆-C₁₂)aryl; or optionally substituted (C₇-C₁₆)aralkyl,
R₂₂ is hydrogen, (C₁-C₈)alkyl or (C₇-C₁₆)aralkyl, or wherein R₂₁ and R₂₂ taken together are —(CH₂)ₙ—,
wherein n is 2-8;
R₂₃ is hydrogen; optionally substituted (C₁-C₁₂)alkyl; (C₆-C₁₂)aryl; (C₇-C₁₆)aralkyl; or
wherein R₂₂ and R₂₃ taken together are —(CHR₂₅)ₘ—,
wherein m is 3-6, and
R₂₅ has the meaning of R₁;
R₂₄ is hydrogen; optionally substituted (C₁-C₁₂)alkyl; optionally substituted (C₇-Cl₆)aralkyl; or optionally substituted (C₆-C₁₂)aryl;
Y and R_{f} are as defined in claim 1; and
L is R or a protecting group that protects the hydroxyl group during synthesis and/or prevents premature metabolism of the compound of formula (C).

32. The method according to claim 30, wherein the compound is selected from the group:
(i) t-butyl 3-isopropyl-3-[(2R or S,3S)-2-hydroxy-3-(N-quinaldyl-L-valyl)amino-4-phenylbutyl]carbazate;
(ii) t-butyl 3-isopropyl-3-[(2R or S,3S)-2-hydroxy-3-(N-quinaldyl-L-asparaginyl)amino-4-phenylbutyl]carbazate;
(iii) t-butyl 3-isopropyl-3-[(3S)-2-oxo-3-(N-quinaldyl-L-asparaginyl)-amino-4-phenylbutyl]carbazate;
(iv) t-butyl 3-(1-methyl-3-phenylpropyl)-3-[(2R or S,3S)-2-hydroxy-3-(N-quinaldyl-L-asparaginyl)amino-4-phenylbutyl]carbazate; and
(v) 1-[2-(2-pyridyl)methoxycarbonylamino-]benzoyl-2-[(2R or S, 3S)-2-hydroxy-3-(N-quinaldyl-L-asparaginyl)amino-4-phenylbutyl]-2-isopropyl-hydrazine;
or pharmaceutically acceptable salts thereof.

33. The method according to claim 30, wherein said compound has the formula: 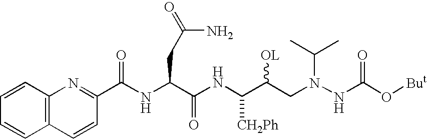
or pharmaceutically acceptable salts thereof;
where L is H or a protecting group that protects the hydroxyl group during synthesis and/or prevents premature metabolism of the compound.

34. The method according to claim 30, wherein c of the formula is 0.
